(19) United States
(12) Patent Application Publication
(10) Pub. No.: US 2009/0226400 A1
(43) Pub. Date: Sep. 10, 2009

(54) CONTINUOUS DELIVERY METHODS FOR TREATING HEPATITIS VIRUS INFECTION

(75) Inventors: Lawrence M. Blatt, San Francisco, CA (US); Brian Murphy, San Francisco, CA (US)

Correspondence Address: ROGER BRAUER, 50 HENRY AVENUE, PALISADES PARK, NJ, 07650, US

(73) Assignee: THREE RIVERS PHARMACEUTICALS, LLC, Cranberry Township, PA (US)

(21) Appl. No.: 12/420,459

(22) Filed: Apr. 8, 2009

Related U.S. Application Data
(63) Continuation of application No. 10/545,867, filed on Jun. 12, 2006, now abandoned.
(60) Provisional application No. 60/451,349, filed on Feb. 28, 2003.

(51) Int. Cl. A61P 31/12; A61K 38/21; A61M 37/00

(52) U.S. Cl. 424/85.7; 604/93.01

(57) ABSTRACT

The present invention provides methods of treating hepatitis virus infection. The methods generally involve administering an IFN-x by continuous delivery. Continuous delivery of IFN-x provides for a serum profile of IFN-x such that a sustained viral response is achieved.

### FIELD OF THE INVENTION

This invention is in the field of treatments for viral infections, in particular hepatitis virus.

### BACKGROUND OF THE INVENTION

Hepatitis C virus (HCV) infection is the most common chronic blood borne infection in the United States. Although the numbers of new infections have declined, the burden of chronic infection is substantial, with Centers for Disease Control estimates of 3.9 million (1.8%) infected persons in the United States. Chronic liver disease is the tenth leading cause of death among adults in the United States, and accounts for approximately 25,000 deaths annually, or approximately 1% of all deaths. Studies indicate that 40% of chronic liver disease is HCV-related, resulting in an estimated 8,000-10,000 deaths each year. HCV-associated end-stage liver disease is the most frequent indication for liver transplantation among adults.

Antiviral therapy of chronic hepatitis C has evolved rapidly over the last decade, with significant improvements seen in the efficacy of treatment. Nevertheless, even with combination therapy using PEGylated IFN-α plus ribavirin, 40% to 50% of patients fail therapy, i.e., are nonresponders or relapsers. These patients currently have no effective therapeutic alternative. In particular, patients who have advanced fibrosis or cirrhosis on liver biopsy are at significant risk of developing complications of advanced liver disease, including ascites, jaundice, variceal bleeding, encephalopathy, and progressive liver failure, as well as a markedly increased risk of hepatocellular carcinoma.

The high prevalence of chronic HCV infection has important public health implications for the future burden of chronic liver disease in the United States. Data derived from the National Health and Nutrition Examination Survey (NHANES III) indicate that a large increase in the rate of new HCV infections occurred from the late 1960s to the early 1980s, particularly among persons between 20 to 40 years of age. It is estimated that the number of persons with long-standing HCV infection of 20 years or longer could more than quadruple from 1990 to 2015, from 750,000 to over 3 million. The proportional increase in persons infected for 30 or 40 years would be even greater. Since the risk of HCV-related chronic liver disease is related to the duration of infection, with the risk of cirrhosis progressively increasing for persons infected for longer than 20 years, this will result in a substantial increase in cirrhosis-related morbidity and mortality among patients infected between the years of 1965-1985.

Chronic hepatitis C virus infection is characterized by intermittent or persistent elevations in serum alanine aminotransferase (ALT) levels and constant levels of HCV RNA in the circulation. Currently, approved therapies use alpha interferons derived from natural leukocytes or by recombinant methods using cDNA sequences of specific subtypes or consensus interferon-α (IFN-α). The accepted dosage regimen is a subcutaneous administration of IFN-α 2a or 2b at a dosage of about 3 million International Units (IU) tiw (three times in week) or a consensus interferon-α at a dosage of 9-15 μg tiw for a period of 24-48 weeks.

Viral kinetics during treatment regimens that include IFN-α has been examined. In general, an initial rapid decline in viral titers (early viral response; EVR) is seen in some individuals. The EVR results in an approximately 0.5- to 3-log decrease in serum HCV RNA levels in a period of 24-48 hours after initiation of treatment. An early robust response is favorable toward achieving a durable response. In some individuals, the EVR is followed by a further, less rapid decline of the virus in blood (second phase decline). The second phase decline is a slower decrease in the level of the virus over several weeks or months.

Despite the availability of approved treatment regimens discussed above, only a small fraction of the individuals treated attain a sustained viral response. Thus, there is a need in the art for improved methods for treating HCV infection. The present invention addresses this need.

### LITERATURE

U.S. Pat. Nos. 6,172,046; 6,245,740; 5,824,784; 5,372,808; 5,980,884; published international patent applications WO 96/21468; WO 96/11953; Torre et al. (2001) J. Med. Virol. 64:455-459; Bekkering et al. (2001) J. Hepatol. 34:435-440; Zeuzem et al. (2001) Gastroenterol. 120:1438-1447; Zeuzem (1999) J. Hepatol. 31:61-64; Keeffe and Hollinger (1997) Hepatol. 26:101S-107S; Wills (1990) Clin. Pharmacokinet 19:390-399; Heathcote et al. (2000) New Engl. J. Med. 343:1673-1680; Husa and Husova (2001) Bratisl. Lek Listy 102:248-252; Glue et al. (2000) Clin. Pharmacol. 68:556-567; Bailon et al. (2001) Bioconj. Chem. 12:195-202; and Neumann et al. (2001) Science 282:103; Zalipsky (1995) Adv. Drug Delivery Reviews S. 16, 157-182; Mann et al. (2001) Lancet 358:958-965; Zeuzem et al. (2000) New Engl. J. Med. 343:1666-1672; U.S. Pat. Nos. 5,985,265; 5,908,121; 6,177,074; 5,985,263; 5,711,944; 5,382,657; and 5,908,121; Osborn et al. (2002) J. Pharmacol. Exp. Therap. 303:540-548; Sheppard et al. (2003) Nat. Immunol. 4:63-68; Chang et al. (1999) Nat. Biotechnol. 17:793-797; Adolf (1995) Multiple Sclerosis 1 Suppl. 1:S44-S47.

### SUMMARY OF THE INVENTION

The present invention provides methods of treating hepatitis virus infection. The methods generally involve administering an interferon receptor agonist by continuous delivery. Continuous delivery of an interferon receptor agonist provides for a serum profile of the agonist such that a sustained viral response is achieved.

### FEATURES OF THE INVENTION

The present invention features a method of treating a hepatitis C virus (HCV) infection in a patient comprising administering a therapeutically effective amount of an interferon receptor agonist to the patient in a manner effective to achieve and maintain a sustained serum concentration of the interferon receptor agonist at a substantially steady state for a treatment period of at least about 6 weeks, or at least about 12 weeks, or at least about 24 weeks, or at least about 48 weeks, or at least about 60 weeks. Optionally, the sustained serum concentration of the interferon receptor agonist is at least about 55%, or at least about 60%, or at least about 65%, or at least about 70%, or at least about 75%, or at least about 80%, or at least about 85%, or at least about 90%, or at least about 95%, and up to about 100%, of the maximum tolerated dose (MTD) of the patient. Optionally, an implantable infusion pump is used to administer the interferon receptor agonist to the patient in a substantially continuous or continuous manner.

In some embodiments, the interferon receptor agonist is a Type I interferon receptor agonist. In other embodiments, the interferon receptor agonist is a Type II interferon receptor agonist. In other embodiments, the interferon receptor agonist is a Type III interferon receptor agonist. In some particular embodiments, IFN-γ is administered. In other particular embodiments, IFN-α is administered.

For example, the present invention features a method of treating a hepatitis C virus (HCV) infection in a patient comprising administering a therapeutically effective amount of an IFN-α to the patient in a manner effective to achieve and maintain a sustained serum concentration of the IFN-α at a substantially steady state for a treatment period of at least about 6 weeks, or at least about 12 weeks, or at least about 24 weeks, or at least about 48 weeks, or at least about 60 weeks. Optionally, the sustained serum concentration of the IFN-α is at least about 55%, or at least about 60%, or at least about 65%, or at least about 70%, or at least about 75%, or at least about 80%, or at least about 85%, or at least about 90%, or at least about 95%, and up to about 100%, of the maximum tolerated dose (MTD) of the patients Optionally, an implantable infusion pump is used to administer the IFN-α to the patient in a substantially continuous or continuous manner.

The present invention also features a method of treating a hepatitis C virus (CV) infection in a patient comprising administering to the patient a therapeutically effective amount of an interferon receptor agonist, e.g., a Type I, Type II, or Type III interferon receptor agonist, for a treatment period of at least about 6 weeks, or at least about 12 weeks, or at least about 24 weeks, or at least about 48 weeks, or at least about 60 weeks, where the area under the curve defined by serum concentration of the interferon receptor agonist as a function of time for any 8 hour period in the treatment period (AUC₈ₕᵣ) is no more than about 20% above or about 20% below, or no more than about 15% above or about 15% below, or no more than about 10% above or about 10% below, or no more than about 5% above or about 5% below, an average area under the curve defined by serum concentration of the interferon receptor agonist over time for an 8 hour interval in the treatment period (AUC_{8hr average}), and where the AUC_{8hr average} is equal to the quotient of the area under the curve defined by serum concentration of the interferon receptor agonist over time for the entirety of the treatment period (AUCₜₒₜₐₗ) divided by the number of 8 hour intervals in the treatment period (t_{total1/3days}), i.e., the AUC_{8hr average}=AUCₜₒₜₐₗ/t_{total1/3days}. Optionally, the AUC_{8hr average} is at least about 55%, or at least about 60%, or at least about 65%, or at least about 70%, or at least about 75%, or at least about 80%, or at least about 85%, or at least about 90%, or at least about 95%, and up to about 100%, of the maximum tolerated dose (MTD) of the patient. Optionally, an implantable infusion pump is used to administer the interferon receptor agonist to the patient in a substantially continuous or continuous manner. In some particular embodiments, IFN-γ is administered. In other particular embodiments, IFN-α is administered.

The present invention also features a method of treating a hepatitis C virus (HCV) infection in a patient comprising administering to the patient a therapeutically effective amount of an interferon receptor agonist, e.g., a Type I, Type II, or Type III interferon receptor agonist, for a treatment period of at least about 6 weeks, or at least about 12 weeks, or at least about 24 weeks, or at least about 48 weeks, or at least about 60 weeks, where the area under the curve defined by serum concentration of the interferon receptor agonist as a function of time for any 4 hour period in the treatment period (AUC₄ₕᵣ) is no more than about 20% above or about 20% below, or no more than about 15% above or about 15% below, or no more than about 10% above or about 10% below, or no more than about 5% above or about 5% below, an average area under the curve defined by serum concentration of the interferon receptor agonist over time for a 4 hour interval in the treatment period (AUC_{4hr average}), and where the AUC_{4hr average} is equal to the quotient of the area under the curve defined by serum concentration of the interferon receptor agonist over time for the entirety of the treatment-period (AUCₜₒₜₐₗ) divided by the number of 4 hour intervals in the treatment period (t_{total1/6days}), i.e., the AUC_{4hr average}=AUCₜₒₜₐₜ/t_{total1/6days}. Optionally, the AUC_{4hr average} is at least about 55%, or at least about 60%, or at least about 65%, or at least about 70%, or at least about 75%, or at least about 80%, or at least about 85%, or at least about 90%, or at least about 95%, and up to about 100%, of the maximum tolerated dose (MTD) of the patient. Optionally, an implantable infusion pump is used to administer the interferon receptor agonist to the patient in a substantially continuous or continuous manner. In some particular embodiments, IFN-γ is administered. In other particular embodiments, IFN-α is administered.

The present invention also features a method of treating a hepatitis C virus (HCV) infection in a patient comprising administering to the patient a therapeutically effective amount of an interferon receptor agonist, e.g., a Type I, Type II, or Type III interferon receptor agonist, for a treatment period of at least about 6 weeks, or at least about 12 weeks, or at least about 24 weeks, or at least about 48 weeks, or at least about 60 weeks, where the area under the curve defined by serum concentration of the interferon receptor agonist as a function of time for any 3 hour period in the treatment period (AUC₃ₕᵣ) is no more than about 20% above or about 20% below, or no more than about 15% above or about 15% below, or no more than about 10% above or about 10% below, or no more than about 5% above or about 5% below, an average area under the curve defined by serum concentration of the interferon receptor agonist over time for a 3 hour interval in the treatment period (AUC_{3hr average}), and where the AUC_{3hr average} is equal to the quotient of the area under the curve defined by serum concentration of the interferon receptor agonist over time for the entirety of the treatment period (AUCₜₒₜₐₗ) divided by the number of 3 hour intervals in the treatment period (t_{total1/8days}), i.e., the AUC_{3hr average}=AUCₜₒₜₐₗ/t_{total1/8days}. Optionally, the AUC_{3hr average} is at least about 55%, or at least about 60%, or at least about 65%, or at least about 70%, or at least about 75%, or at least about 80%, or at least about 85%, or at least about 90%, or at least about 95%, and up to about 100%, of the maximum tolerated dose (MTD) of the patient. Optionally, an implantable infusion pump is used to administer the interferon receptor agonist to the patient in a substantially continuous or continuous manner. In some particular embodiments, IFN-γ is administered. In other particular embodiments, IFN-α is administered.

The present invention also features a method of treating a hepatitis C virus (HCV) infection in a patient comprising administering to the patient a therapeutically effective amount of an interferon receptor agonist, e.g., a Type I, Type II, or Type III interferon receptor agonist, for a treatment period of at least about 6 weeks, or at least about 12 weeks, or at least about 24 weeks, or at least about 48 weeks, or at least about 60 weeks, where the area under the curve defined by serum concentration of the interferon receptor agonist as a function of time for any 2 hour period in the treatment period (AUC₂ₕᵣ) is no more than about 20% above or about 20% below, or no more than about 15% above or about 15% below, or no more than about 10% above or about 10% below, or no more than about 5% above or about 5% below, an average area under the curve defined by serum concentration of the interferon receptor agonist over time for a 2 hour interval in the treatment period (AUC_{2hr average}), and where the AUC_{2hr average} is equal to the quotient of the area under the curve defined by serum concentration of the interferon receptor agonist over time for the entirety of the treatment period (AUCₜₒₜₐₗ) divided by the number of 2 hour intervals in the treatment period (t_{total1/12days}), i.e., the AUC_{2hr average}=AUCₜₒₜₐₗ/t_{total1/12days}. Optionally, the AUC_{2hr average} is at least about 55%, or at least about 60%, or at least about 65%, or at least about 70%, or at least about 75%, or at least about 80%, or at least about 85%, or at least about 90%, or at least about 95%, and up to about 100%, of the maximum tolerated dose (MTD) of the patient. Optionally, an implantable infusion pump is used to administer the interferon receptor agonist to the patient in a substantially continuous or continuous manner. In some particular embodiments, IFN-γ is administered. In other particular embodiments, IFN-α is administered.

The present invention also features a method treating a hepatitis C virus (HCV) infection in a patient comprising administering a therapeutically effective amount of an interferon receptor agonist, e.g., a Type I, Type II, or Type III interferon receptor agonist, for a treatment period of at least about 6 weeks, or at least about 12 weeks, or at least about 24 weeks, or at least about 48 weeks, or at least about 60 weeks, where the area under the curve defined by serum concentration of the interferon receptor agonist as a function of time for any 1 hour period in the treatment period (AUC₁ₕᵣ) is no more than about 20% above or about 20% below, or no more than about 15% above or about 15% below, or no more than about 10% above or about 10% below, or no more than about 5% above or about 5% below, an average area under the curve defined by serum concentration over time for a 1 hour interval in the treatment period (AUC_{1hr average}), and where the AUC_{1hr average} is equal to the quotient of the area under the curve defined by serum concentration of the interferon receptor agonist as a function of time for the entirety of the treatment period (AUCₜₒₜₐₗ) divided by the number of hours in the treatment period (tₜₒₜₐₗₕᵣₛ), i.e., the AUC_{1hr average}=AUCₜₒₜₐₗ/tₜₒₜₐₗₕᵣₛ. Optionally, the AUC_{1hr average} is at least about 55%, or at least about 60%, or at least about 65%, or at least about 70%, or at least about 75%, or at least about 80%, or at least about 85%, or at least about 90%, or at least about 95%, and up to about 100%, of the maximum tolerated dose (NM) of the patient. Optionally, an implantable infusion pump is used to administer the interferon receptor agonist to the patient in a substantially continuous or continuous manner. In some particular embodiments, IFN-γ is administered. In other particular embodiments, IFN-α is administered.

The present invention also features a method of treating a hepatitis C virus (HCV) infection in a patient comprising administering a therapeutically effective amount of an interferon receptor agonist, e.g., a Type I, Type II, or Type III interferon receptor agonist, to the patient in a manner effective to achieve an initial dosage phase followed by a sustained dosage phase consisting of at least one sustained dosage interval, where during the initial dosage phase an initial serum concentration of the interferon receptor agonist is achieved within a period of time of about 12 hours to about 48 hours, where during the first sustained dosage interval a first sustained serum concentration of the interferon receptor agonist is achieved and maintained at a substantially steady state for a period of time of at least about 5 days, where the first sustained serum concentration is at least about 80% and up to about 200% of the initial serum concentration, and during any following sustained dosage interval a following sustained serum concentration of the interferon receptor agonist is achieved and maintained at a substantially steady state for a period of time of at least about 5 days, where the following sustained serum concentration is at least about 20% of the first sustained serum concentration of the interferon receptor agonist and at least about 50% and up to about 200% of the sustained serum concentration of the interferon receptor agonist in the preceding sustained dosage interval, and where the duration of the interferon receptor agonist therapy is at least about 6 weeks, or at least about 12 weeks, or at least about 24 weeks, or at least about 48 weeks, or at least about 60 weeks. In some particular embodiments, IFN-γ is administered. In other particular embodiments, IFN-α is administered.

In one embodiment, the method of the invention provides for a sustained serum concentration in every following sustained dosage interval that is at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 100%, of the sustained serum concentration of the interferon receptor agonist in the first sustained dosage interval.

In another embodiment, the method of the invention provides for administering an interferon receptor agonist, e.g., a Type I, Type II, or Type III interferon receptor agonist, to the patient in a substantially continuous or continuous manner during at least the sustained dosage phase. Optionally, the sustained dosage phase consists of a single sustained dosage interval. In some particular embodiments, IFN-γ is administered. In other particular embodiments, IFN-α is administered.

In another embodiment, the method of the invention provides for administering an interferon receptor agonist, e.g., a Type I, Type II, or Type III interferon receptor agonist, to the patient in a substantially continuous or continuous manner by an implantable infusion pump during at least the sustained dosage phase. Optionally, the implantable infusion pump can be used to (i) administer to the patient a single bolus dose of the interferon receptor agonist to achieve the initial serum concentration during the initial dosage phase and (ii) administer to the patient a pre-selected amount of the interferon receptor agonist per day by substantially continuous or continuous infusion to achieve and maintain the sustained serum concentration for each sustained dosage interval. Optionally, the interferon receptor agonist is IFN-α, the implantable infusion pump is installed for subcutaneous delivery of the IFN-α, the bolus dose is at least about 3 million Units (MU) of the IFN-α, and/or the pre-selected amount of the IFN-α is at least about 3 MU per day. Optionally, the sustained dosage phase consists of a single sustained dosage interval. In some particular embodiments, IFN-γ is administered. In other particular embodiments, IFN-α is administered.

In another embodiment, the method of the invention provides for administering to the patient a single bolus dose of the interferon receptor agonist, e.g., a Type I, Type II, or Type III interferon receptor agonist, by subcutaneous injection to achieve the initial serum concentration of the interferon receptor agonist during the initial dosage phase. Optionally, the interferon receptor agonist is administered to the patient in substantially continuous or continuous manner by an implantable infusion pump that delivers a pre-selected amount of the interferon receptor agonist per day to achieve and maintain the sustained serum concentration of the interferon receptor agonist for each sustained dosage interval. Optionally, the interferon receptor agonist is an IFN-α and the pre-selected amount is at least about 9 MU of the IFN-α per day and is administered to the patient by subcutaneous infusion. Optionally, the sustained dosage phase consists of a single sustained dosage interval. In some particular embodiments, IFN-γ is administered. In other particular embodiments, IFN-α is administered.

In another embodiment, the method of the invention provides for administering an interferon receptor agonist, e.g., a Type I, Type II, or Type III interferon receptor agonist, to the patient in a substantially continuous or continuous manner during the initial and sustained dosage phases. Optionally, the interferon receptor agonist is administered to the patient in a substantially continuous or continuous manner by an implantable infusion pump during the initial and sustained dosage phases. Optionally, the implantable infusion pump is controlled to deliver a pre-selected amount of the interferon receptor agonist per day to achieve the initial serum concentration of the interferon receptor agonist during the initial dosage phase and to achieve and maintain the sustained serum concentration of the interferon receptor agonist for each sustained dosage interval. Optionally, the interferon receptor agonist is IFN-α and the pre-selected amount is at least about 9 MU of the IFN-α per day and is administered to the patient by subcutaneous infusion. Optionally, the sustained dosage phase consists of a single sustained dosage interval. In some particular embodiments, IFN-γ is administered. In other particular embodiments, IFN-α is administered.

In another embodiment, the invention provides any of the above-described methods in which the sustained serum concentration of the interferon receptor agonist in first sustained dosage interval is at least about 85%, or at least about 90%, or at least about 95%, of the initial serum concentration of the interferon receptor agonist. Optionally, the sustained dosage phase consists of a single sustained dosage interval.

In another embodiment, the invention provides any of the above-described methods in which the initial serum concentration of the interferon receptor agonist and the sustained concentration of the interferon receptor agonist in each sustained dosage interval are substantially the same. Optionally, the sustained dosage phase consists of a single sustained dosage interval.

In another embodiment, the invention provides any of the above-described methods in which the initial serum concentration of the interferon receptor agonist is at least about 55%, or at least about 60%, or at least about 65%, or at least about 70%, or at least about 75%, or at least about 80%, or at least about 85%, or at least about 90%, or at least about 95%, and up to about 100%, of the maximum tolerated dose (MID) of the patient.

In another embodiment, the invention provides any of the above-described methods in which there is more than one sustained dosage interval and the sustained serum concentration of the interferon receptor agonist in the last sustained dosage interval is at least about 55%, or at least about 60%, or at least about 65%, or at least about 70%, or at least about 75%, or at least about 80%, or at least about 85%, or at least about 90%, or at least about 95%, and up to about 100%, of the maximum tolerated dose (MTD) of the patient.

In another embodiment, the invention provides any of the above-described methods in which the sustained serum concentration of the interferon receptor agonist in the first sustained dosage interval is at least about 100%, and up to about 150%, of the initial serum concentration of the interferon receptor agonist, and the sustained serum concentration of the interferon receptor agonist in any following sustained dosage interval is at least about 90%, or at least about 100%, and up to about 150%, of the sustained serum concentration of the interferon receptor agonist in the preceding sustained dosage interval.

In another embodiment, the invention provides any of the above-described methods in which the sustained dosage phase consists of only two sustained dosage intervals (the first sustained dosage interval and a single following sustained dosage interval), the first sustained serum concentration is about 100% of the initial serum concentration, the initial dosage phase and the first sustained dosage interval extend for a combined period of time of about 4 weeks, and the sustained serum concentration of the interferon receptor agonist in the following sustained dosage interval is about 150% of the sustained serum concentration of the interferon receptor agonist in the first sustained dosage interval.

In another embodiment, the invention provides any of the above-described methods in which the interferon receptor agonist is administered to the patient in a substantially continuous or continuous manner during the initial and sustained dosage phases.

In another embodiment, the invention provides any of the above-described methods in which the sustained dosage phase consists of two or three sustained dosage intervals, where the sustained serum concentration of the interferon receptor agonist in each following sustained dosage interval is at least about 50% and up to about 70% of the sustained serum concentration of the interferon receptor agonist in the preceding sustained dosage interval.

In another embodiment, the invention provides any of the above-described methods in which the sustained dosage phase consists of two sustained dosage intervals, and the sustained serum concentration of the interferon receptor agonist in the following sustained dosage interval is at least about 50% and up to about 70% of the sustained serum concentration of the interferon receptor agonist in the first sustained dosage interval. Optionally, the first sustained dosage interval and the initial dosage phase extend for a combined period of time of about 4 weeks.

In another embodiment, the invention provides any of the above-described methods in which the sustained dosage phase consists of three sustained dosage intervals, the sustained serum concentration of the interferon receptor agonist in the second sustained dosage interval (the first to occur of the following sustained dosage intervals) is at least about 60% and up to about 70% of the first sustained serum concentration of the interferon receptor agonist, the sustained serum concentration of the interferon receptor agonist in the third sustained dosage interval (the last to occur of the following sustained dosage intervals) is about 50% of the sustained serum concentration of the interferon receptor agonist in the second sustained dosage interval, the initial dosage phase and the first sustained dosage interval extend for a combined period of time of about 4 weeks, and the second sustained dosage interval extends for a period of time of about 8 weeks.

In another embodiment, the invention provides any of the above-described methods in which the initial serum concentration of the interferon receptor agonist is achieved within a period of time of about 24 hours.

In another embodiment, the invention provides any of the above-described methods in which for every sustained dosage interval the area under the curve defined by serum concentration of the interferon receptor agonist as a function of time for any 8 hour period in the sustained dosage interval (AUC₈ₕᵣ) is no more than about 20% above or about 20% below, or no more than about 15% above or about 15% below, or no more than about 10% above or about 10% below, or no more than about 5% above or about 5% below, an average area under the curve defined by serum concentration of the interferon receptor agonist as a function of time for an 8 hour period in the sustained dosage interval (AUC_{8hr average}), where the AUC_{8hr average} is equal to the quotient of the area under the curve defined by serum concentration of the interferon receptor agonist as a function of time for the entirety of the sustained dosage interval (AUCₜₒₜₐₗ) divided by the total number of 8 hour periods in the sustained dosage interval (t_{total1/3days}) i.e., the AUC_{8hr average}=AUCₜₒₜₐₗ/t_{total1/3days}. In some particular embodiments, the interferon receptor agonist is IFN-γ. In other particular embodiments, the interferon receptor agonist is IFN-α.

In another embodiment, the invention provides any of the above-described methods in which for every sustained dosage interval the area under the curve defined by serum concentration of the interferon receptor agonist as a function of time for any 4 hour period in the sustained dosage interval (AUC₄ₕᵣ) is no more than about 20% above or about 20% below, or no more than about 15% above or about 15% below, or no more Man about 10% above or about 10% below, or no more than about 5% above or about 5% below, an average area under the curve defined by serum concentration of the interferon receptor agonist as a function of time for a 4 hour period in the sustained dosage interval (AUC_{4hr average}), where the AUC₄ₕᵣ average is equal to the quotient of the area under the curve defined by serum concentration of the interferon receptor agonist as a function of time for the entirety of the sustained dosage interval (AUCₜₒₜₐₗ) divided by the total number of 4 hour periods in the sustained dosage interval (t_{total1/6days}), i.e., the AUC_{4hr average}=AUCₜₒₜₐₗ₁/t_{total1/6days}.

In another embodiment, the invention provides any of the above-described methods in which for every sustained dosage interval the area under the curve defined by serum concentration of the interferon receptor agonist as a function of time for any 3 hour period in the sustained dosage interval (AUC₃ₕᵣ) is no more than about 20% above or about 20% below, or no more than about 15% above or about 15% below, or no more than about 10% above or about 10% below, or no more than about 5% above or about 5% below, an average area under the curve defined by serum concentration of the interferon receptor agonist as a function of time for a 3 hour period in the sustained dosage interval (AUC_{3hr average}), where the AUC_{3hr average} is equal to the quotient of the area under the curve defined by serum concentration of the interferon receptor agonist as a function of time for the entirety of the sustained dosage interval (AUCₜₒₜₐₗ) divided by the total number of 3 hour periods in the sustained dosage interval (t_{total1/8days}), i.e., the AUC_{3hr average}=AUC_{total1/8days}.

In another embodiment, the invention provides any of the above-described methods in which for every sustained dosage interval the area under the curve defined by serum concentration of the interferon receptor agonist as a function of time for any 2 hour period in the sustained dosage interval (AUC₂ₕᵣ) is no more than about 20% above or about 20% below, or no more than about 15% above or about 15% below, or no more than about 10% above or about 10% below, or no more than about 5% above or about 5% below, an average area under the curve defined by serum concentration of the interferon receptor agonist as a function of time for a 2 hour period in the sustained dosage interval (AUC_{2hr average}), where the AUC_{2hr average} is equal to the quotient of the area under the curve defined by serum concentration of the interferon receptor agonist as a function of time for the entirety of the sustained dosage interval (AUCₜₒₜₐₗ) divided by the total number of 2 hour periods in the sustained dosage interval (t_{total1/12days}), i.e., the AUC_{2hr average}=AUCₜₒₜₐₗ/t_{total1/12days}.

In another embodiment, the invention provides any of the above-described methods in which for every sustained dosage interval the area under the curve defined by serum concentration of the interferon receptor agonist as a function of time for any 1 hour period in the sustained dosage interval (AUC₂ₕᵣ) is no more than about 20% above or about 20% below, or no more than about 15% above or about 15% below, or no more than about 10% above or about 10% below, or no more than about 5% above or about 5% below, an average area under the curve defined by serum concentration of the interferon receptor agonist as a function of time for a 2 hour period in the sustained dosage interval (AUC_{1h average}), where the AUC_{2hr average} is equal to the quotient of the area under the curve defined by serum concentration of the interferon receptor agonist as a function of time for the entirety of the sustained dosage interval (AUCₜₒₜₐₗ) divided by the total number of hours in the sustained dosage interval (tₜₒₜₐₗₕᵣₛ), i.e., the AUC_{1hr average}=AUCₜₒₜₐₗ/tₜₒₜₐₗₕᵣₛ.

In some embodiments, the invention provides any one of the above-described methods in which the interferon receptor agonist is a Type I interferon receptor agonist. In other embodiments, the invention provides any one of the above-described methods in which the interferon receptor agonist is a Type II interferon receptor agonist. In other embodiments, the invention provides any one of the above-described methods in which the interferon receptor agonist is a Type III interferon receptor agonist.

In another embodiment, the invention provides any of the above-described methods in which the interferon receptor agonist is a consensus interferon. Optionally, the consensus interferon is INFERGEN® interferon alfacon-1. In another embodiment, the invention provides any of the above-described methods in which the interferon receptor agonist is IFN-α 2a or IFN-α 2b.

In other embodiments, the invention provides any one of the above-described methods in which the interferon receptor agonist is an IFN-β. In other embodiments, the invention provides any one of the above-described methods in which the interferon receptor agonist is IFN-tau. In other embodiments, the invention provides any one of the above-described methods in which the interferon receptor agonist is IFN-M.

In other embodiments, the invention provides any one of the above-described methods in which the interferon receptor agonist is an IFN-γ.

In another embodiment, the invention provides any of the above-described methods in which a therapeutically effective amount of ribavirin is also administered to the patient for the duration of the interferon receptor agonist therapy. Optionally, the method provides administering to the patient about 800 mg to about 1200 mg ribavirin orally per day for the duration of the interferon receptor agonist therapy. Optionally, the method provides for administering to the patient (a) 1000 mg ribavirin orally per day if the patient has a body weight less than 75 kg or (b) 1200 mg ribavirin orally per day of the patient has a body weight greater than or equal to 75 kg, where the daily dosage of ribavirin is administered to the individual in 2 divided doses per day for the duration of the interferon receptor agonist therapy.

The present invention also features a method of treating hepatitis C virus (HCV) infection in a patient comprising administering a therapeutically effective amount of an interferon receptor agonist to the patient by substantially continuous or continuous delivery of a pre-selected amount of the interferon receptor agonist each day for a treatment period of at least about 12 weeks, or at least about 24 weeks, or at least about 48 weeks, or at least about 60 weeks. Optionally, the pre-selected amount of the interferon receptor agonist per day is at least about 55%, or at least about 60%, or at least about 65%, or at least about 70%, or at least about 75%, or at least about 80%, or at least about 85%, or at least about 90%, or at least about 95%, and up to about 100%, of the maximum tolerated dose (MTD) of the patient. Optionally, an implantable infusion pump is used to administer the interferon receptor agonist to the patient in a substantially continuous or continuous manner.

The invention also features a method for treating a hepatitis C virus (HCV) infection in a patient, comprising administering to the patient a therapeutically effective amount of an interferon receptor agonist for a treatment period of at least about 6 weeks, or at least about 12 weeks, or at least about 24 weeks, or at least about 48 weeks, or at least about 60 weeks, where each day of the treatment period the patient receives by substantially continuous or continuous delivery an amount of the interferon receptor agonist that is no more than about 20% above or about 20% below an average daily dosage of the interferon receptor agonist (ADD_{IFNRa}), and where the ADD_{INFRa} is equal to the aggregate amount of the interferon receptor agonist administered to the patient in the treatment period divided by the number of days in the treatment period. Optionally, each day of the treatment period the patient receives an amount of the interferon receptor agonist that is no more than about 15% above or about 15% below, or no more than about 10% above or about 10% below, or no more than about 5% above or about 5% below, or is substantially the same as, the ADD_{IFNRa}.

In another embodiment, the method of the invention provides any of the above-described methods in which the area under the curve of serum concentration of the interferon receptor agonist over time for any 8 hour interval in the treatment period (AUC₈ₕᵣ) is no more than about 20% above or about 20% below the average area under the curve of serum concentration of the interferon receptor agonist over time for an 8 hour interval during the treatment period (AUC_{8hr average}), where the (AUC_{8hr average}) is equal to the quotient of the area under the curve of serum concentration of the interferon receptor agonist over the entirety of the treatment period (AUCₜₒₜₐₗ) divided by the number of 8 hour intervals in the treatment period (t_{total1/3days}). Optionally, each AUC₈ₕᵣ is no more than about 15% above or about 15% below, or no more than about 10% above or about 10% below, or no more than about 5% above or about 5% below, the AUC_{8hr average}.

In another embodiment, the invention provides any of the above-described methods in which the interferon receptor agonist is IFN-α, and the pre-selected amount of the IFN-α or the ADD_{IFNRa} is at least about 0.5 million Units (MU), or at least about 1.0 MU, or at least about 1.5 MU, or at least about 2.0 MU, or at least about 2.5 MU, or at least about 3 MU, or at least about 6 MU, or at least about 9 MU, or at least about 12 MU, or at least about 15 MU, or at least about 18 MU, or at least about 21 MN, or at least about 24 MU, or at least about 27 MN, or at least about 30 MU, administered subcutaneously.

In another embodiment, the invention provides any of the above-described methods in which the interferon receptor agonist is IFN-α, and the pre-selected amount of the IFN-α or the ADD_{IFNRa} is at least about 0.5 μg, or at least about 1.5 μg, or at least about 2.0 μg, or at least about 2.5 μg, or at least about 3 μg, or at least about 6 μg, or at least about 9 μg, or at least about 12 μg, or at least about 15 μg, or at least about 18 μg, or at least about 21 μg, or at least about 24 μg, or at least about 27 μg, or at least about 30 μg, of a consensus interferon administered subcutaneously.

The invention also features a method of treating a hepatitis C virus (HCV) infection in an patient by administering a therapeutically effective amount of an interferon receptor agonist to the patient in an initial dosage phase followed by a sustained dosage phase consisting of at least one sustained dosage interval, where the initial dosage phase extends for a period of time of about 12 hours to about 48 hours during which an initial pre-selected amount of the interferon receptor agonist is administered to the individual by a selected route of administration, where during the first sustained dosage interval a first sustained pre-selected amount of the interferon receptor agonist is administered to the individual each day by the selected route of administration in a substantially continuous or continuous manner, where the first sustained pre-selected amount of the interferon receptor agonist is at least about 80% and up to about 200% of the initial pre-selected amount of the interferon receptor agonist, and during any following sustained dosage interval a following sustained pre-selected amount of the interferon receptor agonist is administered to the individual each day by the selected route of administration in a substantially continuous or continuous manner, where the following sustained pre-selected amount of the interferon receptor agonist is at least about 20% of the first sustained pre-selected amount of the interferon receptor agonist and at least about 50% and up to about 200% of the sustained pre-selected amount of the interferon receptor agonist in the preceding sustained dosage interval, where each sustained dosage interval extends for a period of time of at least about 5 days, and where the duration of the interferon receptor agonist therapy extends for a period of time of at least about 6 weeks, or at least about 12 weeks, or at least about 24 weeks, or at least about 48 weeks, or at least about 60 weeks.

In another embodiment, the method of the invention provides that for every sustained dosage interval the area under the curve defined by serum concentration of the interferon receptor agonist as a function of time for any 8 hour period in the sustained dosage interval (AUC₈ₕᵣ) is no more than about 20% above or about 200% below, or no more than about 15% above or about 15% below, or no more than about 10% above or about 10% below, or no more than about 5% above or about 5% below, an average area under the curve defined by serum concentration of the interferon receptor agonist as a function of time for an 8 hour period in the sustained dosage interval (AUC_{8hr average}), where the AUC_{8hr average} is equal to the quotient of the area under the curve defined by serum concentration of the interferon receptor agonist as a function of time for the entirety of the sustained dosage interval (AUCₜₒₜₐₗ) divided by the total number of 8 hour periods in the sustained dosage interval (t_{total1/3days}), i.e., the AUC_{8hr average}=AUCₜₒₜₐₗ/t_{total1/3days}.

In another embodiment, the method of the invention provides that for every sustained dosage interval the area under the curve defined by serum concentration of the interferon receptor agonist as a function of time for any 4 hour period in the sustained dosage interval (AUC₄ₕᵣ) is no more than about 20% above or about 20% below, or no more than about 15% above or about 15% below, or no more than about 10% above or about 10% below, or no more than about 5% above or about 5% below, an average area under the curve defined by serum concentration of the interferon receptor agonist as a function of time for a 4 hour period in the sustained dosage interval (AUC_{4hr average}), where the AUC_{4hr average} is equal to the quotient of the area under the curve defined by serum concentration of the interferon receptor agonist as a function of time for the entirety of the sustained dosage interval (AUCₜₒₜₐₗ) divided by the total number of 4 hour periods in the sustained dosage interval (t_{total1/6days}), i.e., the AUC_{4hr average}=AUCₜₒₜₐₗ/t_{total1/6days}.

In another embodiment, the method of the invention provides that for every sustained dosage interval the area under the curve defined by serum concentration of the interferon receptor agonist as a function of time for any 3 hour period in the sustained dosage interval (AUC₃ₕᵣ) is no more than about 20% above or about 20% below, or no more than about 15% above or about 15% below, or no more than about 10% above or about 10% below, or no more than about 5% above or about 5% below, an average area under the curve defined by serum concentration of the interferon receptor agonist as a function of time for a 3 hour period in the sustained dosage interval (AUC_{3hr average}), where the AUC_{3hr average} is equal to the quotient of the area under the curve defined by serum concentration of the interferon receptor agonist as a function of time for the entirety of the sustained dosage interval (AUCₜₒₜₐₗ) divided by the total number of 3 hour periods in the sustained dosage interval (t_{total1/8days}), i.e., the AUC_{3hr average}=AUCₜₒₜₐₗ/t_{total1/8days}.

In another embodiment, the method of the invention provides that for every sustained dosage interval the area under the curve defined by serum concentration of the interferon receptor agonist as a function of time for any 2 hour period in the sustained dosage interval (AUC₂ₕᵣ) is no more than about 20% above or about 20% below, or no more than about 15% above or about 15% below, or no more than about 10% above or about 10% below, or no more than about 5% above or about 5% below, an average area under the curve defined by serum concentration of the interferon receptor agonist as a function of time for a 2 hour period in the sustained dosage interval (AUC_{2hr average}), where the AUC_{2hr average} is equal to the quotient of the area under the curve defined by serum concentration of the interferon receptor agonist as a function of time for the entirety of the sustained dosage interval (AUCₜₒₜₐₗ) divided by the total number of 2 hour periods in the sustained dosage interval (t_{total1/12days}), i.e., the AUC_{2hr average}=AUCₜₒₜₐₗ/t_{total1/12days}.

In another embodiment, the method of the invention provides that for every sustained dosage interval the area under the curve defined by serum concentration of the interferon receptor agonist as a function of time for any 1 hour period in the sustained dosage interval (sustained AUC₁ₕᵣ) is no more than about 20% above or about 20% below, or no more than about 15% above or about 15% below, or no more than about 10% above or about 10% below, or no more than about 5% above or about 5% below, an average area under the curve defined by serum concentration of the interferon receptor agonist as a function of time for a 1 hour period in the sustained dosage interval (AUC_{1hr average}), where the AUC_{1hr average} is equal to the quotient of the area under the curve defined by serum concentration of the interferon receptor agonist as a function of time for the entirety of the sustained dosage interval (AUCₜₒₜₐₗ) divided by the total number of hours in the sustained dosage interval (tₜₒₜₐₗₕᵣₛ) i.e., the AUC_{1hr average}=AUCₜₒₜₐₗ/tₜₒₜₐₗₕᵣₛ.

In another embodiment, the method of the invention provides for administering the interferon receptor agonist to the patient in a substantially continuous or continuous manner during the initial dosage phase and the sustained dosage phase. Optionally, an implantable infusion pump is used to administer the interferon receptor agonist to the patient in a substantially continuous or continuous manner during the initial dosage phase and the sustained dosage phase. Optionally, the sustained dosage phase consists of a single sustained dosage interval.

In another embodiment, the method of the invention provides for administering the interferon receptor agonist to the patient in a substantially continuous or continuous manner by an implantable infusion pump during the sustained dosage phase. Optionally, the pump is implanted and used to administer the initial pre-selected amount of the interferon receptor agonist as a bolus at the beginning of the initial dosage phase. Optionally, the sustained dosage phase consists of a single sustained dosage interval.

In another embodiment, the method of the invention provides for administering the initial pre-selected amount of the interferon receptor agonist to the patient by bolus injection at the beginning of the initial dosage phase. Optionally, an implantable infusion pump is used to administer the interferon receptor agonist to the patient in a substantially continuous or continuous manner during the sustained dosage phase. Optionally, the sustained dosage phase consists of a single sustained dosage interval.

In another embodiment, the invention provides any of the above-described methods in which the interferon receptor agonist is administered to the patient subcutaneously during the initial and sustained dosage phases. Optionally, the sustained dosage phase consists of a single sustained dosage interval.

In some embodiments, the invention provides any one of the above-described methods in which the interferon receptor agonist is a Type I interferon receptor agonist. In other embodiments, the invention provides any one of the above-described methods in which the interferon receptor agonist is a Type II interferon receptor agonist. In other embodiments, the invention provides any one of the above-described methods in which the interferon receptor agonist is a Type III interferon receptor agonist.

In another embodiment, the invention provides any of the above-described methods in which the interferon receptor agonist is a consensus interferon. Optionally, the consensus interferon is INFERGEN® interferon alfacon-1.

In another embodiment, the invention provides any of the above-described methods in which the interferon receptor agonist is IFN-α 2a or 2b.

In other embodiments, the invention provides any one of the above-described methods in which the interferon receptor agonist is an IFN-β. In other embodiments, the invention provides any one of the above-described methods in which the interferon receptor agonist is IFN-tau. In other embodiments, the invention provides any one of the above-described methods in which the interferon receptor agonist is IFN-ω.

In other embodiments, the invention provides any one of the above-described methods in which the interferon receptor agonist is an IFN-γ.

In another embodiment, the invention provides any of the above-described methods in which the sustained pre-selected amount of the interferon receptor agonist of the last sustained dosage interval is at least about 55%, or at least about 60%, or at least about 65%, or at least about 70%, or at least about 75%, or at least about 80%, or at least about 85%, or at least about 90%, or at least about 95%, and up to about 100%, of the maximum tolerated dose (MTD) of the patient.

In another embodiment, the invention provides any of the above-described methods in which the sustained pre-selected amount of the interferon receptor agonist in the first sustained dosage interval is at least about 90%, or at least about 100%, and up to about 150%, of the initial pre-selected amount of the interferon receptor agonist, and the sustained pre-selected amount of the interferon receptor agonist of any following sustained dosage interval is at least about 100%, and up to about 150%, of the sustained pre-selected amount of the interferon receptor agonist in the preceding sustained dosage interval.

In another embodiment, the invention provides any of the above-described methods in which the sustained dosage phase consists of only two sustained dosage intervals (a first sustained dosage interval and a single following sustained dosage interval), the first sustained pre-selected amount of the interferon receptor agonist is about 100% of the initial pre-selected amount of the interferon receptor agonist, the initial dosage phase and the first sustained dosage interval extend for a combined period of time of about 4 weeks, and the sustained pre-selected amount of the interferon receptor agonist in the following sustained dosage interval is about 150% of the sustained pre-selected amount of the interferon receptor agonist in the first sustained dosage interval.

In another embodiment, the invention provides any of the above-described methods in which the sustained dosage phase consists of two sustained dosage intervals, and the sustained pre-selected amount of the interferon receptor agonist in the following sustained dosage interval is at least about 50% and up to about 70% of the sustained pre-selected amount of the interferon receptor agonist in the first sustained dosage interval. Optionally, the initial dosage phase and the first sustained dosage interval extend for a combined period of time of about 4 weeks.

In another embodiment, the invention provides any of the above-described methods in which the sustained dosage phase consists of three sustained dosage intervals, the sustained pre-selected amount of the interferon receptor agonist in the second sustained dosage interval (the first to occur of the following sustained dosage intervals) is at least about 60% and up to about 70% of the first sustained pre-selected amount of the interferon receptor agonist, the sustained pre-selected amount of the interferon receptor agonist in the third sustained dosage interval (the last to occur of the following sustained dosage intervals) is about 50% of the sustained pre-selected amount of the interferon receptor agonist in the second sustained dosage interval, the initial dosage phase and the first sustained dosage interval extend for a combined period of time of about 4 weeks, and the second sustained dosage interval extends for a period of time of about 8 weeks.

In another embodiment, the invention provides any of the above-described methods in which the interferon receptor agonist is an IFN-α, and the sustained pre-selected amount of the IFN-α in the last sustained dosage interval is at least about 0.5 million Units (MU), or at least about 1.0 MU, or at least about 1.5 MU, or at least about 2.0 MU, or at least about 2.5 MU, or at least about 3 MU, or at least about 6 MU, or at least about 9 MU, or at least about 12 MU, or at least about 15 MU, or at least about 18 MU, or at least about 21 MU, or at least about 24 MU, or at least about 27 MU, or at least about 30 MU, administered subcutaneously.

In another embodiment, the invention provides any of the above-described methods in which the interferon receptor agonist is a consensus interferon (CIFN), and the sustained pre-selected amount of the CIFN in the last sustained dosage interval is at least about 0.5 μg, or at least about 1.0 μg, or at least about 1.5 μg, or at least about 2.0 μg, or at least about 2.5 μg, or at least about 3 μg, or at least about 6 μg, or at least about 9 μg, or at least about 12 μg, or at least about 15 μg, or at least about 18 μg, or at least about 21 μg, or at least about 24 μg, or at least about 27 μg, or at least about 30 μg, of the CIFN administered subcutaneously.

In another embodiment, the invention provides any of the above-described methods in which the interferon receptor agonist is an IFN-α, and the initial pre-selected amount of the IFN-α is at least about 0.5 million Units (MU), or at least about 1.0 MU, or at least about 1.5 MU, or at least about 2.0 MU, or at least about 2.5 MU, or at least about 3 MU, or at least about 6 MU, or at least about 9 MU, or at least about 12 MU, or at least about 15 MU, or at least about 18 MU, or at least about 21 MU, or at least about 24 MU, or at least about 27 MU, or at least about 30 MU, administered subcutaneously.

In another embodiment, the invention provides any of the above-described methods in which the interferon receptor agonist is a consensus interferon (CIFN), and the initial pre-selected amount of the CIFN is at least about 0.5 μg, or at least about 1.0 μg, or at least about 1.5 μg, or at least about 2.0 μg, or at least about 2.5 μg, or at least about 3 μg, or at least about 6 μg, or at least about 9 μg, or at least about 12 μg, or at least about 15 μg, or at least about 18 μg, or at least about 21 μg, or at least about 24 μg, or at least about 27 μg, or at least about 30 μg, of the CIFN administered subcutaneously.

In another embodiment, the invention provides any of the above-described methods in which the sustained pre-selected amount of the interferon receptor agonist in the last sustained dosage interval is at least about 55%, or at least about 60%, or at least about 65%, or at least about 70%, or at least about 75%, or at least about 80%, or at least about 85%, or at least about 90%, or at least about 95%, and up to about 100%, of the maximum tolerated dose (MTD) of the patient.

In another embodiment, the invention provides any of the above-described methods in which the initial pre-selected amount of the interferon receptor agonist is at least about 55%, or at least about 60%, or at least about 65%, or at least about 70%, or at least about 75%, or at least about 80%, or at least about 85%, or at least about 90%, or at least about 95%, and up to about 100%, of the maximum tolerated dose (MID) of the patient.

In another embodiment; the invention provides any of the above-described methods in which the initial pre-selected amount of the interferon receptor agonist and every sustained pre-selected amount of the interferon receptor agonist are substantially the same.

The invention also features a modification of any of the above-described methods in which each period or phase of substantially continuous or continuous administration of interferon receptor agonist to the patient is altered to incorporate a sleep/wake dosing cycle that is repeated for the duration of any such period or phase in the subject method, where the sleep/wake dosing cycle delivers the majority of the daily dosage of the interferon receptor agonist to the patient in a substantially continuous or continuous manner during the patient's sleeping hours in any such period or phase. Optionally, the sleep/wake dosing cycle utilizes a pattern of 8 sleeping hours/16 waking hours, or 10 sleeping hours/14 waking hours, or 12 sleeping hours/12 waking hours, for a total of 24 hours in each cycle.

The invention also features a modification of any of the above-described methods in which each period or phase of substantially continuous or continuous administration of interferon receptor agonist to the patient is altered to incorporate a sleep/wake dosing cycle that is repeated for the duration of any such period or phase in the subject method, where the sleep/wake dosing cycle delivers at least about 50% of the daily dosage of the interferon receptor agonist as a bolus at the beginning or within about the first hour of the sleeping hours and the balance of the daily dosage is delivered substantially continuously or continuously during the waking hours for each 24 hour interval in any such period or phase. Optionally, the sleep/wake dosing cycle utilizes a pattern of 8 sleeping hours/16 waking hours, or 10 sleeping hours/14 waking hours, or 12 sleeping hours/12 waking hours, for a total of 24 hours in each cycle.

The invention also features a modification of any of the above-described methods in which each period or phase of substantially continuous or continuous administration of interferon receptor agonist to the patient is altered to incorporate a bolus pulse delivery cycle that is repeated for the duration of any such period or phase in the subject method, where the bolus pulse cycle provides three or more equal bolus administrations of the interferon receptor agonist that in the aggregate equal the total dosage of the interferon receptor agonist to be administered to the patient during each 24 hour span of time or fraction(s) thereof in which substantially continuous or continuous delivery of interferon receptor agonist would otherwise occur, and where the bolus administrations are separated by evenly spaced intervals of time in each bolus pulse delivery cycle. Optionally, the bolus pulse delivery cycle uses six bolus doses where the bolus doses are administered by an implantable infusion pump at 4 hour intervals during each bolus pulse delivery cycle.

In another embodiment, the invention provides any of the above-described methods in which a therapeutically effective amount of ribavirin is co-administered to the patient for the duration of the interferon receptor agonist therapy. Optionally, the method provides for administering to the patient about 800 mg to about 1200 mg ribavirin orally per day for the duration of the interferon receptor agonist therapy. Optionally, the method provides for administering to the patient (a) 1000 mg ribavirin orally per day if the patient has a body weight less than 75 kg or (b) 1200 mg ribavirin orally per day of the patient has a body weight greater than or equal to 75 kg, where the daily dosage of ribavirin is administered to the individual in 2 divided doses per day for the duration of the therapy.

The invention also features any of the above-described methods in which the duration of the interferon receptor agonist therapy is about 24 weeks. Optionally, the duration of the interferon receptor agonist therapy is about 48 weeks. Optionally, the duration of the interferon receptor agonist therapy is about 60 weeks.

In another aspect, the invention features any of the above-described methods in which the patient is an antiviral treatment naïve patient having a genotype I HCV infection and an initial viral load of greater than 2 million HCV RNA genome copies per ml of serum, and the duration of the interferon receptor agonist therapy is about 48 weeks.

In another aspect, the invention features any of the above-described methods in which the patient is an antiviral treatment naïve patient having a genotype 1 HCV infection and an initial viral load of less than or equal to 2 million HCV RNA genome copies per ml of serum, and the duration of the interferon receptor agonist therapy is about 24 weeks up to about 48 weeks.

In another aspect, the invention features any of the above-described methods in which the patient is an antiviral treatment naïve patient having a genotype 4 HCV infection, and the duration of the interferon receptor agonist therapy is about 48 weeks.

In another aspect, the invention features any of the above-described methods in which the patient is an antiviral treatment naïve patient having a genotype 2 or 3 HCV infection, and the duration of the interferon receptor agonist therapy is about 6 weeks to about 24 weeks.

In another aspect, the invention features any of the above-described methods in which the patient failed at least one earlier course of antiviral therapy for HCV infection and the duration of the interferon receptor agonist therapy is about 24 weeks to about 60 weeks.

In another aspect, the invention features any of the above-described methods in which the patient failed at least one earlier course of IFN-α monotherapy or IFN-α and ribavirin combination therapy for HCV infection, and the duration of the interferon receptor agonist therapy performed in the method is about 24 weeks to about 60 weeks. Optionally, the earlier course of IFN-α therapy was either IFN-α 2a or 2b therapy. Optionally, the earlier course of IFN-α therapy was either PEGASYS® peginterferon alfa-2a or PEG-INTRON® peginterferon alfa-2b therapy.

In another aspect, the invention features any of the above-described methods in which the interferon receptor agonist is a consensus interferon, the patient failed at least one earlier course of IFN-α monotherapy or IFN-α and ribavirin combination therapy for HCV infection, and the duration of the consensus interferon therapy performed in the method is about 24 weeks to about 60 weeks. Optionally, the earlier course of IFN-α therapy was either IFN-α 2a or 2b therapy. Optionally, the earlier course of IFN-α therapy was either PEGASYS® peginterferon alfa-2a or PEG-INTRON® peginterferon alfa-2b therapy. Optionally, the consensus interferon is INFERGEN® interferon alfacon-1.

In another aspect, the invention features any of the above-described methods in which the patient has a genotype 2 or 3 HCV infection and relapsed after responding to at least one earlier course of IFN-α monotherapy or IFN-α and ribavirin combination therapy for HCV infection, and the duration of the interferon receptor agonist therapy performed in the method is about 24 weeks to about 48 weeks. Optionally, the earlier course of IFN-α therapy was either IFN-α 2a or 2b therapy. Optionally, the earlier course of IFN-α therapy was either PEGASYS® peginterferon alfa-2a or PEG-INTRON® peginterferon alfa-2b therapy.

In another aspect, the invention features any of the above-described methods in which the interferon receptor agonist is a consensus interferon, the patient has a genotype 2 or 3 HCV infection and relapsed after responding to at least one earlier course of IFN-α monotherapy or IFN-α and ribavirin combination therapy for HCV infection, and the duration of the consensus interferon therapy performed in the method is about 24 weeks to about 48 weeks. Optionally, the earlier course of IFN-α therapy was either IFN-α 2a or 2b therapy. Optionally, the earlier course of IFN-α therapy was either PEGASYS® peginterferon alfa-2a or PEG-INTRON® peginterferon alfa-2b therapy. Optionally, the consensus interferon is INFERGEN® interferon alfacon-1.

In another aspect, the invention features any of the above-described methods in which the patient has a genotype 1 or 4 HCV infection and relapsed after responding to at least one earlier course of IFN-α monotherapy or IFN-α and ribavirin combination therapy for HCV infection, and the duration of the interferon receptor agonist therapy performed in the method is about 48 weeks. Optionally, the earlier course of IFN-α therapy was either IFN-α 2a or 2b therapy. Optionally, the earlier course of IFN-α therapy was either PEGASYS® peginterferon alfa-2a or PEG-INTRON® peginterferon alfa-2b therapy.

In another aspect, the invention features any of the above-described methods in which the interferon receptor agonist is a consensus interferon, the patient has a genotype 1 or 4 HCV infection and relapsed after responding to at least one earlier course of IFN-α monotherapy or IFN-α and ribavirin combination therapy for HCV infection, and the duration of the consensus interferon therapy performed in the method is about 48 weeks. Optionally, the earlier course of IFN-α therapy was either IFN-α 2a or 2b therapy. Optionally, the earlier course of IFN-α therapy was either PEGASYS® peginterferon alfa-2a or PEG-INTRON® peginterferon alfa-2b therapy. Optionally, the consensus interferon is INFERGEN® interferon alfacon-1.

In another aspect, the invention features any of the above-described methods in which the patient did not respond to at least one earlier course of IFN-α monotherapy or IFN-α and ribavirin combination therapy for HCV infection, and the duration of the interferon receptor agonist therapy performed in the method is about 48 weeks to about 60 weeks. Optionally, the earlier course of IFN-α therapy was either IFN-α 2a or 2b therapy. Optionally, the earlier course of IFN-α therapy was either PEGASYS® peginterferon alfa-2a or PEG-INTRON® peginterferon alfa-2b therapy.

In another aspect, the invention features any of the above-described methods in which the interferon receptor agonist is a consensus interferon, the patient did not respond to at least one earlier course of IFN-α monotherapy or IFN-α and ribavirin combination therapy for HCV infection, and the duration of the interferon receptor agonist therapy performed in the method is about 48 weeks to about 60 weeks. Optionally, the earlier course of IFN-α therapy was either IFN-α 2a or 2b therapy. Optionally, the earlier course of IFN-α therapy was either PEGASYS® peginterferon alfa-2a or PEG-INTRON® peginterferon alfa-2b therapy. Optionally, the consensus interferon is INFERGEN® interferon alfacon-1.

In another aspect, the invention features any of the above-described methods in which before the initial administration of interferon receptor agonist (a) the patient is identified as an antiviral treatment naïve patient having a genotype 1 HCV infection and an initial viral load of greater than 2 million HCV RNA genome copies/ml of serum and the duration of interferon receptor agonist therapy is set at about 48 weeks (b) the patient is identified as an antiviral treatment naïve patient having a genotype 1 HCV infection and an initial viral load of less than or equal to 2 million HCV RNA genome copies/ml of serum and the duration of interferon receptor agonist therapy is set at about 24 weeks to about 48 weeks (c) the patient is identified as an antiviral treatment naïve patient having a genotype 2 or 3 HCV infection and the duration of the interferon receptor agonist therapy is set at about 6 weeks to about 24 weeks (d) the patient is identified as an antiviral treatment naïve patient having a genotype 4 HCV infection and the duration of the interferon receptor agonist therapy is set at about 48 weeks (e) the patient is identified as having relapsed after responding to an earlier course of IFN-α therapy and as having a genotype 1 or 4 HCV infection and the duration of interferon receptor agonist therapy is set at about 48 weeks (f) the patient is identified as having relapsed after responding to an earlier course of IFN-α therapy and as having a genotype 2 or 3 HCV infection and the duration of interferon receptor agonist therapy is set at about 24 weeks to about 48 weeks or (g) the patient is identified as having failed to respond to an earlier course of IFN-® therapy and the duration of interferon receptor agonist therapy is set at about 48 weeks to about 60 weeks.

In another aspect, the invention features any of the above-described methods specific for the antiviral treatment history of the patient, the genotype of the HCV infection of the patient, and/or the initial viral load of the patient, in which the interferon receptor agonist is a Type I interferon receptor agonist. In some embodiments, the Type I interferon receptor agonist is an IFN-α. Optionally, the IFN-α is a consensus interferon.

In another aspect, the invention features any of the above-described methods specific for the antiviral treatment history of the patient, the genotype of the HCV infection of the patient, and/or the initial viral load of the patient, in which a therapeutically effective amount of ribavirin is also administered to the patient for the duration of the interferon receptor agonist therapy. Optionally, the method provides administering to the patient about 800 mg to about 1200 mg ribavirin orally per day for the duration of the interferon receptor agonist therapy. Optionally, the method provides for administering to the patient (a) 1000 mg ribavirin orally per day if the patient has a body weight less than 75 kg or (b) 1200 mg ribavirin orally per day if the patient has a body weight greater than or equal to 75 kg, where the daily dosage of ribavirin is administered to the individual in 2 divided doses per day for the duration of the interferon receptor agonist therapy.

In another aspect, the invention features any of the above-described methods in which the interferon receptor agonist is an unPEGylated IFN-α. Optionally, the unPEGylated IFN-α is an unPEGylated consensus interferon.

In another aspect, the invention features any of the above-described methods in which the interferon receptor agonist is an IFN-α and the subject method further comprises co-administering to the patient an effective amount of IFN-γ for the duration of the IFN-α therapy. In one embodiment, the IFN-γ is administered to the patient by bolus injection. In another embodiment, the IFN-α and IFN-γ are administered to the patient by a drug delivery device. Optionally, the device is used to deliver the IFN-α to the patient by substantially continuous or continuous administration and used to deliver the IFN-γ to the patient by bolus administration tiw, biw, qod, or qd. Optionally, the device is used to deliver the IFN-α and IFN-γ to the patient in the same manner and pattern of administration, such as substantially continuous or continuous administration. Optionally, the IFN-α and IFN-γ are contained in separate reservoirs in the drug delivery device. Optionally, the IFN-α and IFN-γ are co-formulated in a single liquid formulation that is contained in a single reservoir in the drug delivery device.

In another aspect, the invention features any of the above-described methods in which the subject method further comprises co-administering to the patient an effective amount of pirfenidone or a pirfenidone analog orally qd, optionally in two or more divided doses per day, for the duration of the interferon receptor agonist therapy.

In another aspect, the invention features any of the above-described methods in which the subject method further comprises co-administering to the patient for the duration of the interferon receptor agonist therapy provided in the subject method an amount of pirfenidone or a pirfenidone analog that is synergistically effective with the interferon receptor agonist therapy.

In another aspect, the invention features any of the above-described methods in which the subject method further comprises co-administering to the patient for the duration of the interferon receptor agonist therapy provided in the subject method an amount of pirfenidone or pirfenidone analog that is effective to reduce side effects induced by the interferon receptor agonist therapy.

In another aspect, the invention features any of the above-described methods in which the interferon receptor agonist is a Type I interferon receptor agonist and the subject method further comprises co-administering to the patient an effective amount of IFN-γ and an effective amount of pirfenidone or a pirfenidone analog for the duration of the interferon receptor agonist therapy. Optionally, the Type I interferon receptor agonist is an IFN-α. Optionally, the IFN-α is a consensus interferon.

In another embodiment, the invention features any of the above-described methods in which the interferon receptor agonist is a Type I interferon receptor agonist and the subject method further comprises co-administering to the patient for the duration of the Type I interferon receptor agonist therapy provided in the subject method an amount of IFN-γ and an amount of pirfenidone or a pirfenidone analog that are synergistically effective with the Type I interferon receptor agonist therapy. Optionally, the Type I interferon receptor agonist is an IFN-α. Optionally, the IFN-α is a consensus interferon.

In another aspect, the invention features any of the above-described methods in which the interferon receptor agonist is a Type I interferon receptor agonist and the subject method further comprises co-administering to the patient for the duration of the Type I interferon receptor agonist therapy provided in the subject method an amount of IFN-γ that increases the effectiveness of the Type I interferon receptor agonist therapy and an amount of pirfenidone or pirfenidone analog that reduces side effects induced by the Type I interferon receptor agonist and/or IFN-γ therapies. Optionally, the amount of the IFN-γ synergistically increases the efficacy of the Type I interferon receptor agonist therapy. Optionally, the Type I interferon receptor agonist is an IFN-α. Optionally, the IFN-α is a consensus interferon.

The present invention also features an apparatus designed for the administration of an interferon receptor agonist to a patient having an HCV infection according any of the methods described herein.

In one aspect, the invention provides an apparatus for administering an interferon receptor agonist to a patient having an HCV infection, where the apparatus includes (a) a device for the delivery of an interferon receptor agonist to a patient and (b) a control unit operated by a series of commands comprising a set of instructions that causes the device to administer to the patient a therapeutically effective amount of the interferon receptor agonist according to any of the methods described herein, where the control unit executes the set of instructions in the series of commands after the apparatus is installed on the patient, armed for operation, and activated to administer the interferon receptor agonist to the patient.

In another aspect, the invention provides an apparatus for administering an interferon receptor agonist to a patient having an HCV infection, where the apparatus includes (i) a device for delivery of an interferon receptor agonist to the patient by a selected route of administration and (ii) a control unit operated by a series of commands, where the series of commands contains a set of instructions that causes the device to administer to the patient a therapeutically effective amount of the interferon receptor agonist by the selected route of administration in a manner effective to achieve and maintain a sustained serum concentration of the interferon receptor agonist at a substantially steady state for a treatment period of at least about 6 weeks, or at least about 12 weeks, or at least about 24 weeks, or at least about 48 weeks, or at least about 60 weeks, and where the control unit executes the set of instructions in the series of commands after the apparatus is installed on the patient, armed for operation, and activated to administer the interferon receptor agonist to the patient. Optionally, the sustained serum concentration of the interferon receptor agonist is at least about 55%, or at least about 60%, or at least about 65%, or at least about 70%, or at least about 75%, or at least about 80%, or at least about 85%, or at least about 90%, or at least about 95%, and up to about 100%, of the maximum tolerated dose (MTD) of the patient. Optionally, the interferon receptor agonist is delivered to the patient subcutaneously. Optionally, the device is an implantable infusion pump and the set of instructions provides for administering the interferon receptor agonist to the patient via subcutaneous infusion in a substantially continuous or continuous manner by the infusion pump.

In another aspect, the invention provides an apparatus for administering an interferon receptor agonist to a patient having an HCV infection, where the apparatus includes (i) a device for delivery of an interferon receptor agonist to the patient by a selected route of administration and (ii) a control unit operated by a series of commands, where the series of commands contains a set of instructions that causes the device to administer to the patient a therapeutically effective amount of the interferon receptor agonist by the selected route of administration for a treatment period of at least about 6 weeks, or at least about 12 weeks, or at least about 24 weeks, or at least about 48 weeks, or at least about 60 weeks, in a manner effective to achieve an area under the curve of serum concentration of the interferon receptor agonist over time for any 8 hour interval in the treatment period (AUC₈ₕᵣ) that is no more than about 20% above or about 20% below, or no more than about 15% above or about 15% below, or no more than about 10% above or about 10% below, or no more than about 5% above or about 5% below, an average area under the curve of serum concentration of the interferon receptor agonist over time for an 8 hour interval in the treatment period (AUC_{8hr average}), where the AUC_{8hr average} is equal to the quotient of the area under the curve of serum concentration of the interferon receptor agonist over the entirety of the treatment period (AUCₜₒₜₐₗ) divided by the number of 8 hour intervals in the treatment period (t_{total1/3days}) and where the control unit executes the set of instructions after the apparatus is installed on the patient, armed for operation, and activated to administer the interferon receptor agonist to the patient.

In one embodiment, the invention provides the above-described apparatus in which the set of instructions provides for an area under the curve of serum concentration of the interferon receptor agonist over time for any 4 hour interval in the treatment period (AUC₄ₕᵣ) that is no more than about 20% above or about 20% below, or no more than about 15% above or about 15% below, or no more than about 10% above or about 10% below, or no more than about 5% above or about 5% below, an average area under the curve of serum concentration of the interferon receptor agonist over time for a 4 hour interval in the treatment period (AUC_{4hr average}), where the AUC_{4hr average} is equal to the quotient of the area under the curve of serum concentration of the interferon receptor agonist over the entirety of the treatment period (AUCₜₒₜₐₗ) divided by the number of 4 hour intervals in the treatment period (t_{total1/6days}).

In another embodiment, the invention provides the above-described apparatus in which the set of instructions provides for an area under the curve of serum concentration of the interferon receptor agonist over time for any 3 hour interval in the treatment period (AUC₃ₕᵣ) that is no more than about 20% above or about 20% below, or no more than about 15% above or about 15% below, or no more than about 10% above or about 10% below, or no more than about 5% above or about 5% below, an average area under the curve of serum concentration of the interferon receptor agonist over time for a 3 hour interval in the treatment period (AUC_{3hr average}) where the AUC_{3hr average} is equal to the quotient of the area under the curve of serum concentration of the interferon receptor agonist over the entirety of the treatment period (AUCₜₒₜₐₗ) divided by the number of 3 hour intervals in the treatment period (t_{total1/8days}).

In another embodiment, the invention provides the above-described apparatus in which the set of instructions provides for an area under the curve of serum concentration of the interferon receptor agonist over time for any 2 hour interval in the treatment period (AUC₂ₕᵣ) that is no more than about 20% above or about 20% below, or no more than about 15% above or about 15% below, or no more than about 10% above or about 10% below, or no more than about 5% above or about 5% below, an average area under the curve of serum concentration of the interferon receptor agonist over time for a 2 hour interval in the treatment period (AUC_{2hr average}), where the AUC_{2hr average} is equal to the quotient of the area under the curve of serum concentration of the interferon receptor agonist over the entirety of the treatment period (AUCₜₒₜₐₗ) divided by the number of 2 hour intervals in the treatment period (t_{total1/12days}).

In another embodiment, the invention provides the above-described apparatus in which the set of instructions provides for an area under the curve of serum concentration of the interferon receptor agonist over time for any 1 hour interval in the treatment period (AUC₁ₕᵣ) that is no more than about 20% above or about 20% below, or no more than about 15% above or about 15% below, or no more than about 10% above or about 10% below, or no more than about 5% above or about 5% below, an average area under the curve of serum concentration of the interferon receptor agonist over time for a 1 hour interval in the treatment period (AUC_{1hr average}), where the AUC_{1hr average} is equal to the quotient of the area under the curve of serum concentration of the interferon receptor agonist over the entirety of the treatment period (AUCₜₒₜₐₗ) divided by the number of hours in the treatment period (tₜₒₜₐₗₕᵣₛ).

In another embodiment, the set of instructions provides for an AUC_{8hr average}, or an AUC_{4hr average}, or an AUC_{3hr average}, or an AUC_{2hr average}, or an AUC_{1hr average}, that is at least about 55%, or at least about 60%, or at least about 65%, or at least about 70%, or at least about 75%, or at least about 80%, or at least about 85%, or at least about 90%, or at least about 95%, and up to about 100%, of the maximum tolerated dose (MTD) of the patient.

In another embodiment, the device is an implantable infusion device and the set of instructions provides for administering the interferon receptor agonist to the patient via subcutaneous infusion in a substantially continuous or continuous manner by the infusion pump.

In another aspect, the invention provides an apparatus for administering an interferon receptor agonist to a patient having an HCV infection, where the apparatus includes (i) a device for delivery of an interferon receptor agonist to the patient by a selected route of administration and (ii) a control unit operated by a series of commands, where the series of commands contains a set of instructions that causes the device to administer to the patient a therapeutically effective amount of the interferon receptor agonist by the selected route of administration in an initial dosage phase followed by a sustained dosage phase consisting of at least one sustained dosage interval, where during the initial dosage phase an initial serum concentration of the interferon receptor agonist is achieved within a period of time of about 12 hours to about 48 hours, where during the first sustained dosage interval a first sustained serum concentration of the interferon receptor agonist is achieved and maintained at a substantially steady state for a period of time of at least about 5 days, where the first sustained serum concentration of the interferon receptor agonist is at least about 80% and up to about 200% of the initial serum concentration of the interferon receptor agonist, and during any following sustained dosage interval a following sustained serum concentration of the interferon receptor agonist is achieved and maintained at a substantially steady state for period of time of at least about 5 days, where the following sustained serum concentration of the interferon receptor agonist is at least about 20% of the first sustained serum concentration of the interferon receptor agonist and at least about 50% and up to about 200% of the sustained serum concentration of the interferon receptor agonist in the preceding sustained dosage interval, where the duration of the interferon receptor agonist therapy is at least about 6 weeks, or at least about 12 weeks, or at least about 24 weeks, or at least about 48 weeks, or at least about 60 weeks, and where the control unit executes the set of instructions in the series of commands after the apparatus is installed on the patient, armed for operation, and activated to administer the interferon receptor agonist to the patient.

In one embodiment, the set of instructions provides for a sustained serum concentration of the interferon receptor agonist in every following sustained dosage interval that is at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 100%, of the serum concentration of the interferon receptor agonist in the first sustained dosage interval.

In another embodiment, the apparatus of the invention has a set of instructions that provides for administering interferon receptor agonist to the patient in a substantially continuous or continuous manner during at least the sustained dosage phase.

In another embodiment, the apparatus of the invention has a device that is an implantable infusion pump and a set of instructions that provides for administering interferon receptor agonist to the patient via subcutaneous infusion in a substantially continuous or continuous manner by the infusion pump during at least the sustained dosage phase. Optionally, the set of instructions causes the implantable infusion pump to (i) administer to the patient a single bolus dose of the interferon receptor agonist to achieve the initial serum concentration during the initial dosage phase and (ii) administer to the patient a pre-selected amount of the interferon receptor agonist per day by substantially continuous or continuous infusion to achieve and maintain the sustained serum concentration for each sustained dosage interval. Optionally, the interferon receptor agonist is an IFN-α, the implantable infusion pump is installed for subcutaneous delivery of the IFN-α, the bolus dose is at least about 0.5 million Units (MU), or at least about 1.5 MU, or at least about 2.0 MU, or at least about 2.5 MU, or at least about 3 MU of the IFN-α, and/or the pre-selected amount of the IFN-α is at least about 0.5 million Units (MU), or at least about 1.5 MU, or at least about 2.0 MU, or at least about 2.5 MU, or at least about 3 MU per day.

In another embodiment, the apparatus of the invention has a set of instructions that provides for administering interferon receptor agonist to the patient in a substantially continuous or continuous manner during the initial and sustained dosage phases. Optionally, the device is an implantable infusion pump and the set of instructions provides for administering the interferon receptor agonist to the patient via subcutaneous infusion in a substantially continuous or continuous manner by the infusion pump during the initial and sustained dosage phases. Optionally, the set of instructions causes the implantable infusion pump to deliver a pre-selected amount of the interferon receptor agonist per day to achieve the initial serum concentration of the interferon receptor agonist during the initial dosage phase and to achieve and maintain the sustained serum concentration of the interferon receptor agonist in each sustained dosage interval. Optionally, the interferon receptor agonist is an IFN-α, the implantable infusion pump is installed for subcutaneous delivery of the IFN-α, and the pre-selected amount is at least about 0.5 million Units (MU), or at least about 1.5 MU, or at least about 2.0 MU, or at least about 2.5 MU, or at least about 3 MU of the IFN-α per day and is administered to the patient by subcutaneous infusion.

In another embodiment, the invention provides any of the above-described apparatus in which the set of instructions provides for a sustained serum concentration of the interferon receptor agonist in the first sustained dosage interval that is at least about 85%, or at least about 90%, or at least about 95%, or at least about 100%, of the initial serum concentration of the interferon receptor agonist. Optionally, the set of instructions provides for a sustained dosage phase consisting of a single sustained dosage interval.

In another embodiment, the invention provides any of the above-described apparatus in which the set of instructions provides for an initial serum concentration of the interferon receptor agonist that is substantially the same as the sustained serum concentration of the interferon receptor agonist in the first sustained dosage interval. Optionally, the set of instructions provides for a sustained dosage phase consisting of a single sustained dosage interval.

In another embodiment, the invention provides any of the above-described apparatus in which the set of instructions provides for an initial serum concentration of the interferon receptor agonist that is at least about 55%, or at least about 60%, or at least about 65%, or at least about 70%, or at least about 75%, or at least about 80%, or at least about 85%, or at least about 90%, or at least about 95%, and up to about 100%, of the maximum tolerated dose (MID) of the patient.

In another embodiment, the invention provides any of the above-described apparatus in which the set of instructions provides for more than one sustained dosage interval and the sustained serum concentration of the interferon receptor agonist in the last sustained dosage interval is at least about 55%, or at least about 60%, or at least about 65%, or at least about 70%, or at least about 75%, or at least about 80%, or at least about 85%, or at least about 90%, or at least about 95%, and up to about 100%, of the maximum tolerated dose (MTD) of the patient. Optionally, the sustained serum concentration of the interferon receptor agonist in the first sustained dosage interval is at least about 90%, or at least about 100%, and up to about 150%, of the initial serum concentration of the interferon receptor agonist, and the sustained serum concentration in any following sustained dosage interval is at least about 100%, and up to about 150%, of the sustained serum concentration of the interferon receptor agonist in the preceding sustained dosage interval.

In another embodiment, the invention provides any of the above-described apparatus in which the sustained dosage phase consists of only two sustained dosage intervals (the first sustained dosage interval and a single following sustained dosage interval), the sustained serum concentration of the interferon receptor agonist in the first sustained dosage interval is about 100% of the initial serum concentration of the interferon receptor agonist, the initial dosage phase and the first sustained dosage interval extend for a combined period of time about 4 weeks, and the sustained serum concentration of the interferon receptor agonist is about 150% of the sustained serum concentration of the interferon receptor agonist in the first sustained dosage interval.

In another embodiment, the invention provides any of the above-described apparatus in which the sustained dosage phase consists of two or three sustained dosage intervals, and the sustained serum concentration of the interferon receptor agonist in each following sustained dosage interval is at least about 50% and up to about 70% of the serum concentration in the preceding sustained dosage interval.

In another embodiment, the invention provides any of the above-described apparatus in which the sustained dosage phase consists of two sustained dosage intervals, and the sustained serum concentration of the interferon receptor agonist in the following sustained dosage interval is at least about 50% and up to about 70% of the sustained serum concentration of the interferon receptor agonist in the first sustained dosage interval. Optionally, the initial dosage phase and the first sustained dosage interval extend for a combined period of time of about 4 weeks.

In another embodiment, the invention provides any of the above-described apparatus in which the sustained dosage phase consists of three sustained dosage intervals, the sustained serum concentration of the interferon receptor agonist in the second sustained dosage interval (the first to occur of the following sustained dosage intervals) is at least about 60% and up to about 70% of the first sustained serum concentration of the interferon receptor agonist, the sustained serum concentration of the interferon receptor agonist in the third sustained dosage interval (the last to occur of the following sustained dosage intervals) is about 50% of the sustained serum concentration of the interferon receptor agonist in the second sustained dosage interval, the initial dosage phase and the first sustained dosage interval extend for a combined period of time of about 4 weeks, and the second sustained dosage interval extends for a period of time of about 8 weeks.

In another embodiment, the invention provides any of the above-described apparatus in which the set of instructions provides for an initial dosage phase that extends for a period of time of about 24 hours.

In another embodiment, the invention provides any of the above-described apparatus in which the set of instructions provides in each sustained dosage interval an area under the curve of serum concentration of the interferon receptor agonist over time for any 8 hour period in the sustained dosage interval (AUC₈ₕᵣ) that is no more than about 20% above or about 20% below, or no more than about 15% above or about 15% below, or no more than about 10% above or about 10% below, or no more than about 5% above or about 5% below, an average area under the curve of serum concentration of the interferon receptor agonist over time for an 8 hour period in the sustained dosage interval (AUC_{8hr average}), where the AUC_{8hr average} is equal to the quotient of the area under the curve of serum concentration of the interferon receptor agonist for the entirety of the sustained dosage interval (AUCₜₒₜₐₗ) divided by the number of 8 hour periods in the sustained dosage interval (t_{total1/3days}), i.e., the AUC_{8hr average}=AUC_{total1/3days}.

In another embodiment, the invention provides any of the above-described apparatus in which the set of instructions provides in each sustained dosage interval an area under the curve of serum concentration of the interferon receptor agonist over time for any 4 hour period in the sustained dosage interval (AUC₄ₕᵣ) that is no more than about 20% above or about 20% below, or no more than about 15% above or about 15% below, or no more than about 10% above or about 10% below, or no more than about 5% above or about 5% below, an average area under the curve of serum concentration of the interferon receptor agonist over time for a 4 hour period in the sustained dosage interval (AUC_{4hr average}), where the AUC_{4hr average} is equal to the quotient of the area under the curve of serum concentration of the interferon receptor agonist for the entirety of the sustained dosage interval (AUCₜₒₜₐₗ) divided by the number of 4 hour periods in the sustained dosage interval (t_{total1/6days}), i.e., the AUC_{4hr average}=AUC_{total1/6days}.

In another embodiment, the invention provides any of the above-described apparatus in which the set of instructions provides in each sustained dosage interval an area under the curve of serum concentration of the interferon receptor agonist over time for any 3 hour period in the sustained dosage interval (AUC₃ₕᵣ) that is no more than about 20% above or about 20% below, or no more than about 15% above or about 15% below, or no more than about 10% above or about 10% below, or no more than about 5% above or about 5% below, an average area under the curve of serum concentration of the interferon receptor agonist over time for a 3 hour period in the sustained dosage interval (AUC_{3hr average}), where the AUC_{3hr average} is equal to the quotient of the area under the curve of serum concentration of the interferon receptor agonist for the entirety of the sustained dosage interval (AUCₜₒₜₐₗ) divided by the number of 3 hour periods in the sustained dosage interval (t_{total1/8days}), i.e., the AUC_{3hr average}=AUCₜₒₜₐₗ/t_{total1/8days}.

In another embodiment, the invention provides any of the above-described apparatus in which the set of instructions provides in each sustained dosage interval an area under the curve of serum concentration of the interferon receptor agonist over time for any 2 hour period in the sustained dosage interval (AUC₂ₕᵣ) that is no more than about 20% above or about 20% below, or no more than about 15% above or about 15% below, or no more than about 10% above or about 10% below, or no more than about 5% above or about 5% below, an average area under the curve of serum concentration of the interferon receptor agonist over time for a 2 hour period in the sustained dosage interval (AUC_{2hr average}) where the AUC_{2hr average} is equal to the quotient of the area under the curve of serum concentration of the interferon receptor agonist for the entirety of the sustained dosage interval (AUCₜₒₜₐₗ) divided by the number of 2 hour periods in the sustained dosage interval (t_{total1/12days}), i.e., the AUC_{2hr average}=AUCₜₒₜₐₗ/t_{total1/12days}.

In another embodiment, the invention provides any of the above-described apparatus in which the set of instructions provides in each sustained dosage interval an area under the curve of serum concentration of the interferon receptor agonist over time for any 1 hour period in the sustained dosage interval (AUC₁ₕᵣ) that is no more than about 20% above or about 20% below, or no more than about 15% above or about 15% below, or no more than about 10% above or about 10% below, or no more than about 5% above or about 5% below, an average area under the curve of serum concentration of the interferon receptor agonist over time for a 1 hour period in the sustained dosage interval (AUC_{1h average}), where the AUC_{1hr average} is equal to the quotient of the area under the curve of serum concentration of the interferon receptor agonist for the entirety of the sustained dosage interval (AUCₜₒₜₐₗ) divided by the number of hours in the sustained dosage interval (tₜₒₜₐₗₕᵣₛ), i.e., the AUC_{1hr average}=AUCₜₒₜₐₗ/tₜₒₜₐₗₕᵣₛ.

In some embodiments, the invention provides any one of the above-described apparatus in which the interferon receptor agonist is a Type I interferon receptor agonist. In other embodiments, the invention provides any one of the above-described apparatus in which the interferon receptor agonist is a Type II interferon receptor agonist. In other embodiments, the invention provides any one of the above-described apparatus in which the interferon receptor agonist is a Type III interferon receptor agonist.

In another embodiment, the invention provides any of the above-described apparatus in which the interferon receptor agonist is an IFN-α. Optionally, the IFN-α is a consensus interferon. Optionally, the consensus interferon is INFERGEN® interferon alfacon-1. In another embodiment, the invention provides any of the above-described apparatus in which the IFN-α is IFN-α 2a or IFN-α 2b.

In other embodiments, the invention provides any one of the above-described apparatus in which the interferon receptor agonist is an IFN-β. In other embodiments, the invention provides any one of the above-described apparatus in which the interferon receptor agonist is IFN-tau. In other embodiments, the invention provides any one of the above-described apparatus in which the interferon receptor agonist is IFN-ω.

In other embodiments, the invention provides any one of the above-described apparatus in which the interferon receptor agonist is an IFN-γ.

In another aspect, the apparatus of the invention includes (i) a device for delivery of an interferon receptor agonist to the patient by a selected route of administration and (ii) a control unit operated by a series of commands, where the series of commands contains a set of instructions that causes the device to administer to the patient a therapeutically effective amount of the interferon receptor agonist via the selected route of administration by substantially continuous or continuous delivery of a pre-selected amount of the interferon receptor agonist each day for a treatment period of at least about 6 weeks, or at least about 12 weeks, or at least about 24 weeks, or at least about 48 weeks, or at least about 60 weeks, where the control unit executes the set of instructions in the series of commands after the apparatus is installed on the patient, armed for operation, and activated to administer the interferon receptor agonist to the patient.

In one embodiment, the set of instructions provides for a pre-selected amount of the interferon receptor agonist per day that is at least about 55%, or at least about 60%, or at least about 65%, or at least about 70%, or at least about 75%, or at least about 80%, or at least about 85%, or at least about 90%, or at least about 95%, and up to about 100%, of the maximum tolerated dose (MTD) of the patient.

In another embodiment, the device is an implantable infusion pump and the set of instructions provides for administering the interferon receptor agonist via subcutaneous infusion to the patient in a substantially continuous or continuous manner by the infusion pump.

In another aspect, the invention provides an apparatus for administering an interferon receptor agonist to a patient having an HCV infection, where the apparatus includes (i) a device for delivery of an interferon receptor agonist to the patient by a selected route of administration and (ii) a control unit operated by a series of commands, where the series of commands contains a set of instructions that causes the device to administer to the patient a therapeutically effective amount of the interferon receptor agonist via the selected route of administration for a treatment period of at least about 6 weeks, or at least about 12 weeks, or at least about 24 weeks, or at least about 48 weeks, or at least about 60 weeks, where each day of the treatment period the patient receives by substantially continuous or continuous delivery an amount of the interferon receptor agonist that is no more than about 20% above or about 20% below, or no more than about 15% above or about 15% below, or no more than about 10% above or about 10% below, or no more than about 5% above or about 5% below, an average daily dosage of the interferon receptor agonist (ADD_{INFRa}), where the ADD is equal to the aggregate amount of the interferon receptor agonist administered to the patient in the treatment period divided by the number of days in the treatment period.

In another aspect, the invention provides an apparatus for administering an interferon receptor agonist to a patient having an HCV infection, where the apparatus includes (i) a device for delivery of the interferon receptor agonist to the patient by a selected route of administration and (ii) a control unit operated by a series of commands, where the series of commands contains a set of instructions that causes the device to administer to the patient a therapeutically effective amount of the interferon receptor agonist via the selected route of administration in an initial dosage phase followed by a sustained dosage phase consisting of at least one sustained dosage interval, where the initial dosage phase extends for a period of time of about 12 hours to about 48 hours and during the initial dosage phase an initial pre-selected amount of the interferon receptor agonist is administered to the individual, where during the first sustained dosage interval a first sustained pre-selected amount of the interferon receptor agonist is administered to the individual each day in a substantially continuous or continuous manner, where the first sustained pre-selected amount of the interferon receptor agonist is at least about 80% and up to about 200% of the initial pre-selected amount of the interferon receptor agonist, and during any following sustained dosage interval a following sustained pre-selected amount of the interferon receptor agonist is administered to the individual each day in a substantially continuous or continuous manner, where the following sustained pre-selected amount of the interferon receptor agonist is at least about 20% of the first sustained pre-selected amount of the interferon receptor agonist and at least about 50% and up to about 200% of the sustained pre-selected amount of the interferon receptor agonist in the preceding sustained dosage interval, where each sustained dosage interval extends for a period of time of at least about 5 days, where the duration of the interferon receptor agonist therapy is at least about 6 weeks, or at least about 12 weeks, or at least about 24 weeks, or at least about 48 weeks, or at least about 60 weeks, and where the control unit executes the set of instructions in the series of commands after the apparatus is installed on the patient, armed for operation, and activated to administer the interferon receptor agonist to the patient.

In one embodiment, the apparatus of the invention has a set of instructions that provides in each sustained dosage interval an area under the curve of serum concentration of the interferon receptor agonist over time for any 8 hour period in the sustained dosage interval that is no more than about 20% above or about 20% below, or no more than about 15% above or about 15% below, or no more than about 10% above or about 10% below, or no more than about 5% above or about 5% below, an average area under the curve (AUC_{8hr average}), where the AUC_{8hr average} is equal to the quotient of the area under the curve of serum concentration of the interferon receptor agonist over the entirety of the sustained dosage interval (AUCₜₒₜₐₗ) divided by the number of 8 hour periods in the sustained dosage interval (t_{total1/3days}).

In another embodiment, the apparatus of the invention has a set of instructions that provides in each sustained dosage interval an area under the curve of serum concentration of the interferon receptor agonist over time for any 4 hour period in the sustained dosage interval that is no more than about 20% above or about 20% below, or no more than about 15% above or about 15% below, or no more than about 10% above or about 10% below, or no more than about 5% above or about 5% below, an average area under the curve (AUC_{4hr average}), where the AUC_{4hr average} is equal to the quotient of the area under the curve of serum concentration of the interferon receptor agonist over the entirety of the sustained dosage interval (AUCₜₒₜₐₗ) divided by the number of 4 hour periods in the sustained dosage interval (t_{total1/6days}).

In another embodiment, the apparatus of the invention has a set of instructions that provides in each sustained dosage interval an area under the curve of serum concentration of the interferon receptor agonist over time for any 3 hour period in the sustained dosage interval that is no more than about 20% above or about 20% below, or no more than about 15% above or about 15% below, or no more than about 10% above or about 10% below, or no more than about 5% above or about 5% below, an average area under the curve (AUC_{3hr average}), where the AUC_{3hr average} is equal to the quotient of the area under the curve of serum concentration of the interferon receptor agonist over the entirety of the sustained dosage interval (AUCₜₒₜₐₗ) divided by the number of 3 hour periods in the sustained dosage interval (t_{total1/8days}).

In another embodiment, the apparatus of the invention has a set of instructions that provides in each sustained dosage interval an area under the curve of serum concentration of the interferon receptor agonist over time for any 2 hour period in the sustained dosage interval that is no more than about 20% above or about 20% below, or no more than about 15% above or about 15% below, or no more than about 10% above or about 10% below, or no more than about 5% above or about 5% below, an average area under the curve (AUC_{2hr average}), where the AUC_{2hr average} is equal to the quotient of the area under the curve of serum concentration of the interferon receptor agonist over the entirety of the sustained dosage interval (AUCₜₒₜₐₗ) divided by the number of 2 hour periods in the sustained dosage interval (t_{total1/12days}).

In another embodiment, the apparatus of the invention has a set of instructions that provides in each sustained dosage interval an area under the curve of serum concentration of the interferon receptor agonist over time for any 1 hour period in the sustained dosage interval that is no more than about 20% above or about 20% below, or no more than about 15% above or about 15% below, or no more than about 10% above or about 10% below, or no more than about 5% above or about 5% below, an average area under the curve (AUC_{1hr average}) where the AUC_{1hr average} is equal to the quotient of the area under the curve of serum concentration of the interferon receptor agonist over the entirety of the sustained dosage interval (AUCₜₒₜₐₗ) divided by the number of hours in the sustained dosage interval (tₜₒₜₐₗₕᵣₛ).

In another embodiment, the apparatus of the invention has a set of instructions that provides for administering the interferon receptor agonist to the patient in a substantially continuous or continuous manner during the initial dosage phase and the sustained dosage phase. Optionally, the interferon receptor agonist is delivered to the patient by subcutaneous administration.

In another embodiment, the apparatus of the invention has a device that is an implantable infusion pump and has a set of instructions that provides for administering the interferon receptor agonist to the patient via subcutaneous infusion in a substantially continuous or continuous manner by the infusion pump during the sustained dosage phase. Optionally, the pump is implanted during the initial dosage phase and the set of instructions causes the pump to administer the initial pre-selected amount of the interferon receptor agonist as a bolus at the beginning of the initial dosage phase.

In another embodiment, the invention provides any of the above-described apparatus in which the device is installed to deliver the interferon receptor agonist to the patient by subcutaneous infusion during the initial and sustained dosage phases.

In some embodiments, the invention provides any one of the above-described apparatus in which the interferon receptor agonist is a Type I interferon receptor agonist. In other embodiments, the invention provides any one of the above-described apparatus in which the interferon receptor agonist is a Type II interferon receptor agonist. In other embodiments, the invention provides any one of the above-described apparatus in which the interferon receptor agonist is a Type III interferon receptor agonist.

In another embodiment, the invention provides any of the above-described apparatus in which the interferon receptor agonist is an IFN-α. Optionally, the IFN-α is a consensus interferon. Optionally, the consensus interferon is INFERGEN® interferon alfacon-1. In another embodiment, the invention provides any of the above-described apparatus in which the interferon receptor agonist is IFN-α 2a or IFN-α 2b.

In other embodiments, the invention provides any one of the above-described apparatus in which the interferon receptor agonist is an IFN-β. In other embodiments, the invention provides any one of the above-described apparatus in which the interferon receptor agonist is IFN-tau. In other embodiments, the invention provides any one of the above-described apparatus in which the interferon receptor agonist is IFN-ω.

In other embodiments, the invention provides any one of the above-described apparatus in which the interferon receptor agonist is an IFN-γ.

In another embodiment, the invention provides any of the above-described apparatus in which the sustained pre-selected amount of the interferon receptor agonist in the last sustained dosage interval is at least about 55%, or at least about 60%, or at least about 65%, or at least about 70%, or at least about 75%, or at least about 80%, or at least about 85%, or at least about 90%, or at least about 95%, and up to about 100%, of the maximum tolerated dose (MTD) of the patient.

In another embodiment, the invention provides any of the above-described apparatus in which the sustained pre-selected amount of the interferon receptor agonist in the first sustained dosage interval is at least about 90%, or at least about 100%, and up to about 150%, of the initial pre-selected amount of the interferon receptor agonist, and the sustained pre-selected amount of the interferon receptor agonist in any following sustained dosage interval is at least about 100%, and up to about 150%, of the sustained pre-selected amount of the interferon receptor agonist in the preceding sustained dosage interval. Optionally, the sustained dosage phase consists of only two sustained dosage intervals (a first sustained dosage interval and a single following sustained dosage interval), the sustained pre-selected amount of the interferon receptor agonist in the first sustained dosage interval is about 100% of the initial pre-selected amount of the interferon receptor agonist, the initial dosage phase and the first sustained dosage interval extend for a combined period of time of about 4 weeks, and the sustained pre-selected amount of the interferon receptor agonist in the following sustained dosage interval is about 150% of the sustained pre-selected amount of the interferon receptor agonist in the first sustained dosage interval.

In another embodiment, the invention provides any of the above-described apparatus in which the sustained dosage phase consists of two or three sustained dosage intervals, where the sustained pre-selected amount of the interferon receptor agonist in each following sustained dosage interval is at least about 50% and up to about 70% of the sustained pre-selected amount of the interferon receptor agonist in the preceding sustained dosage interval.

In another embodiment, the invention provides any of the above-described apparatus in which the sustained dosage phase consists of two sustained dosage intervals, and the sustained pre-selected amount of the interferon receptor agonist in the following sustained dosage interval is at least about 50% and up to about 70% of the sustained pre-selected amount of the interferon receptor agonist in the first sustained dosage interval. Optionally, the initial dosage phase and the first sustained dosage interval extend for a combined period of time of about 4 weeks.

In another embodiment, the invention provides any of the above-described apparatus in which the sustained dosage phase consists of three sustained dosage intervals, the sustained pre-selected amount of the interferon receptor agonist in the second sustained dosage interval (the first to occur of the following sustained dosage intervals) is at least about 60% and up to about 70% of the sustained pre-selected amount of the interferon receptor agonist in the first sustained dosage interval, the sustained pre-selected amount of the interferon receptor agonist in the third sustained dosage interval (the last to occur of the following sustained dosage intervals) is about 50% of the sustained pre-selected amount of the interferon receptor agonist in the second sustained dosage interval, the initial dosage phase and the first sustained dosage interval extend for a combined period of time of about 4 weeks, and the second sustained dosage interval extends for a period of time of about 8 weeks.

In another embodiment, the invention provides any of the above-described apparatus in which the interferon receptor agonist is an IFN-α, the sustained pre-selected amount of the IFN-α in the last sustained dosage interval is at least about 0.5 million Units (MU), or at least about 1.0 MU, or at least about 1.5 MU, or at least about 2.0 MU, or at least about 2.5 MU, or at least about 3 MU, or at least about 6 MU, or at least about 9 MU, or at least about 12 MU, or at least about 15 MU, or at least about 18 MU, or at least about 21 MU, or at least about 24 MU, or at least about 27 MU, or at least about 30 MU, and the device is installed to deliver the IFN-α by subcutaneous administration during the initial and sustained dosage phases.

In another embodiment, the invention provides any of the above-described apparatus in which the interferon receptor agonist is an IFN-α, the initial pre-selected amount of the IFN-α is at least about 0.5 million Units (MU), or at least about 1.0 MU, or at least about 1.5 MU, or at least about 2.0 MU, or at least about 2.5 MU, or at least about 3 MU, or at least about 6 MU, or at least about 9 MU, or at least about 12 MU, or at least about 15 MU, or at least about 18 MU, or at least about 21 MU, or at least about 24 MU, or at least about 27 MU, or at least about 30 MU, and the device is installed to deliver the IFN-α by subcutaneous administration during the initial and sustained dosage phases.

In another embodiment, the invention provides any of the above-described apparatus in which the interferon receptor agonist is a consensus interferon, the initial pre-selected amount of the consensus interferon is at least about 0.5 μg, or at least about 1.01 μg, or at least about 1.5 μg, or at least about 2.0 μg, or at least about 2.5 μg, or at least about 3 μg, or at least about 6 μg, or at least about 9 μg, or at least about 12 μg, or at least about 15 μg, or at least about 18 μg, or at least about 21 μg, or at least about 24 μg, or at least about 27 μg, or at least about 30 μg, and the device is installed to deliver the consensus interferon by subcutaneous administration during the initial and sustained dosage phases.

In another embodiment, the invention provides any of the above-described apparatus in which the interferon receptor agonist is a consensus interferon and the sustained pre-selected amount of the consensus interferon in the last sustained dosage interval is at least about 0.5 μg, or at least about 1.0 μg, or at least about 1.5 μg, or at least about 2.0 μg, or at least about 2.5 μg, or at least about 3 μg, or at least about 6 μg, or at least about 9 μg, or at least about 12 μg, or at least about 15 μg, or at least about 18 μg, or at least about 21 μg, or at least about 24 μg, or at least about 27 μg, or at least about 30 μg, and the device is installed to deliver the consensus interferon by subcutaneous administration during the initial and sustained dosage phases.

In another embodiment, the invention provides any of the above-described apparatus in which the sustained pre-selected amount of the interferon receptor agonist in the last sustained dosage interval is at least about 55%, or at least about 60%, or at least about 65%, or at least about 70%, or at least about 75%, or at least about 80%, or at least about 85%, or at least about 90%, or at least about 95%, and up to about 100%, of the maximum tolerated dose (MTD) of the patient.

In another embodiment, the invention provides any of the above-described apparatus in which the initial pre-selected amount of the interferon receptor agonist is at least about 55%, or at least about 60%, or at least about 65%, or at least about 70%, or at least about 75%, or at least about 80%, or at least about 85%, or at least about 90%, or at least about 95%, and up to about 100%, of the maximum tolerated dose (MTD) of the patient.

The invention also features a modification of any of the above-described apparatus in which the set of instructions is altered to incorporate into each period, phase or interval of continuous or substantially continuous delivery of interferon receptor agonist a sleep/wake dosing cycle that is repeated for the duration of any such period, phase or interval, where the sleep/wake cycle delivers the majority of the daily dosage of the interferon receptor agonist to the patient in a substantially continuous or continuous manner during the patient's sleeping hours in any such period, phase or interval. Optionally, the sleep/wake dosing cycle utilizes a pattern of 8 sleeping hours/16 waking hours, or 10 sleeping hours/14 waking hours, or 12 sleeping hours/12 waking hours, for a total of 24 hours in each cycle.

The invention also features a modification of any of the above-described apparatus in which the set of instructions is altered to incorporate into each period, phase or interval of substantially continuous or continuous delivery of interferon receptor agonist a sleep/wake dosing cycle that is repeated for the duration of any such period, phase or interval, where the sleep/wake dosing cycle delivers (i) at least about 50% of the daily dosage of the interferon receptor agonist as a bolus at the beginning or within the first hour of the sleeping hours and (ii) the balance of the daily dosage substantially continuously or continuously during the waking hours for each 24 hour segment in any such period, phase or interval. Optionally, the sleep/wake dosing cycle utilizes a pattern of 8 sleeping hours/16 waking hours, or 10 sleeping hours/14 waking hours, or 12 sleeping hours/12 waking hours, for a total of 24 hours in each cycle.

The invention also features a modification of any of the above-described apparatus in which the set of instructions is altered to incorporate into each period, phase or interval of continuous or substantially continuous delivery of interferon receptor agonist a bolus pulse delivery cycle that is repeated for the duration of any such period, phase or interval, where the bolus pulse cycle provides three or more equal bolus administrations of the interferon receptor, agonist that in the aggregate equal the total dosage of the interferon receptor agonist to be administered to the patient during each 24 hour span of time or fraction thereof in which substantially continuous or continuous delivery of the interferon receptor agonist would otherwise occur, and where the bolus administrations are separated by evenly spaced intervals of time in each bolus pulse delivery cycle. Optionally, the device is an implantable infusion pump and the set of instructions provides for a bolus pulse delivery cycle that utilizes 6 bolus doses administered by the pump at 4 hour intervals in a 24 hour span of time.

The invention also features any of the above-described apparatus in which the set of instructions provides that the duration of the interferon receptor agonist therapy is at least about 24 weeks. Optionally, the duration of the IFN-α therapy is at least about 48 weeks. Optionally, the duration of the interferon receptor agonist therapy is at least about 60 weeks.

In another aspect, the invention features any of the above-described apparatus in which the set of instructions provides that if the user sets the apparatus for the treatment of an antiviral treatment naïve patient having a genotype 1 HCV infection and an initial viral load of greater than 2 million HCV RNA genome copies per ml of serum, then the duration of the interferon receptor agonist therapy is about 48 weeks.

In another aspect, the invention features any of the above-described apparatus in which the set of instructions provides that if the user sets the apparatus for the treatment of an antiviral treatment naïve patient having a genotype 1 HCV infection and an initial viral load of less than or equal to 2 million HCV RNA genome copies per ml of serum, then the duration of the interferon receptor agonist therapy is about 24 weeks up to about 48 weeks.

In another aspect, the invention features any of the above-described apparatus in which the set of instructions provides that if the user sets the apparatus for the treatment of an antiviral treatment naïve patient having a genotype 4 HCV infection, then the duration of the interferon receptor agonist therapy is about 48 weeks.

In another aspect, the invention features any of the above-described apparatus in which the set of instructions provides that if the user sets the apparatus for the treatment of an antiviral treatment naïve patient having a genotype 2 or 3 HCV infection, then the duration of the interferon receptor agonist therapy is about 6 weeks to about 24 weeks.

In another aspect, the invention features any of the above-described apparatus in which the set of instructions provides that if the user sets the apparatus for the treatment of a patient who failed at least one earlier course of antiviral therapy for HCV infection, then the duration of the interferon receptor agonist therapy is about 24 weeks to about 60 weeks.

In another aspect, the invention features any of the above-described apparatus in which the set of instructions provides that if the user sets the apparatus for the treatment of a patient who failed at least one earlier course of IFN-α monotherapy or IFN-α and ribavirin combination therapy for HCV infection, then the duration of the IFN-α therapy performed by the apparatus is about 24 weeks to about 60 weeks. Optionally, the earlier course of IFN-α therapy is either IFN-α 2a or 2b therapy. Optionally, the earlier course of IFN-α therapy is either PEGASYS® peginterferon alfa-2a or PEG-INTRON® peginterferon alfa-2b therapy.

In another aspect, the invention features any of the above-described apparatus in which the interferon receptor agonist is a consensus interferon, and the set of instructions provides that if the user sets the apparatus for the treatment of a patient who failed at least one earlier course of IFN-α monotherapy or IFN-α and ribavirin combination therapy for HCV infection, then the duration of the consensus interferon therapy performed by the apparatus is about 24 weeks to about 60 weeks. Optionally, the earlier course of IFN-α therapy is either IFN-α 2a or 2b therapy. Optionally, the earlier course of IFN-α therapy is either PEGASYS® peginterferon alfa-2a or PEG-INTRON® peginterferon alfa-2b therapy. Optionally, the consensus interferon is INFERGEN® interferon alfacon-1.

In another aspect, the invention features any of the above-described apparatus in which the set of instructions provides that if the user sets the apparatus for the treatment of a patient who has a genotype 2 or 3 HCV infection and who relapsed after responding to at least one earlier course of IFN-α monotherapy or IFN-α and ribavirin combination therapy for HCV infection, then the duration of the interferon receptor agonist therapy performed by the apparatus is about 24 weeks to about 48 weeks. Optionally, the earlier course of IFN-α therapy is either IFN-α 2a or 2b therapy. Optionally, the earlier course of IFN-α therapy is either PEGASYS® peginterferon alfa-2a or PEG-INTRON® peginterferon alfa-2b therapy.

In another aspect, the invention features any of the above-described apparatus in which the interferon receptor agonist is a consensus interferon, and the set of instructions provides that if the user sets the apparatus for the treatment of a patient who has a genotype 2 or 3 HCV infection and who relapsed after responding to at least one earlier course of IFN-monotherapy or IFN-α and ribavirin combination therapy for HCV infection, then the duration of the interferon receptor agonist therapy performed by the apparatus is about 24 weeks to about 48 weeks. Optionally, the earlier course of IFN-α therapy is either IFN-α 2a or 2b therapy. Optionally, the earlier course of IFN-α therapy is either PEGASYS® peginterferon alfa-2a or PEG-INTRON® peginterferon alfa-2b therapy. Optionally, the consensus interferon is INFERGEN® interferon alfacon-1.

In another aspect, the invention features any of the above-described apparatus in which the set of instructions provides that if the user sets the apparatus for the treatment of a patient who has a genotype 1 or 4 HCV infection and who relapsed after responding to at least one earlier course of IFN-α monotherapy or IFN-α and ribavirin combination therapy for HCV infection, then the duration of the interferon receptor agonist therapy is about 48 weeks. Optionally, the earlier course of IFN-α therapy is either IFN-α 2a or 2b therapy. Optionally, the earlier course of IFN-α therapy is either PEGASYS® peginterferon alfa-2a or PEG-INTRON® peginterferon alfa-2b therapy.

In another aspect, the invention features any of the above-described apparatus in which the interferon receptor agonist is a consensus interferon, and the set of instructions provides that if the user sets the apparatus for the treatment of a patient who has a genotype 1 or 4 HCV infection and who relapsed after responding to at least one earlier course of IFN-α monotherapy or IFN-α and ribavirin combination therapy for HCV infection, then the duration of the interferon receptor agonist therapy performed by the apparatus is about 48 weeks. Optionally, the earlier course of IFN-α therapy is either IFN-α 2a or 2b therapy. Optionally, the earlier course of IFN-α therapy is either PEGASYS® peginterferon alfa-2a or PEG-INTRON® peginterferon alfa-2b therapy. Optionally, the consensus interferon is INFERGEN® interferon alfacon-1.

In another aspect, the invention features any of the above-described apparatus in which the set of instructions provides that if the user sets the apparatus for the treatment of a patient who did not respond to at least one earlier course of IFN-α monotherapy or IFN-α and ribavirin combination therapy for HCV infection, then the duration of the interferon receptor agonist therapy performed by the apparatus is about 48 weeks to about 60 weeks. Optionally, the earlier course of IFN-α therapy is either IFN-α 2a or 2b therapy. Optionally, the earlier course of IFN-α therapy is either PEGASYS® peginterferon alfa-2a or PEG-INTRON® peginterferon alfa-2b therapy.

In another aspect, the invention features any of the above-described apparatus in which the interferon receptor agonist is a consensus interferon and the set of instructions provides that if the user sets the apparatus for the treatment of a patient who did not respond to at least one earlier course of IFN-α monotherapy or IFN-α and ribavirin combination therapy for HCV infection, then the duration of the IFN-α therapy performed by the apparatus is about 48 weeks to about 60 weeks. Optionally, the earlier course of IFN-α therapy is either IFN-α 2a or 2b therapy. Optionally, the earlier course of IFN-α therapy is either PEGASYS® peginterferon alfa-2a or PEG-INTRON® peginterferon alfa-2b therapy. Optionally, the consensus interferon is INFERGEN® interferon alfacon-1.

In another aspect, the invention features any of the above-described apparatus in which the set of instructions provides that (a) if the user sets the apparatus for the treatment of an antiviral treatment naïve patient having a genotype 1 HCV infection and an initial viral load of greater than 2 million HCV RNA genome copies/ml of serum, then the duration of the interferon receptor agonist therapy is set at about 48 weeks (b) if the user sets the apparatus for the treatment of an antiviral treatment naïve patient having a genotype 1 HCV infection and an initial viral load of less than or equal to 2 million HCV RNA genome copies/ml of serum, then the duration of the interferon receptor agonist therapy is set at about 24 weeks to about 48 weeks (c) if the user sets the apparatus for the treatment of an antiviral treatment naïve patient having a genotype 2 or 3 HCV infection, then the duration of the interferon receptor agonist therapy is set at about 6 weeks to about 24 weeks (d) if the user sets the apparatus for the treatment of an antiviral treatment naïve patient having a genotype 4 HCV infection, then the duration of the interferon receptor agonist therapy is set at about 48 weeks (e) if the user sets the apparatus for the treatment of a patient who relapsed after responding to an earlier course of IFN-α therapy and who has a genotype 1 or 4 HCV infection, then the duration of the interferon receptor agonist therapy is set at about 48 weeks (f) if the user sets the apparatus for the treatment of a patient who relapsed after responding to an earlier course of IFN-α therapy and who has a genotype 2 or 3 HCV infection, then the duration of the interferon receptor agonist therapy is set at about 24 weeks to about 48 weeks or (g) if the user sets the apparatus for the treatment of a patient who failed to respond to an earlier course of IFN-α therapy, then the duration of the interferon receptor agonist therapy is set at about 48 weeks to about 60 weeks.

In another aspect, the invention features any of the above-described apparatus in which the interferon receptor agonist is an IFN-α, the device provides for the delivery of IFN-α and IFN-γ to the patient, and the set of instructions causes the device to administer to the patient a therapeutically effective amount of IFN-γ for the duration of the IFN-α therapy. In some embodiments, the device contains the IFN-α and IFN-γ in separate drug reservoirs. In other embodiments, the device contains the IFN-α and IFN-γ co-formulated in a single liquid formulation in a single drug reservoir.

In another aspect, the invention features the drug delivery device loaded with IFN-α and IFN-γ in amounts sufficient to administer both drugs to the patient for at least about 1 week, or at least about 2 weeks, or at least about 3 weeks, or at least about 4 weeks, or at least about 1 month, of IFN-α and IFN-γ therapy in connection with the above-described apparatus: In some embodiments, the device contains the IFN-α and IFN-γ in separate drug reservoirs. In other embodiments, the device contains the IFN-α and IFN-γ co-formulated in a single liquid formulation in a single drug reservoir.

In another aspect, the invention provides a drug reservoir or other container containing IFN-α and IFN-γ co-formulated in a liquid in an amount adequate for the administration of both drugs to the patient for at least 1 week, or at least 2 weeks, or at least 3 weeks, or at least 4 weeks, or at least 1 month, using a drug delivery device in connection with the above-described apparatus.

In another aspect, the invention provides a pharmaceutical composition containing IFN-α and IFN-γ in a co-formulated liquid. In some embodiments, the pharmaceutical composition contains an amount of IFN-α and IFN-γ adequate for the administration of both drugs to the patient for at least 1 week, or at least 2 weeks, or at least 3 weeks, or at least 4 weeks, or at least 1 month, using a drug delivery device in connection with the above-described apparatus.

In another aspect, the invention features any of the above-described apparatus in which the interferon receptor agonist is an unPEGylated IFN-α. Optionally, the unPEGylated IFN-α is an unPEGylated consensus interferon.

### BRIEF DESCRIPTION OF THE DRAWING

FIG. 1 depicts the amino acid sequence of IFN-con₁ (the active ingredient of INFERGEN® interferon alfacon-1) (SEQ ID NO. 1).

### DEFINITIONS

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease (as in liver fibrosis that can result in the context of chronic HCV infection); (b) inhibiting the disease, i.e., arresting its development; and (c) relieving the disease, i.e., causing regression of the disease.

The terms “individual,” “host,” “subject,” and “patient” are used interchangeably herein, and refer to a mammal, including, but not limited to, primates, including simians and humans.

The term “dosing event” as used herein refers to administration of an antiviral agent to a patient in need thereof, which event may encompass one or more releases of an antiviral agent from a drug dispensing device. Thus, the term “dosing event,” as used herein, includes, but is not limited to, installation of a continuous delivery device (e.g., a pump or other controlled infusion system); and a single subcutaneous injection followed by infusion controlled by a continuous delivery system.

The term “therapeutically effective amount” is defined as an amount of a therapeutic agent, or a rate of delivery of a therapeutic agent, effective to facilitate a desired therapeutic effect or goal for the treatment of a disease condition. The precise desired therapeutic effect will vary according to the disease condition to be treated, the formulation to be administered, and a variety of other factors that are appreciated by those of ordinary skill in the art.

As used herein, the term “interferon receptor agonist” refers to any agent that binds to an interferon receptor, which binding results in signal transduction via the receptor. Interferon receptor agonists include interferons, including naturally-occurring interferons, modified interferons, synthetic interferons, pegylated interferons, fusion proteins comprising an interferon and a heterologous protein, shuffled interferons; antibody agonists specific for an interferon receptor; chemical agonists; and the like.

The term “Units” refers to units of measurement for quantitation of the ability of the interferon to inhibit the cytopathic effect of a: suitable virus (e.g. encephalomyocarditis virus (EMC), vesicular stomatitis virus, Semliki forest virus) after infection of an appropriate cell line (e.g., the human lung carcinoma cell lines, A549; HEP2/C; and the like). The antiviral activity is normalized to “Units” of antiviral activity exhibited by a reference standard such as human interferon alpha supplied by WHO. Such methods are detailed in numerous references. A particular method for measuring International Units is described in Familletti, P. C., Rubinstein, S and Pestka, S. (1981) “A convenient and rapid cytopathic effect inhibition assay for interferon”, Methods in Enzymol, Vol 78 (S. Pestka, ed), Academic Press, New York pages 387-394. For the most part, the reference standard is human interferon alpha supplied by the World Health Organization, and the method for measuring International Units is that described in Familletti, supra.

The amounts of interferon administered will depend on the specific activities of the compounds and their biological performance in vivo. For example, IFN-α 2b is administered at 11.54 μg protein three times a week corresponding to 3×10⁶ IU per injection (specific activity, 2.68×10⁶ IU/mg). On the other hand, CIFN alfa-con 1 is administered at 9 μg doses per injection corresponding to 9×10⁶ IU per administration (specific activity, 1×10⁹ IU/mg).

The “unPEGylated” or “unpegylated” form(s) of an interferon receptor agonist refers to the subject interferon receptor agonist molecule(s) free of any derivatization with poly (ethylene glycol) (PEG) or other non-proteinaceous polymer moiety, where such derivatization reduces the serum clearance of the derivatized interferon receptor agonist by at least two-fold compared to the serum clearance of the underivatized interferon receptor agonist.

“Continuous delivery” as used herein (e.g., in the context of “continuous delivery of a substance to myocardial tissue”) is meant to refer to movement of drug to a delivery site, e.g., into a tissue in a fashion that provides for delivery of a desired amount of substance into the tissue over a selected period of time, where about the same quantity of drug is received by the patient each minute during the selected period of time.

“Controlled release” as used herein (e.g., in the context of “controlled drug release”) is meant to encompass release of substance (e.g., an interferon receptor agonist, e.g., IFN-α) at a selected or otherwise controllable rate, interval, and/or amount, which is not substantially influenced by the environment of use. “Controlled release” thus encompasses, but is not necessarily limited to, substantially continuous delivery, and patterned delivery (e.g., intermittent delivery over a period of time that is interrupted by regular or irregular time intervals).

“Patterned” or “temporal” as used in the context of drug delivery is meant delivery of drug in a pattern, generally a substantially regular pattern, over a pre-selected period of time (e.g., other than a period associated with, for example a bolus injection). “Patterned” or “temporal” drug delivery is meant to encompass delivery of drug at an increasing, decreasing, substantially constant, or pulsatile, rate or range of rates (e.g., amount of drug per unit time, or volume of drug formulation for a unit time), and further encompasses delivery that is continuous or substantially continuous, or chronic.

The term “controlled drug delivery device” is meant to encompass any device wherein the release (e.g., rate, timing of release) of a drug or other desired substance contained therein is controlled by or determined by the device itself and not substantially influenced by the environment of use, or releasing at a rate that is reproducible within the environment of use.

By “substantially continuous” as used in, for example, the context of “substantially continuous infusion” or “substantially continuous delivery,” it is meant to refer to delivery of drug in a manner that is substantially uninterrupted for a pre-selected period of drug delivery, where the quantity of drug received by the patient during any 8 hour interval in the pre-selected period never falls to zero. Furthermore, “substantially continuous” drug delivery can also encompass delivery of drug at a substantially constant, pre-selected rate or range of rates (e.g., amount of drug per unit time, or volume of drug formulation for a unit time) that is substantially uninterrupted for a pre-selected period of drug delivery.

By “substantially steady state” as used in the context of a biological parameter that may vary as a function of time, it is meant that the biological parameter exhibits a substantially constant value over a time course, such that the area under the curve defined by the value of the biological parameter as a function of time for any 8 hour period during the time course (AUC₈ₕᵣ) is no more than about 20% above or about 20% below, and preferably no more than about 15% above or about 15% below, and more preferably no more than about 10% above or about 10% below, the average area under the curve of the biological parameter over an 8 hour period during the time course (AUC_{8hr average}). The AUC_{8hr average} is defined as the quotient (q) of the area under the curve of the biological parameter over the entirety of the time course (AUCₜₒₜₐₗ) divided by the number of 8 hour intervals in the time course (t_{total1/3days}), i.e., q=(AUCₜₒₜₐₗ)/(t_{total1/3days}). For example, in the context of a serum concentration of a drug, the serum concentration of the drug is maintained at a substantially steady state during a time course when the area under the curve of serum concentration of the drug over time for any 8 hour period during the time course (AUC₈ₕᵣ) is no more than about 20% above or about 20% below the average area under the curve of serum concentration of the drug over an 8 hour period in the time course (AUC_{8hr average}), i.e., the AUC₈ₕᵣ is no more than 20% above or 20% below the AUC_{8hr average} for the serum concentration of the drug over the time course.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “an IFN-α polypeptide” includes a plurality of such polypeptide and reference to “the dosing event” includes reference to one or more dosing events and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently conformed.

### DETAILED DESCRIPTION OF THE INVENTION

The present invention provides methods of treating hepatitis virus infection. The methods generally involve administering an interferon receptor agonist by substantially continuous or continuous delivery. Substantially continuous or continuous delivery of interferon receptor agonist provides for a serum profile of interferon receptor agonist such that a sustained viral response is achieved. Substantially continuous or continuous delivery of an interferon receptor agonist is advantageous, compared to currently available interferon receptor agonist therapies, as discussed below.

Currently available IFN-α therapies for treating HCV infection generally involve subcutaneous injections of IFN-α three times a week (TIW). The kinetics of HCV infection among responders in response to conventional IFN-α therapies, as determined by RNA PCR, have been analyzed. Such studies have clearly shown a rapid viral decline phase in 24-48 hours after the beginning of treatment, resulting in an approximately 0.5-log to an approximately 3-log or greater decrease in serum RNA levels. This early viral response (EVR) is important in reducing the production of viral particles. An early, robust response is generally predictive of a more durable response. This early phase is usually followed by a slower, sustained clearance of the virus over several days or weeks. Generally, this second phase is dependent on characteristics associated with the patient. Without wishing to be bound by any one theory, the second phase reduction in viral titer may be related to removal of virus-infected cells, e.g., by immune system mediated mechanisms. The slope of this second phase is determinative of the sustained viral response (SVR) of the patient, e.g., a steeper second phase slope is generally associated with a SVR and a positive treatment outcome.

Current therapies to treat HCV infection suffer from certain drawbacks. Dosing regimens involving thrice weekly (TIW) injections of IFN-α over extended treatment periods suffer from one or more of the following drawbacks: (1) the dosing regimens are uncomfortable to the patient and, in some cases, result in reduced patient compliance; (2) the dosing regimens are often associated with adverse effects, causing additional discomfort to the patient, and, in some cases, resulting in reduced patient compliance; (3) the dosing regimens result in “peaks” (Cmax) and “troughs” (Cmin) in serum IFN-α concentration, and, during the “trough” periods, virus can replicate, and/or infect additional cells, and/or mutate; (4) in many cases, the log reduction in viral titer during the early viral response is insufficient to effect a sustained viral response that ultimately results in clearance of the virus.

The instant invention provides a delivery profile that avoids these drawbacks, and provides significant advantages, including the following: (1) because the administration is substantially continuous or continuous over the course of treatment, the patient is not subjected to substantial perturbations in drug serum concentration over time, which increases the maximum tolerated dose (MTD) of the patient while reducing patient discomfort and allowing the use of higher dosages than those tolerated by the patient under current dosing regimens; (2) because the dosing is substantially continuous or continuous over the course of treatment, “peaks” (i.e., Cmax) and “troughs” (i.e., Cmin) in serum interferon receptor agonist concentrations are avoided, e.g., the Cmax to Cmin ratio is reduced; (3) because the peak/trough cycles associated with previous dosing regimens are avoided, adverse effects are reduced; (4) because the peak/trough cycles associated with previous dosing regimens are avoided, viral replication, infection of further cells, and mutation is reduced (i.e., there is constant and greater “pressure” on the virus, as there is a more constant and higher level of antiviral agent in the serum); (5) one dosing event according to the invention addresses both the early viral response and the sustained viral responses phases of viral kinetics; (6) the substantially continuous or continuous delivery regimen according to the invention has an effect on the sustained viral response, reducing viral titer still further, and exert enormous negative selective pressure on the virus, reducing viral mutation and/or replication and/or evasion events between dosing cycles); (7) the log reduction in viral titer during substantially continuous or continuous delivery according to the invention is greater than with previously available dosing regimens discussed above; (8) the constant high drug concentration in the sustained phase (Csus) makes the second phase slope steeper; and (9) because the log reduction in viral titer is increased, the outcome during the second phase is more favorable, i.e., the decrease in the viral titer during the sustained viral response phase is more rapid (the slope is steeper) than with previous dosing regimens discussed above.

In some embodiments, the interferon receptor agonist is a Type I interferon receptor agonist. In other embodiments, the interferon receptor agonist is a Type II interferon receptor agonist. In other embodiments, the interferon receptor agonist is a Type III interferon receptor agonist.

### Continuous Delivery of an Interferon Receptor Agonist

Substantially continuous or continuous delivery of an interferon receptor agonist according to the invention provides for a serum concentration of interferon receptor agonist that is in a therapeutically effective window, e.g., the interferon receptor agonist is delivered in such an amount and for such a period of time to provide for an effective amount of interferon receptor agonist in the serum of the individual.

In other embodiments, substantially continuous or continuous delivery of an interferon receptor agonist provides for a relatively constant level of the serum interferon receptor agonist. In some of these embodiments, a bolus dose of interferon receptor agonist is administered, followed by substantially continuous or continuous delivery of a relatively constant amount of interferon receptor agonist. In some of these embodiments, the bolus delivery (e.g., by injection) and the continuous delivery provide for a level of interferon receptor agonist in the serum that is at least about 50%, or at least about 55%, or at least about 60%, or at least about 65%, or at least about 70%, or at least about 75%, or at least about 80%, or at least about 85%, or at least about 90%, or at least about 95%, or about 100%, of the MTD.

In some embodiments, a “therapeutically effective amount” of interferon receptor agonist is an amount that is effective to achieve a 1.5-log, a 2-log, a 2.5-log, a 3-log, a 3.5-log, a 4-log, a 4.5-log, or a 5-log reduction in viral titer in the serum of the individual within a time period of from about 12 hours to about 48 hours, from about 48 hours to about 3 days, from about 3 days to about 7 days, from about 7 days to about 2 weeks, from about 2 weeks to about 4 weeks, from about 4 weeks to about 8 weeks, from about 8 weeks to about 12 weeks, from about 12 weeks to about 16 weeks, from about 16 weeks to about 20 weeks, from about 20 weeks to about 24 weeks, from about 24 weeks to about 48 weeks, or from about 48 weeks to about 60 weeks, after the beginning of the dosing regimen.

Patients with chronic hepatitis C generally have circulating virus at levels of 10⁵-10⁷ genome copies/ml. A therapeutically effective amount of an interferon receptor agonist is an amount that is effective to reduce HCV titer down to about 5×10⁴ to about 10⁵, to about 10⁴ to about 5×10⁴, or to about 5×10³ to about 10⁴ genome copies per milliliter serum.

In some embodiments, an therapeutically effective amount of an interferon receptor agonist is an amount that is effective to reduce HCV titer down to about 5×10⁴ to about 10⁵, to about 10⁴ to about 5×10⁴, or to about 5×10³ to about 10⁴ genome copies per milliliter serum within a period of from about 12 hours to about 48 hours, or from about 16 hours to about 24 hours after the beginning of the dosing regimen.

In some embodiments, a therapeutically effective amount of an interferon receptor agonist for use in the methods of the invention is an amount that is effective to reduce viral titers to undetectable levels, e.g., to about 1000 to about 5000, to about 500 to about 1000, or to about 100 to about 500 HCV RNA genome copies/mL serum. In some embodiments, a therapeutically effective amount of an interferon receptor agonist is an amount that is effective to reduce viral load to lower than 100 HCV RNA genome copies/mL serum.

In some embodiments, a therapeutically effective amount of an interferon receptor agonist for use in the methods of the invention is an amount that is effective to achieve a sustained viral response, e.g., no detectable HCV RNA (e.g., less than about 500, less than about 400, less than about 200, or less than about 100 genome copies per milliliter serum) is found in the patient's serum for a period of at least about one month, at least about two months, at least about three months, at least about four months, at least about five months, or at least about six months following cessation of therapy.

The continuous delivery method of the invention provides for a serum concentration of interferon receptor agonist that is within a therapeutically effective window. The therapeutically effective serum concentration of interferon receptor agonist is maintained for a period of from about 24 hours to about 48 hours, from about 2 days to about 4 days, from about 4 days to about 7 days, from about 1 week to about 2 weeks, from about 2 weeks to about 4 weeks, from about 4 weeks to about 6 weeks, from about 6 weeks to about 8 weeks, from about 8 weeks to about 12 weeks, from about 12 weeks to about 16 weeks, from about 16 weeks to about 20 weeks, from about 20 weeks to about 24 weeks, from about 24 weeks to about 48 weeks, or from about 48 weeks to about 60 weeks.

In some embodiments, the continuous delivery method of the invention provides for a serum concentration of interferon receptor agonist that is at or near the maximum level that is tolerable by the patient for a selected period of time. The serum concentration that is achieved is in a range of from about 10 to about 1000, from about 10 to about 500, from about 20 to about 250, from about 30 to about 100, or from about 50 to about 75 International Units (IU)/ml. The serum concentration is maintained for a period of from about 24 hours to about 48 hours, from about 2 days to about 4 days, from about 4 days to about 7 days, from about 1 week to about 2 weeks, from about 2 weeks to about 4 weeks, from about 4 weeks to about 6 weeks, from about 6 weeks to about 8 weeks, from about 8 weeks to about 12 weeks, from about 12 weeks to about 16 weeks, from about 16 weeks to about 20 weeks, from about 20 weeks to about 24 weeks, from about 24 weeks to about 48 weeks, or from about 48 weeks to about 60 weeks.

In some embodiments, interferon receptor agonist is administered in an amount that is effective to achieve and maintain a serum concentration of interferon receptor agonist that is from about 65% to about 70%, from about 70% to about 75%, from about 75% to about 80%, from about 80% to about 85%, from about 85% to about 90%, from about 90% to about 95%, or from about 95% to about 100% of the maximum tolerated dose (MTD). Thus, within a period of from about 6 hours to about 12 hours, from about 12 hours to about 24 hours, or from about 24 hours to about 48 hours from the beginning of the dosing regimen, a serum concentration interferon-receptor agonist is achieved that is from about 65% to about 70%, from about 70% to about 75%, from about 75% to about 80%, from about 80% to about 85%, from about 85% to about 90%, from about 90% to about 95%, or from about 95% to about 100% of the maximum tolerated dose (MTD). The achieved serum concentration can be maintained for a period of about 7 days to about 2 weeks, from about 2 weeks to about 4 weeks, from about 4 weeks to about 6 weeks, from about 6 weeks to about 8 weeks, from about 8 weeks to about 12 weeks, from about 12 weeks to about 16 weeks, from about 16 weeks to about 20 weeks, from about 20 weeks to about 24 weeks, from about 24 weeks to about 48 weeks, or from about 48 weeks to about 60 weeks.

The total administered daily dose of a consensus interferon for use herein can be about 0.5 μg, about 1.0 μg, about 1.5 μg, about 2.0 μg, about 2.5 μg, about 3 μg, about 9 μg, about 15 μg, about 18 μg, about 21 μg, or about 27 μg/day. Generally, for substantially continuous or continuous administration of a consensus interferon, the methods or devices of the invention employ a delivery rate of from about 0.01 μg/hr, 20 ng/hr, 50 ng/hr or 0.1 μg/hr, 0.25 μg/hr, 1 μg/hr, or up to about 10 μg/hr.

Volume rates are generally from about 0.01 μl/day to about 100 μl/day (i.e., from about 0.0004 μl/hr to about 4 μl/hr), preferably from about 0.04 μl/day to about 10 μl/day, from about 0.2 μl/day to about 5 μl/day, or from about 0.5 μl/day to about 1 μl/day. In some embodiments, the volume/time delivery rate is substantially constant (e.g., delivery is generally maintained at a rate that varies by no more than about 5% to 10% of the cited volume over the cited time period).

In one aspect, the method of the invention provides for treating a patient having an HCV infection by administering to the patient a therapeutically effective amount of an interferon receptor agonist in an initial dosage phase followed by a sustained dosage phase consisting of at least one sustained dosage interval, where during the initial dosage phase an initial serum concentration of the interferon receptor agonist is achieved within a period of time of about 12 hours to about 48 hours, or about 24 hours, after the initial administration of interferon receptor agonist to the patient, where during the first sustained dosage interval a first sustained serum concentration of the interferon receptor agonist of least about 80% and up to about 200% of the initial serum concentration of the interferon receptor agonist is achieved and maintained at a substantially steady state for a period of time of at least about 5 days, and for any following sustained dosage interval a following sustained serum concentration of the interferon receptor agonist of at least about 20% of the first sustained serum concentration of the interferon receptor agonist and at least about 50% and up to about 200% of the sustained serum concentration of the interferon receptor agonist in the preceding sustained dosage interval is achieved and maintained for a period of time of at least about 5 days, and where the duration of the interferon receptor agonist therapy is at least about 6 weeks, or at least about 12 weeks, or at least about 24 weeks, or at least about 48 weeks, or at least about 60 weeks.

In one embodiment, where the interferon receptor agonist is an IFN-α, the IFN-α is administered to the patient in an amount effective to (i) achieve an initial serum concentration of the IFN-α of from about 10 to about 1000, from about 10 to about 500, from about 20 to about 250, from about 30 to about 100, or from about 50 to about 75, Units (U)/ml in the initial dosage phase and (ii) achieve and maintain a sustained serum concentration of the IFN-α in each sustained dosage interval that is at least about 90% and up to about 100% of the initial serum concentration of the IFN-α. Optionally, the sustained dosage phase consists of only one sustained dosage interval.

In another embodiment, where the interferon receptor agonist is an IFN-α, the method of the invention provides for a sustained serum concentration of the IFN-α of from about 10 to about 1000, from about 10 to about 500, from about 20 to about 250, from about 30 to about 100, or from about 50 to about 75, Units (U)/ml that is achieved and maintained in the last sustained dosage interval. Optionally, the sustained dosage phase consists of a single sustained dosage interval.

In another embodiment, interferon receptor agonist is administered to the patient in an amount effective to (i) achieve an initial serum concentration of interferon receptor agonist that is from about 55% to about 60%, from about 65% to about 70%, from about 70% to about 75%, from about 75% to about 80%, from about 80% to about 85%, from about 85% to about 90%, from about 90% to about 95%, or from about 95% to about 100%, of the maximum tolerated dose (ID) of the patient in the initial dosage phase and (ii) achieve and maintain a sustained serum concentration of interferon receptor agonist in each sustained dosage interval that is at least about 90% and up to about 100% of the initial serum concentration. Optionally, the sustained dosage phase consists of a single sustained dosage interval.

In another embodiment, the method of the invention provides for a sustained serum concentration of the interferon receptor agonist in the last sustained dosage interval that is from about 55% to about 60%, from about 65% to about 70%, from about 70% to about 75%, from about 75% to about 80%, from about 80% to about 85%, from about 85% to about 90%, from about 90% to about 95%, or from about 95% to about 100%, of the maximum tolerated dose (MTD) of the patient. Optionally, the sustained dosage phase consists of a single sustained dosage interval.

The initial serum concentration can be achieved by delivery of one or more bolus doses of the interferon receptor agonist, by substantially continuous or continuous delivery of the interferon receptor agonist, or by a combination of a bolus and substantially continuous or continuous delivery. In one embodiment, a continuous delivery device is installed on the patient and used to deliver one or more bolus doses of the interferon receptor agonist to achieve the initial serum concentration of the interferon receptor agonist in the initial dosage phase, and then the installed device is used to provide the substantially continuous or continuous delivery of the interferon receptor agonist during the sustained dosage phase.

In another embodiment, one or more bolus doses of the interferon receptor agonist is delivered by injection to achieve the initial serum concentration of interferon receptor agonist during the initial dosage phase, and then a continuous delivery device is installed on the patient and used to provide substantially continuous or continuous delivery of the interferon receptor agonist during the sustained dosage phase.

In another embodiment, a continuous delivery device is installed on the patient and is used to provide substantially continuous or continuous delivery of the interferon receptor agonist during the initial dosage phase, and one or more bolus doses of the interferon receptor agonist is also administered during the initial dosage phase, where the substantially continuous or continuous delivery of interferon receptor agonist and the bolus dose(s) of interferon receptor agonist are titered to achieve the initial serum concentration of interferon receptor agonist during the initial dosage phase, and then the device is used to provide substantially continuous or continuous delivery of the interferon receptor agonist during the sustained dosage phase.

In another embodiment, a continuous delivery device is installed on the patient and is used to provide substantially continuous or continuous delivery of the interferon receptor agonist (i) to achieve the initial serum concentration of the interferon receptor agonist during the initial dosage phase and (ii) to achieve and maintain the sustained serum concentration of the interferon receptor agonist at a substantially steady state in each sustained dosage interval in the sustained dosage phase.

It will be appreciated that the invention is not limited by the manner of delivery of interferon receptor agonist (e.g., bolus dosage, substantially continuous or continuous delivery, some combination of the foregoing, and the like) and that the invention encompasses the administration of interferon receptor agonist to the patient in any manner that (i) achieves the initial serum concentration of interferon receptor agonist during the initial dosage phase and (i) achieves and maintains the sustained concentration of interferon receptor agonist at a substantially steady state in each sustained dosage interval during the sustained dosage phase, as provided by the methods of the invention.

It will also be understood that the invention does not require a distinction between the interferon receptor agonist regimen employed in the initial dosage phase and the interferon receptor agonist regimen employed in the sustained dosage phase. The invention encompasses any method in which a therapeutically effective serum concentration of the interferon receptor agonist is achieved and maintained at a substantially steady state for the duration of the interferon receptor agonist therapy, i.e., the initial serum concentration of the interferon receptor agonist and each sustained serum concentration of the interferon receptor agonist are substantially the same and are achieved and maintained at a substantially steady state in substantially the same manner throughout the initial and sustained dosage phases.

The invention also encompasses any method in which a therapeutically effective serum concentration of the interferon receptor agonist is achieved and maintained at a substantially steady state during the initial dosage phase and the first sustained dosage interval of the sustained dosage phase, and then an escalated serum concentration of the interferon receptor agonist (i.e., greater than the serum concentration of the interferon receptor agonist in the preceding interval or phase) is achieved and maintained at a substantially steady state in at least one following sustained dosage interval in the sustained dosage phase, i.e., the initial serum concentration of the interferon receptor agonist and the first sustained serum concentration of the interferon receptor agonist are substantially the same and are achieved and maintained at a substantially steady state in substantially the same manner throughout the initial dosage phase and the first sustained dosage interval, and then in at least one following sustained dosage interval a sustained serum concentration of the interferon receptor agonist is employed that reflects an escalation of the interferon receptor agonist dosage.

The invention also encompasses any method in which a therapeutically effective serum concentration of the interferon receptor agonist for induction or loading is achieved and maintained at a substantially steady state during the initial dosage phase and the first sustained dosage interval of the sustained dosage phase, and then a reduced serum concentration of the interferon receptor agonist (below the loading or induction serum concentration of the interferon receptor agonist in the preceding phase or interval) is achieved and maintained at a substantially steady state in at least one following sustained dosage interval, i.e., the initial serum concentration of the interferon receptor agonist and the first sustained serum concentration of the interferon receptor agonist are substantially the same and are achieved and maintained at a substantially steady state in substantially the same manner throughout the initial dosage phase and the first sustained dosage interval, and then in at least one following sustained dosage interval a sustained serum concentration of the interferon receptor agonist is employed that reflects a tapering of the induction or loading interferon receptor agonist dosage.

In many embodiments, the method of the invention employs an implantable infusion pump to provide substantially continuous or continuous delivery of interferon receptor agonist, and optionally bolus delivery of interferon receptor agonist, to the patient. In certain embodiments, the pump is installed to deliver interferon receptor agonist by subcutaneous infusion.

In another aspect, the method of the invention provides for treating a patient having an HCV infection by administering to the patient a therapeutically effective amount of an interferon receptor agonist in an initial dosage phase followed by a sustained dosage phase consisting of at least one sustained dosage interval, where the initial dosage phase extends for a window of time of about 12 hours to about 48 hours and during the initial dosage phase an initial pre-selected amount of the interferon receptor agonist is administered to the patient by a selected route of administration, where during the first sustained dosage interval a first sustained pre-selected amount of the interferon receptor agonist is administered to the patient by the selected route of administration each day in a substantially continuous manner, where the first sustained pre-selected amount of the interferon receptor agonist is at least about 80% and up to about 200% of the initial pre-selected amount of the interferon receptor agonist, and during any following sustained dosage interval a following sustained pre-selected amount of the interferon receptor agonist is administered to the patient by the selected route of administration each day in a substantially continuous manner, where the following sustained pre-selected amount of the interferon receptor agonist is at least about 20% of the first sustained pre-selected amount of the interferon receptor agonist and at least about 50% and up to about 200% of the sustained pre-selected amount of the interferon receptor agonist in the preceding sustained dosage interval, where each sustained dosage interval extends for a period of time of at least about 5 days, and where the duration of the interferon receptor agonist therapy is at least about 6 weeks, or at least about 12 weeks, or at least about 24 weeks, or at least about 48 weeks, or at least about 60 weeks.

Although the rates and/or patterns of interferon receptor agonist delivery used in the initial dosage phase and the sustained dosage phase need not be the same, a common route of administration should be used in the initial and sustained dosage phases. For example, subcutaneous administration of interferon receptor agonist in the initial dosage phase is paired with subcutaneous administration of interferon receptor agonist in the sustained dosage phase.

In one embodiment, the sustained pre-selected amount of interferon receptor agonist administered per day to the patient during the last sustained dosage interval is a dose that is from about 55% to about 60%, from about 65% to about 70%, from about 70% to about 75%, from about 75% to about 80%, from about 80% to about 85%, from about 85% to about 90%, from about 90% to about 95%, or from about 95% to about 100%, of the maximum tolerated dose (TD) of the patient Optionally, the sustained dosage phase consists of a single sustained dosage interval.

The initial pre-selected amount of interferon receptor agonist administered during the initial dosage phase can be delivered by a bolus dose or doses of interferon receptor agonist, by substantially continuous or continuous delivery of the interferon receptor agonist, or by a combination of bolus and substantially continuous or continuous delivery. In one embodiment, a continuous delivery device is installed on the patient and used to deliver the initial pre-selected amount of interferon receptor agonist in one or more bolus doses during the initial dosage phase, and then the installed device is used to deliver the sustained pre-selected amount of the interferon receptor agonist per day by substantially continuous or continuous delivery for each sustained dosage interval. Optionally, the sustained dosage phase consists of a single sustained dosage interval, the initial dosage phase extends for a period of time of about 24 hours, the initial pre-selected amount of the interferon: receptor agonist is delivered to the patient as a single bolus dose by subcutaneous administration at the beginning of the initial dosage phase, and the sustained pre-selected amount of the interferon receptor agonist in the sustained dosage interval is substantially the same as the initial pre-selected amount of the interferon receptor agonist. Optionally, an implantable infusion pump is used to deliver the initial pre-selected amount of the interferon receptor agonist as a bolus and deliver the sustained pre-selected amount of the interferon receptor agonist by substantially continuous or continuous infusion each day in the sustained dosage interval. Optionally, the interferon receptor agonist is an consensus interferon (CIFN), and the initial pre-selected amount of the CIFN and the sustained pre-selected amount of the CIFN are the same and selected from the group consisting of 0.5 μg, 1.0 μg, 1.5 μg, 2.0 μg, 2.5 μg, 3 μg, 6 μg, 9 μg, 15 μg, 18 μg, 21 μg, 24 μg, 27 μg, and 30 μg of the consensus interferon (CIFN). Optionally, the CIFN is INFERGEN® interferon alfacon-1.

In another embodiment, the initial pre-selected amount of the interferon receptor agonist is delivered in one or more bolus doses by injection during the initial dosage phase, and then a delivery device is installed on the patient and used to deliver the sustained pre-selected amount of the interferon receptor agonist per day by substantially continuous or continuous infusion for each sustained dosage interval.

In another embodiment, a delivery device is installed on the patient and is used to (i) provide substantially continuous or continuous delivery of the interferon receptor agonist during the initial dosage phase and (ii) deliver one or more bolus doses of the interferon receptor agonist during the initial dosage phase, where the substantially continuous or continuous delivery of interferon receptor agonist and the bolus dose(s) of interferon receptor agonist are titered to provide an aggregate amount of interferon receptor agonist equal to the initial pre-selected amount of interferon receptor agonist during the initial dosage phase, and then the delivery device is used to deliver the sustained pre-selected amount of the interferon receptor agonist per day by substantially continuous or continuous infusion for each sustained dosage interval.

In another embodiment, a delivery device is installed on the patient and is used to (i) deliver the initial pre-selected amount of interferon receptor agonist by substantially continuous or continuous infusion during the initial dosage phase and (ii) deliver the sustained pre-selected amount of interferon receptor agonist per day by substantially continuous or continuous infusion for each sustained dosage interval.

It will be appreciated that the invention is not limited by the manner of delivery of interferon receptor agonist (e.g., bolus dosage, continuous delivery, some combination of the foregoing, and the like) and that the invention encompasses the administration of interferon receptor agonist to the patient in any manner that (i) delivers the initial pre-selected amount of interferon receptor agonist during the initial dosage phase and (ii) delivers the sustained pre-selected amount of interferon receptor agonist per day in a substantially continuous or continuous manner for each sustained dosage interval, as provided by the methods of the invention.

In many embodiments, the method of the invention employs an implantable infusion pump to provide substantially continuous or continuous delivery of interferon receptor agonist, and optionally bolus delivery of interferon receptor agonist, during the initial and/or sustained dosage phases of the dosing regimens. In certain embodiments, the pump is installed to deliver interferon receptor agonist by subcutaneous infusion.

It will also be understood that the invention does not require any particular distinction, or any distinction at all, between the interferon receptor agonist regimen employed in the initial dosage phase and the interferon receptor agonist regimen employed in the sustained dosage phase. The invention encompasses any method in which a therapeutically effective, pre-selected amount of the interferon receptor agonist is administered to the patient each day by substantially continuous or continuous delivery for the duration of the interferon receptor agonist therapy, i.e., the initial pre-selected amount of the interferon receptor agonist and each sustained pre-selected amount of the interferon receptor agonist are substantially the same and are substantially continuously or continuously delivered each day in substantially the same manner throughout the initial and sustained dosage phases.

The invention also encompasses any method in which a therapeutically effective, pre-selected amount of the interferon receptor agonist is administered each day by substantially continuous or continuous delivery during the initial dosage phase and the first sustained dosage interval, and then an escalated pre-selected amount of the interferon receptor agonist (i.e., greater than the pre-selected amount of the interferon receptor agonist in the preceding interval or phase) is administered each day by substantially continuous or continuous delivery in at least one following sustained dosage interval, i.e., the initial pre-selected amount of the interferon receptor agonist and the first sustained pre-selected amount of the interferon receptor agonist are substantially the same and are substantially continuously or continuously delivered in substantially the same manner throughout the initial dosage phase and the first sustained dosage interval, and then in at least one following sustained dosage interval a sustained pre-selected amount of the interferon receptor agonist is employed that escalates the interferon receptor agonist dosage.

In one embodiment, the sustained dosage phase consists of two sustained dosage intervals, the interferon receptor agonist is a consensus interferon (CIFN), the initial pre-selected amount of the CIFN and the first sustained pre-selected amount of the CIFN are 12 μg CIFN/day, the following sustained pre-selected amount of the IFN-α is 18 μg CIFN/day, and the initial dosage phase and the first sustained dosage interval extend for a combined period of time of about 4 weeks.

The invention also encompasses any method in which a therapeutically effective, pre-selected amount of the interferon receptor agonist for loading or induction dosing is administered each day by substantially continuous or continuous delivery during the initial dosage phase and the first sustained dosage interval, and then a reduced amount of the interferon receptor agonist (below the loading or induction amount) is administered each day by substantially continuous or continuous delivery in at least one following sustained dosage interval, i.e., the initial pre-selected mount of the interferon receptor agonist and the first sustained pre-selected amount of the interferon receptor agonist are the same and are substantially continuously or continuously delivered in substantially the same manner throughout the initial dosage phase and the first sustained dosage interval, and then in at least one following sustained dosage interval a sustained pre-selected amount of the interferon receptor agonist is employed that reflects a tapering of the induction or loading dosage.

In one embodiment, the sustained dosage phase consists of two sustained dosage intervals, the interferon receptor agonist is a consensus interferon (CIFN), the initial pre-selected and the first sustained pre-selected amounts of the CIFN are 15 μg CIFN/day, the sustained pre-selected amount of the CIFN in the following sustained dosage interval is 9 μg CIFN/day, the initial dosage phase and the first sustained dosage interval extend for a combined period of time of about 8 weeks, and the following sustained dosage interval extends for a period of time of about 16 weeks to about 40 weeks.

In another embodiment, the sustained dosage phase consists of two sustained dosage intervals, the interferon receptor agonist is a consensus interferon (CIFN), the initial pre-selected and first sustained pre-selected amounts of the CIFN are 27 μg CIFN/day, the following pre-selected amount of the CIFN is 18 μg CIFN/day, the initial dosage phase and the first sustained dosage interval extend for a combined period of time of about 4 weeks, and the following dosage interval extends for a period of time of about 16 weeks to about 44 weeks.

In another embodiment, the sustained dosage phase consists of three sustained dosage intervals, the interferon receptor agonist is a consensus interferon (CIFN), the initial pre-selected and first sustained preselected amounts of the CIFN are 27 μg CIFN/day, the sustained pre-selected amount of the CIFN in the second sustained dosage interval (the first to occur of the following sustained dosage intervals) is 18 μg CIFN/day, the sustained pre-selected amount of the CIFN in the third sustained dosage interval (the last to occur of the following sustained dosage intervals) is 9 μg CIFN/day, the initial dosage phase and the first sustained dosage interval extend for a combined period of time of about 4 weeks, the second sustained dosage interval extends for a period of time of about 8 weeks, and the third sustained dosage interval extends for a period of time of about 12 weeks to about 36 weeks.

In another embodiment, the sustained dosage phase consists of two sustained dosage intervals, the interferon receptor agonist is a consensus interferon (CIFN), the initial pre-selected and first sustained pre-selected amounts of the CIFN are 18 μg CIFN/day, the sustained pre-selected amount of the CIFN in the following sustained dosage interval is 9 μg CIFN/day, the initial dosage phase and the first sustained dosage interval extend for a combined period of time of about 4 weeks, and the following sustained dosage interval extends for a period of time of about 20 weeks to about 44 weeks.

In addition, the invention provides a modification of any of the methods described above where the subject method is altered to include sleep/wake dosing cycles during at least the sustained dosage phase or treatment period. The sleep/wake dosing cycle is designed to deliver the majority of the daily dosage of interferon receptor agonist to the patient during sleeping hours, thereby reducing the frequency and severity of side effects experienced by the patient in his/her waking hours.

In one example, the method of the invention provides for treating a patient having an HCV infection by administering to the patient a therapeutically effective amount of an interferon receptor agonist for a treatment period of at least about 6 weeks, or at least about 12 weeks, or at least about 24 weeks, or at least about 48 weeks, or at least about 60 weeks, where a sustained serum concentration of the interferon receptor agonist is achieved and maintained at a substantially steady state during the sleeping hours of the patient for the duration of the treatment period.

In another example, the method of the invention provides for treating a patient having an HCV infection by administering to the patient a therapeutically effective amount of an interferon receptor agonist in an initial dosage phase followed by a sustained dosage phase consisting of at least one sustained dosage interval, where during the initial dosage phase an initial serum concentration of the interferon receptor agonist is achieved within a period of time of about 12 hours to about 48 hours, or about 24 hours, after the initial administration of the interferon receptor agonist to the patient, where during the first sustained dosage interval a first sustained serum concentration of the interferon receptor agonist of at least about 80% and up to about 200% of the initial serum concentration of the interferon receptor agonist is achieved and maintained at a substantially steady state during the sleeping hours of the patient and allowed to decay during the waking hours of the patient, and for any following sustained dosage interval a following sustained serum concentration of the interferon receptor agonist of at least about 20% of the first sustained serum concentration of the interferon receptor agonist and at least about 50% and up to about 200% of the sustained serum concentration of the interferon receptor agonist in the preceding sustained dosage interval is achieved and maintained at a substantially steady state during the sleeping hours of the patient and allowed to decay during the waking hours of the patient, where each sustained dosage interval extends for a period of time of at least about 5 days, and where the duration of the interferon receptor agonist therapy is at least about 6 weeks, or at least about 12 weeks, or at least about 24 weeks, or at least about 48 weeks, or at least about 60 weeks.

In another embodiment, the sustained serum concentration of the interferon receptor agonist is achieved and maintained by substantially continuous or continuous delivery of the interferon receptor agonist to the patient during the sleeping hours of the patient in each sustained dosage interval or treatment period.

Ordinarily, the administration of interferon receptor agonist is controlled to accommodate sleep/wake cycles ranging from a cycle of about 8 sleeping hours/16 waking hours to a cycle of about 12 waking hours/12 sleeping hours, or a sleep/wake cycle of about 10 sleeping hours/14 waking hours, per 24 hour period. Of course, the sleep/wake cycle can be tailored to the specific medical needs or individual preferences of the patient.

In some embodiments, the sustained serum concentration of the interferon receptor agonist in each sustained dosage interval or treatment period is achieved and maintained at a substantially steady state during the patient's sleeping hours and a lower serum concentration of the interferon receptor agonist (e.g., lower than the sustained serum concentration and low enough to moderate or avoid side effects) is achieved and maintained at a substantially steady state during the patient's waking hours for the duration of the sustained dosage interval or treatment period. Optionally, the interferon receptor agonist is delivered to the patient in a substantially continuous or continuous manner during each sustained dosage interval or treatment period.

In another example, the method of the invention provides for treating a patient having an HCV infection by administering to the patient a therapeutically effective amount of an interferon receptor agonist for a treatment period of at least about 6 weeks, or at least about 12 weeks, or at least about 24 weeks, or at least about 48 weeks, or at least about 60 weeks, where a sustained pre-selected amount of the interferon receptor agonist is administered to the patient by substantially continuous or continuous delivery each day during the sleeping hours of the patient.

In another example, the method of the invention provides for treating a patient having an HCV infection by administering to the patient a therapeutically effective amount of an interferon receptor agonist in an initial dosage phase followed by a sustained dosage phase consisting of at least one sustained dosage interval, where the initial dosage phase extends for a period of time of about 12 hours to about 48 hours, or about 24 hours, and at the beg of the initial dosage phase an initial pre-selected amount of the interferon receptor agonist is administered to the patient by a selected route of administration, and where during the first sustained dosage interval a first sustained pre-selected amount of the interferon receptor agonist is administered to the patient each day by the selected route of administration in a substantially continuous or continuous manner during the sleeping hours of the patient, where the first sustained pre-selected amount of the interferon receptor agonist is at least about 80% and up to about 200% of the initial pre-selected amount of the interferon receptor agonist, and during any following sustained dosage interval a following sustained pre-selected amount of the interferon receptor agonist is administered to the patient each day by the selected route of administration in a substantially continuous or continuous manner during the sleeping hours of the patient, where the following sustained pre-selected amount of the interferon receptor agonist is at least about 20% of the first sustained pre-selected amount of the interferon receptor agonist and at least about 50% and up to about 200% of the sustained pre-selected amount of the interferon receptor agonist in the preceding sustained dosage interval, where each sustained dosage interval extends for a period of time of at least about 5 days, and where the duration of the interferon receptor agonist therapy is at least about 6 weeks, or at least about 12 weeks, or at least about 24 weeks, or at least about 48 weeks, or at least about 60 weeks.

Ordinarily, the administration of interferon receptor agonist is controlled to accommodate sleep/wake-cycles ranging from a cycle of about 8 sleeping hours/16 waking hours to a cycle of about 12 waking hours/12 sleeping hours, or a sleep/wake cycle of about 10 sleeping hours/14 waking hours, per 24 hour period. Obviously, the clinician can tailor the sleep/wake cycle to the particular medical needs or individual preferences of the patient.

In some embodiments, the delivery of interferon receptor agonist is controlled to deliver the major portion of the sustained pre-selected amount of the interferon receptor agonist during the patient's sleeping hours and to deliver the remainder of the sustained pre-selected amount of the interferon receptor agonist during the patient's waking hours for each 24 hour time span in each sustained dosage interval or the treatment period, where the remainder is made small enough to moderate or avoid side effects during the patient's waking hours. In one embodiment in which an implantable infusion pump is used to provide substantially continuous or continuous delivery of the interferon receptor agonist, the remainder is limited to a negligible portion of the sustained pre-selected amount of the interferon receptor agonist for each sustained dosage interval, but is nevertheless sufficient to maintain the drug lubrication of components in the pump during the waking hours in the sustained dosage interval or treatment period.

In another aspect, the invention provides a modification of any of the methods described above where the subject method is altered to incorporate a sleep/wake cycle in at least the sustained dosage phase or treatment period in which at least about 50% of the daily dosage of interferon receptor agonist is delivered as a bolus at the beginning or within the first hour of the sleeping hours and the balance of the daily dosage is delivered substantially continuously or continuously during the waking hours for each 24 hour interval in the sustained dosage phase or treatment period.

In one example, the method of the invention provides for treating a patient having an HCV infection by administering to the patient a therapeutically effective amount of an interferon receptor agonist in an initial dosage phase followed by a sustained dosage phase consisting of at least one sustained dosage interval, where the initial dosage phase extends for a period of about 12 hours to about 48 hours and during the initial dosage phase an initial pre-selected amount of the interferon receptor agonist is administered to the patient by a selected route of administration, where during the first sustained dosage interval a first sustained pre-selected amount of the interferon receptor agonist is administered to the patient each day by the selected route of administration and is at least about 80% and up to about 200% of the initial pre-selected amount of the interferon receptor agonist, where during any following sustained dosage interval a following sustained pre-selected amount of the interferon receptor agonist is administered to the patient each day by the selected route of administration and is at least about 20% of the first sustained pre-selected amount of the interferon receptor agonist and at least about 50% and up to about 200% of the sustained pre-selected amount of the interferon receptor agonist in the preceding sustained dosage interval, where at least 50% of the sustained pre-selected amount of the interferon receptor agonist is delivered as a bolus at the beginning or within the first hour of the sleeping hours and the undelivered remainder of the sustained pre-selected amount is delivered substantially continuously or continuously during the waking hours of the patient for each 24 hour period in each sustained dosage interval, where each sustained dosage interval extends for a period of time of at least about 5 days, and where the duration of the interferon receptor agonist therapy is at least about 6 weeks, or at least about 12 weeks, or at least about 24 weeks, or at least about 48 weeks, or at least about 60 weeks.

In another example, the method of the invention provides for treating a patient having an HCV infection by administering to the patient a therapeutically effective amount of an interferon receptor agonist for a treatment period of at least about 6 weeks, or at least about 12 weeks, or at least about 24 weeks, or at least about 48 weeks, or at least about 60 weeks, where a sustained pre-selected amount of the interferon receptor agonist is administered to the patient each day, where at least about 50% of the sustained pre-selected amount of the interferon receptor agonist is delivered as a bolus at the beginning or within the first hour of the sleeping hours and the undelivered remainder of the sustained pre-selected amount is delivered substantially continuously or continuously during the waking hours of the patient for each 24 hour interval in the treatment period. Optionally, an implantable infusion pump is installed on the patient and used to effect and control the bolus and substantially continuous or continuous delivery of the interferon receptor agonist.

Ordinarily, the delivery of the interferon receptor agonist is controlled to accommodate sleep/wake cycles ranging from a cycle of about 8 sleeping hours/16 waking hours to a cycle of about 12 sleeping hours/12 waking hours, or a sleep/wake cycle of about 10 sleeping hours/14 waking hours per 24 hour interval in the sustained dosage phase or treatment period. Optionally, the interferon receptor agonist is a consensus interferon (CIFN), and the sustained pre-selected amount of the CIFN per 24 hour interval is apportioned in a total:remainder ratio selected from the group consisting of 45 μg:15 μg, 39 μg:12 μg, 30 μg:15 μg, 27 μg:12 μg, 27 μg:6 μg, 24 μg:6 μg, 21 μg:6 μg, 18 μg:6 μg, 15 μg:6 μg, 12 μg:6 μg, 10 μg:5 μg, 8 μg:4 μg and 6 μg:3 μg of a consensus interferon (CIFN). Optionally, the CIFN is INFERGEN® interferon alfacon-1.

It will be appreciated that the sleep/wake aspect of the invention does not require any particular distinction, or any distinction at all, between the interferon receptor agonist regimen employed in the initial dosage phase and the interferon receptor agonist regimen employed in the sustained dosage phase. For example, the initial serum concentration of the interferon receptor agonist and the initial pre-selected amount of the interferon receptor agonist can be substantially the same and implemented with substantially the same pattern of delivery in substantially the same manner as the sustained serum concentration of the interferon receptor agonist and the sustained pre-selected amount of the interferon receptor agonist, respectively, for each sustained dosage interval in the sustained dosage phase.

In another example, a pre-selected amount or serum concentration of the interferon receptor agonist is implemented with a uniform pattern of delivery in a uniform manner in the initial dosage phase and in the first sustained dosage interval of the sustained dosage phase, and then an escalated amount or serum concentration of the interferon receptor agonist (i.e., greater than the amount or serum concentration of the interferon receptor agonist employed in the prior phase or interval) is implemented with the uniform pattern of delivery in the uniform manner in at least one following sustained dosage interval in the sustained dosage phase.

In another example, a loading or induction pre-selected amount or serum concentration of the interferon receptor agonist is implemented with a uniform pattern of delivery in a uniform manner in the initial dosage phase and in the first sustained dosage interval, and then a tapered amount or serum concentration of the interferon receptor agonist (i.e., lower than the loading or induction amount or serum concentration of the interferon receptor agonist in the prior phase or interval) is implemented with the uniform pattern of delivery in the uniform manner in at least one following sustained dosage interval in the sustained dosage phase.

The invention also features a modification of any of the above-described methods in which each period or phase of substantially continuous or continuous administration of interferon receptor agonist to the patient is altered to incorporate a bolus pulse delivery cycle that is repeated for the duration of any such period or phase in the subject method, where the bolus pulse cycle provides three or more equal bolus administrations of the interferon receptor agonist that in the aggregate equal the total dosage of the interferon receptor agonist to be administered to the patient during each 24 hour span of time or fraction(s) thereof in which substantially continuous or continuous delivery of interferon receptor agonist would otherwise occur, and where the bolus administrations are separated by evenly spaced intervals of time in each bolus pulse delivery cycle.

It will be appreciated that the bolus pulse aspect of the invention is not limited by any maximum number of bolus pulse doses or time intervals in the bolus pulse delivery cycle. Instead, the method of the invention can be practiced with any number of bolus doses or time intervals in the bolus pulse delivery cycle that is within the physical capabilities of the selected drug delivery device.

In one embodiment, the invention provides a method of treating a patient having an HCV infection by administering to the patient a therapeutically effective amount of an interferon receptor agonist in an initial dosage phase followed by a sustained dosage phase consisting of at least one sustained dosage interval, where the initial dosage phase extends for a period of time of about 12 hours to about 48 hours and an initial pre-selected amount of the interferon receptor agonist is administered to the patient by a selected route of administration at the beginning of the initial dosage phase, where during the first sustained dosage interval a first sustained pre-selected amount of the interferon receptor agonist is administered to the patient each day by the selected route of administration in a bolus pulse delivery cycle, where the first sustained pre-selected amount of the interferon receptor agonist is at least about 80% and up to about 200% of the initial pre-selected amount of the interferon receptor agonist, and during any following sustained dosage interval a following sustained pre-selected amount of the interferon receptor agonist is administered to the patient each day by the selected route of administration in the bolus pulse delivery cycle and is at least about 20% of the first sustained pre-selected amount of the interferon receptor agonist and at least about 50% and up to about 200% of the sustained pre-selected amount of the interferon receptor agonist in the preceding sustained dosage interval, and for each sustained dosage interval (1) the bolus pulse delivery cycle consists of at least three equal bolus doses of the interferon receptor agonist that (a) in the aggregate equal the sustained pre-selected amount of the interferon receptor agonist in the sustained dosage interval and (b) are administered to the patient by a delivery device at evenly spaced intervals of time in a 24 hour period and (2) the bolus pulse delivery cycle is repeated for the duration of the sustained dosage interval, where each sustained dosage interval extends for a period of time of at least about 5 days, and where the duration of the interferon receptor agonist therapy is at least about 6 weeks, or at least about 12 weeks, or at least about 24 weeks, or at least about 48 weeks, or at least about 60 weeks.

Optionally, the bolus pulse delivery cycle uses six bolus doses where the bolus doses are administered by an implantable infusion pump at 4 hour intervals during each 24 hour period in each sustained dosage interval.

It will be understood that the bolus pulse delivery cycle aspect of the invention does not require any particular distinction, or any distinction at all, between the interferon receptor agonist regimen employed in the initial dosage phase and the interferon receptor agonist regimen employed in the sustained dosage phase. For example, the initial pre-selected amount of the interferon receptor agonist can be substantially the same and implemented with substantially the same pattern of delivery in substantially the same manner as the sustained pre-selected amount of the interferon receptor agonist for each sustained dosage interval in the sustained dosage phase.

In another example, the initial pre-selected amount of the interferon receptor agonist and the first sustained pre-selected amount of the interferon receptor agonist are the same and are implemented with a uniform pattern of delivery in a uniform manner in the initial dosage phase and in the first sustained dosage interval, and then an escalated amount of the interferon receptor agonist (i.e., greater than the amount of the interferon receptor agonist used in the prior phase or interval) is implemented with the uniform pattern of delivery in the uniform manner in at least one following sustained dosage interval in the sustained dosage phase.

In another example, a loading or induction amount of the interferon receptor agonist is implemented with a uniform pattern of delivery in a uniform manner in the initial dosage phase and in the first sustained dosage interval, and then a tapered amount of the interferon receptor agonist (i.e., lower hand the loading or induction amount of the interferon receptor agonist in the preceding phase or interval) is implemented with the uniform pattern of delivery in the uniform manner in at least one following sustained dosage interval in the sustained dosage phase.

In another embodiment, the invention provides a method for treating a patient having an HCV infection by administering to the patient a therapeutically effective amount of an interferon receptor agonist for a treatment period of at least about 6 weeks, or at least about 12 weeks, or at least about 24 weeks, or at least about 48 weeks, or at least about 60 weeks, where a sustained pre-selected amount of the interferon receptor agonist is administered to the patient each day in a bolus pulse delivery cycle, and where the bolus pulse delivery cycle (1) consists of at least three equal bolus doses of the interferon receptor agonist that (a) in the aggregate equal the sustained pre-selected amount of the interferon receptor agonist and (b) are administered to the patient by a delivery device at evenly spaced intervals of time in a 24 hour period and (2) is repeated for the duration of the treatment period. Optionally, the interferon receptor agonist is a consensus interferon (CIFN), the sustained pre-selected amount of the CIFN is about 15 μg, 18 μg, 21 μg, 27 μg, or 30 μg of CIFN and the bolus pulse delivery cycle consists of 6 equal doses of the CIFN administered by an implantable infusion pump at 4 hour intervals of time in a 24 hour period according to the schedules shown in Table 1 below.

TABLE 1
Bolus Pulse Delivery Schedules

| Dose | | | | | | TOTAL |
|---|---|---|---|---|---|---|
| Administered | At | At | At Hour | At Hour | At Hour | DAILY |
| at Hour 1 | Hour 5 | Hour 9 | 13 | 17 | 21 | DOSE |
| 5 μg | 5 μg | 5 μg | 5 μg | 5 μg | 5 μg | 30 μg |
| 4.5 μg | 4.5 μg | 4.5 μg | 4.5 μg | 4.5 μg | 4.5 μg | 27 μg |
| 3.5 μg | 3.5 μg | 3.5 μg | 3.5 μg | 3.5 μg | 3.5 μg | 21 μg |
| 3 μg | 3 μg | 3 μg | 3 μg | 3 μg | 3 μg | 18 μg |
| 2.5 μg | 2.5 μg | 2.5 μg | 2.5 μg | 2.5 μg | 2.5 μg | 15 μg |

As described in more detail below, in any of the above-described methods, the interferon receptor agonist is an agonist of a Type I interferon receptor, an agonist of a Type II interferon receptor, or an agonist of a Type III interferon receptor. In particular embodiments, in any of the above-described methods, the interferon receptor agonist is an IFN-α. In particular embodiments, in any of the above-described methods, the interferon receptor agonist is a CIFN. In particular embodiments, in any of the above-described methods, the interferon receptor agonist is INFERGEN® interferon alfacon-1. In other particular embodiments, in any of the above-described methods, the interferon receptor agonist is IFN-α 2a or IFN-α 2b. In preferred embodiments, where the interferon receptor agonist is an IFN-α, the IFN-α administered to the patient according to the methods of the invention is an unPEGylated IFN-α.

### Drug Delivery Systems

The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present invention. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, the present methods of drug delivery can be accomplished using any of a variety of refillable, pump systems. Pumps provide consistent, controlled release over time. Typically, the agent (e.g., the interferon receptor agonist, e.g., IFN-α, IFN-β, IFN-γ, etc.) is in a liquid formulation in a drug-impermeable reservoir, and is delivered in a continuous fashion to the individual. In a preferred embodiment, the MiniMed—Model 508 continuous infusion pump (manufactured by Medtronics, Inc.) is used.

In some embodiments, e.g., where the device provides for a multiphasic serum interferon receptor agonist profile, the device is programmable, such that for a first pre-selected time period, a first concentration of interferon receptor agonist is delivered, and, for a second pre-selected time period, a second concentration of interferon receptor agonist is delivered.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to a subdermal, subcutaneous, intramuscular, or other suitable site within a subject's body. Subcutaneous implantation sites are generally preferred because of convenience in implantation and removal of the drug delivery device.

Drug release devices suitable for use in the invention may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g. where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present invention. Examples of such devices include those described in, for example, U.S. Pat. Nos. 4,692,147; 4,360,019; 4,487,603; 4,360,019; 4,725,852, and the like. In general, the present methods of drug delivery can be accomplished using any of a variety of refillable, non-exchangeable pump systems. Pumps and other convective systems are generally preferred due to their generally more consistent, controlled release over time. Osmotic pumps are particularly preferred due to their combined advantages of more consistent controlled release and relatively small size (see, e.g., PCT published application no. WO 97/27840 and U.S. Pat. Nos. 5,985,305 and 5,728,396)). Exemplary osmotically-driven devices suitable for use in the invention include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted supra, an implantation site is a site within the body of a subject at which a drug delivery device is introduced and positioned. Implantation sites include, but are not necessarily limited to a subdermal, subcutaneous, intramuscular, or other suitable site within a subject's body.

### Controlled Delivery Apparatus for Delivery of Interferon Receptor Agonist

The invention further provides controlled delivery apparatus designed to effect any of the methods described herein, where the apparatus includes a delivery device and a unit that automatically controls the delivery device to effect the delivery of interferon receptor agonist to the patient according to the subject method. In some embodiments, the control unit is not designed to accept user input. In these embodiments, the system is manufactured with the control unit pre-set to accomplish any of the above-described methods for delivery of interferon receptor agonist using a particular route of administration and a particular drug delivery device.

In other embodiments, the control unit is designed to allow the user to select a desired course of treatment from two or more of the treatment methods described herein for use in connection with a particular route of administration and a particular drug delivery device.

In other embodiments, the control unit is designed to allow the user to (i) select a desired course of treatment from two or more of the treatment methods described herein for use in connection with a particular route of administration and a particular drug delivery device and (ii) select from a fixed set of values one or more of the parameters in the selected course of treatment (e.g., the initial and/or sustained serum concentration of the interferon receptor agonist, the initial dosage phase and/or sustained dosage interval period of time, the duration of the interferon receptor agonist therapy, the pre-selected amount of interferon receptor agonist, the sleep/wake cycle, the bolus pulse delivery cycle, etc.).

In other embodiments, the control unit is designed to allow the user to (i) select a desired course of treatment from two or more of the treatment methods described herein for use in connection with a particular route of administration and a particular drug delivery device and (ii) select within a fixed range of values one or more of the parameters in the selected course of treatment (e.g., the initial and/or sustained serum concentration of the interferon receptor agonist, the initial dosage phase and/or sustained dosage interval period of time, the duration of the interferon receptor agonist therapy, the pre-selected amount of interferon receptor agonist; the sleep/wake cycle, the bolus pulse delivery cycle, etc.).

The apparatus of the invention are designed for use in connection with an appropriate device for delivery of an interferon receptor agonist by a suitable route of administration. Optionally, the apparatus of the invention employs subcutaneous administration of the interferon receptor agonist to the patient. In other embodiments, the apparatus of the invention employ a device that is an implantable infusion pump for delivery of the interferon receptor agonist to the patient by subcutaneous infusion.

It will be understood that programmable and non-programmable embodiments are included in the controlled delivery apparatus of the invention. In the programmable embodiments, the control unit is controlled by a set of instructions that can be altered by the user. In the non-programmable embodiments, the control unit is controlled by a set of instructions that cannot be altered by the user.

In one example, the invention provides an apparatus for the controlled delivery of an interferon receptor agonist to a patient having an HCV infection, where (i) the apparatus includes a device for delivery of the interferon receptor agonist to the patient by a selected route of administration, (ii) the apparatus includes a control unit that is operated by a series of commands, (iii) the series of commands contains a set of instructions that causes the device to administer a therapeutically effective amount of the interferon receptor agonist to the patient via the selected route of administration in a manner effective to achieve an initial dosage phase followed by a sustained dosage phase consisting of at least one sustained dosage interval, where during the initial dosage phase an initial serum concentration of the interferon receptor agonist is achieved within a period of time of about 12 hours to about 48 hours, where during the first sustained dosage interval a first sustained serum concentration of the interferon receptor agonist is achieved and then maintained at a substantially steady state for a period of time of at least about 5 days, where the first sustained serum concentration is at least about 80% and up to about 200% of the initial serum concentration, and during any following sustained dosage interval a following sustained serum concentration of the interferon receptor agonist is achieved and then maintained at a substantially steady state for a period of time of at least about 5 days, where the following sustained serum concentration is at least about 20% of the first sustained serum concentration and at least about 50% and up to about 200% of the sustained serum concentration in the preceding sustained dosage interval, and where the duration of the interferon receptor agonist therapy is at least about 6 weeks, or at least about 12 weeks, or at least about 24 weeks, or at least about 48 weeks, or at least about 60 weeks, and (iv) when the apparatus is in place and operational on the patient, the control unit executes the set of instructions in the series of commands.

This example of the invention includes embodiments where the initial and/or sustained serum concentration(s) is/are the result of interferon receptor agonist dosage amounts, delivery rates and dosage schedules specified by the set of instructions without user input.

This example of the invention also includes embodiments where the initial and/or sustained serum concentrations(s) of the interferon receptor agonist is/are selected by the user. The apparatus can be designed to allow the user to select a serum concentration parameter from a fixed set of values specified by the set of instructions. Alternatively, the system can permit the user to select any serum concentration within a range of values specified by the set of instructions. In these embodiments, the set of instructions can be designed to calculate and cause the device to utilize appropriate interferon receptor agonist dosage amounts, delivery rates and dosage schedules for the implementation of the user-specified serum concentration(s), the particular delivery device, the selected route of administration, and the course of treatment to be employed.

This example of the invention further includes embodiments where the duration(s) of the initial dosage phase and/or sustained dosage interval(s) or the duration of the interferon receptor agonist therapy is/are dictated by the set of instructions without user input.

This example of the invention also includes embodiments where the user is allowed to set the apparatus for the treatment of a patient with particular characteristics, e.g., the genotype of the HCV infection of the patient, the initial viral load of the patient, the antiviral treatment history of the patient, and the like, and the set of instructions adopts a particular duration or durations for the initial dosage phase and/or sustained dosage interval(s) or a duration for the interferon receptor agonist therapy based on the pattern of patient characteristics indicated in the user's setting.

This example of the invention additionally includes embodiments where the user is allowed to select the duration(s) of the initial dosage phase and/or sustained dosage interval(s) or any duration of the interferon receptor agonist therapy from a fixed set of values specified by the set of instructions.

This example of the invention also encompasses embodiments where the user is allowed to select any duration for the initial dosage phase and/or sustained dosage interval(s) or any duration of the interferon receptor agonist therapy within a fixed range or ranges specified by the set of instructions.

In another aspect, the invention provides an apparatus for the controlled delivery of an interferon receptor agonist to a patient having an HCV infection, where (i) the apparatus includes a device for delivery of the interferon receptor agonist to the patient by a selected route of administration, (ii) the apparatus includes a control unit that is operated by a series of commands, (iii) the series of commands contains a set of instructions that causes the device to administer a therapeutically effective amount of the interferon receptor agonist to the patient via the selected route of administration in an initial dosage phase followed by a sustained dosage phase consisting of at least one sustained dosage interval, where the initial dosage phase extends for a period of time of about 12 hours to about 48 hours and an initial pre-selected amount of the interferon receptor agonist is administered to the individual during the initial dosage phase, where during the first sustained dosage interval a first sustained pre-selected amount of the interferon receptor agonist is administered to the individual each day in a substantially continuous or continuous manner, where the first sustained pre-selected amount of the interferon receptor agonist is at least about 80% and up to about 200% of the initial pre-selected amount of the interferon receptor agonist, and during any following sustained dosage interval a following sustained pre-selected amount of the interferon receptor agonist is administered to the individual each day in a substantially continuous or continuous manner, where the following sustained pre-selected amount of the interferon receptor agonist is at least about 20% of the first sustained pre-selected amount of the interferon receptor agonist and at least about 50% and up to about 200% of the sustained pre-selected amount of the interferon receptor agonist in the preceding sustained dosage interval, where each sustained dosage interval extends for a period of time of at least about 5 days, and where the duration of the interferon receptor agonist therapy extends for a period of time of at least about 6 weeks, or at least about 12 weeks, or at least about 24 weeks, or at least about 48 weeks, or at least about 60 weeks, and (iv) when the apparatus is in place and operational on the patient, the control unit executes the set of instructions in the series of commands.

This example of the invention includes embodiments where any pre-selected amount of interferon receptor agonist is specified by the set of instructions without user input. In other embodiments, the apparatus can be designed to allow the user to select any pre-selected amount of the interferon receptor agonist from a fixed set of values specified by the set of instructions. Alternatively, the apparatus can permit the user to select any pre-selected amount of interferon receptor agonist within a range of values specified by the set of instructions.

This example of the invention further includes embodiments where the duration(s) of the initial dosage phase and/or sustained dosage interval(s) or duration of the interferon receptor agonist therapy is/are dictated by the set of instructions without user input.

This example of the invention also includes embodiments where the user is allowed to set the apparatus for the treatment of a patient with particular characteristics, e.g., the genotype of the HCV infection of the patient, the initial viral load of the patient, the antiviral treatment history of the patient, and the like, and the set of instructions adopts a particular duration or durations of treatment for the initial dosage phase and/or sustained dosage interval(s) or duration of the interferon receptor agonist therapy based on the pattern of patient characteristics indicated in the user's setting.

This example of the invention additionally includes embodiments where the user is allowed to select the duration(s) of the initial dosage phase and/or sustained dosage interval(s) or duration of the interferon receptor agonist therapy from a fixed set of values specified by the set of instructions.

This example of the invention also encompasses embodiments where the user is allowed to select any duration for the initial dosage phase and/or sustained dosage interval(s) or duration of the interferon receptor agonist therapy within a fixed range or ranges specified by the set of instructions.

It will be understood that in the embodiments of the invention that allow user input, the invention contemplates the use of any interface for user input that permits the user to set the apparatus as desired. For example, the apparatus of the invention can employ an interactive, computer-controlled interface that prompts the user for input. Alternatively, the apparatus of the invention can employ a manual, switch-operated interface that requires the user to (1) match a particular pattern of patient characteristics and/or treatment parameters with a particular switch setting for the apparatus and (2) manually deploy the particular switch setting.

It will be understood that the apparatus of the invention can be made or practiced with (1) any device for the controlled delivery of an interferon receptor agonist (e.g., any of the devices described above) (2) any route of administration suitable for delivery of the interferon receptor agonist to the patient by the delivery device and (3) any set of instructions that causes the device to administer the interferon receptor agonist to the patient by the route of administration for the treatment of HCV infection in the patient according to any method described herein.

As described in more detail below, in any of the above-described apparatus, the interferon receptor agonist is a Type I interferon receptor agonist, a Type II interferon receptor agonist, or a Type III interferon receptor agonist. In particular embodiments, in any of the above-described apparatus, the interferon receptor agonist is an IFN-α. In particular embodiments, in any of the above-described apparatus, the interferon receptor agonist is a CIFN. In particular embodiments, in any of the above-described apparatus, the interferon receptor agonist is INFERGEN® interferon alfacon-1. In other particular embodiments, in any of the above-described apparatus, the interferon receptor agonist is IFN-α 2a or IFN-α 2b. In preferred embodiments, where the interferon receptor agonist is an IFN-α, the IFN-α administered to the patient according to the methods of the invention is an unPEGylated IFN-α.

### Agonists of Type I Interferon Receptors

In any of the above-described methods or apparatus, the interferon receptor agonist is in some embodiments an agonist of a Type I interferon receptor (e.g., “a Type I interferon receptor agonist”). As used herein, a Type I interferon receptor agonist is any naturally occurring or non-naturally occurring ligand of the human Type I interferon receptor that binds to and causes signal transduction via the receptor. Type I interferon receptor agonists include an IFN-α; an IFN-β; an IFN-tau; an IFN-ω; antibody agonists specific for a Type I interferon receptor; and any other agonist of Type I interferon receptor, including non-polypeptide agonists.

### IFN-α

The term “interferon-alpha” (IFN-α) as used herein refers to a family of related polypeptides that inhibit viral replication and cellular proliferation and modulate immune response. The term “IFN-α” includes IFN-α polypeptides that are naturally occurring; non-naturally-occurring IFN-α polypeptides; and analogs of naturally occurring or non-naturally occurring IFN-α that retain antiviral activity of a parent naturally-occurring or non-naturally occurring IFN-α.

Any of a variety of alpha interferons can be delivered by the continuous delivery method of the present invention. Suitable alpha interferons include, but are not limited to, naturally-occurring IFN-α (including, but not limited to, naturally occurring IFN-α2a, IFN-α2b); recombinant interferon alpha-2b such as Intron® A interferon available from Schering Corporation, Kenilworth, N.J.; recombinant interferon alpha-2a such as Roferon® interferon available from Hoffmann-La Roche, Nutley, N.J.; recombinant interferon alpha-2C such as Berofor® alpha 2 interferon available from Boehringer Ingelheim Pharmaceutical, Inc., Ridgefield, Conn.; interferon alpha-n1, a purified blend of natural alpha interferons such as Sumiferon available from Sumitomo, Japan or as Wellferon® interferon alpha-n1 (INS) available from the Glaxo-Wellcome Ltd., London, Great Britain; and interferon alpha-n3 a mixture of natural alpha interferons made by Interferon Sciences and available from the Purdue Frederick Co., Norwalk, Conn., under the Alferon® trademark.

The IFN-α formulation may comprise an N-blocked species, wherein the N-terminal amino acid is acylated with an acyl group, such as a formyl group, an acetyl group, a malonyl group, and the like.

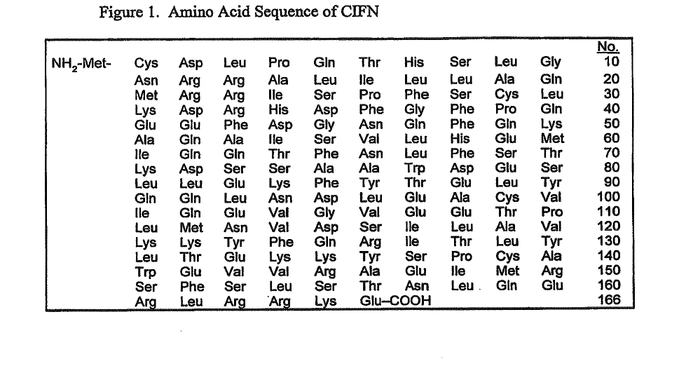

The term “IFN-α,” as used herein, also encompasses consensus IFN-α. As used herein, the term “consensus IFN-α” refers to a non-naturally-occurring polypeptide, which includes those amino acid residues that are common to all naturally-occurring human leukocyte IFN-α subtype sequences and which includes, at one or more of those positions where there is no amino acid common to all subtypes, an amino acid which predominantly occurs at that position, provided that at any such position where there is no amino acid common to all subtypes, the polypeptide excludes any amino acid residue which is not present in at least one naturally-occurring subtype. Amino acid residues that are common to all naturally-occurring human leukocyte IFN-α subtype sequences (“common amino acid residues”), and amino acid residues that occur predominantly at non-common residues (“consensus amino acid residues”) are known in the art. See FIG. 1 for the amino acid sequence of IFN-alpha con1.

Consensus IFN-α (also referred to as “CIFN” and “IFN-con” and “IFN-alpha con”) encompasses but is not limited to the amino acid sequences designated IFN-con₁ (sometimes referred to as “CIFN-alpha con1,” “IFN-alpha con1,” or “IFN-con1,” or “alphacon-1”), IFN-con₂ and IFN-con₃, which are disclosed in U.S. Pat. Nos. 4,695,623 and 4,897,471; and Infergen® (InterMune, Inc., Brisbane, Calif.). Consensus interferons are generally defined by determination of a consensus sequence of naturally occurring interferon alphas. PEG-modified CIFN, especially Infergen®, is of particular interest in some embodiments.

Also suitable for use in the present invention are fusion polypeptides comprising an IFN-α and a heterologous polypeptide. Suitable IFN-α fusion polypeptides include, but are not limited to, Albuferon-alpha™ (a fusion product of human albumin and IFN-α; Human Genome Sciences; see, e.g., Osborn et al. (2002) J. Pharmacol. Exp. Therap. 303:540-548). Also suitable for use in the present invention are gene-shuffled forms of IFN-α. See, e.g., Masci et al. (2003) Curr. Oncol. Rep. 5:108-113.

IFN-α polypeptides can be produced by any known method. DNA sequences encoding IFN-con may be synthesized as described in the above-mentioned patents or other standard methods. In many embodiments, IFN-α polypeptides are the products of expression of manufactured DNA sequences transformed or transfected into bacterial hosts, e.g., E. coli, or in eukaryotic host cells (e.g., yeast; mammalian cells, such as CHO cells; and the like). In these embodiments, the IFN-α is “recombinant IFN-α.” Where the host cell is a bacterial host cell, the IFN-α is modified to comprise an N-terminal methionine. IFN-α produced in E. coli is generally purified by procedures known to those skilled in the art and generally described in Klein et al. ((1988) J. Chromatog. 454:205-215) for IFN-con₁.

Bacterially produced IFN-α may comprise a mixture of isoforms with respect to the N-terminal amino acid residue. For example, purified IFN-con may comprise a mixture of isoforms with respect to the N-terminal methionine status. For example, in some embodiments, an IFN-con comprises a mixture of N-terminal methionyl IFN-con, des-methionyl IFN-con with an unblocked N-terminus, and des-methionyl IFN-con with a blocked N-terminus. As one non-limiting example, purified IFN-con₁ comprises a mixture of methionyl IFN-con₁ des-methionyl IFN-con₁ and des-methionyl IFN-con₁ with a blocked N-terminus. Klein et al. ((1990) Arch Biochemistry & Biophys. 276:531-537). Alternatively, IFN-con may comprise a specific, isolated isoform. Isoforms of IFN-con are separated from each other by techniques such as isoelectric focusing which are known to those skilled in the art.

It is to be understood that IFN-α as described herein may comprise one or more modified amino acid residues, e.g., glycosylations, chemical modifications, and the like.

### IFN-β

The term interferon-beta (“IFN-β”) includes IFN-β polypeptides that are naturally occurring; non-naturally-occurring IFN-β polypeptides; and analogs of naturally occurring or non-naturally occurring IFN-β that retain antiviral activity of a parent naturally-occurring or non-naturally occurring IFN-β.

Any of a variety of beta interferons can be delivered by the continuous delivery method of the present invention. Suitable beta interferons include, but are not limited to, naturally-occurring IFN-β; IFN-β1a, e.g., Avonex® (Biogen, Inc.), and Rebif® (Serono, SA); IFN-β1b (Betaseron®; Berlex); and the like.

The IFN-β formulation may comprise an N-blocked species, wherein the N-terminal amino acid is acylated with an acyl group, such as a formyl group, an acetyl group, a malonyl group, and the like. Also suitable for use is a consensus IFN-β.

IFN-β polypeptides can be produced by any known method. DNA sequences encoding IFN-β may be synthesized using standard methods. In many embodiments, IFN-β polypeptides are the products of expression of manufactured DNA sequences transformed or transfected into bacterial hosts, e.g., E. coli, or in eukaryotic host cells (e.g., yeast; mammalian cells, such as CHO cells; and the like). In these embodiments, the IFN-β is “recombinant IFN-α.”Where the host cell is a bacterial host cell, the IFN-β is modified to comprise an N-terminal methionine.

It is to be understood that IFN-β as described herein may comprise one or more modified amino acid residues, e.g., glycosylations, chemical modifications, and the like.

### IFN-tau

The term interferon-tau includes IFN-tau polypeptides that are naturally occurring; non-naturally-occurring IFN-tau polypeptides; and analogs of naturally occurring or non-naturally occurring IFN-tau that retain antiviral activity of a parent naturally-occurring or non-naturally occurring IFN-tau.

Suitable tau interferons include, but are not limited to, naturally-occurring IFN-tau; Tauferon® (Pepgen Corp.); and the like.

The IFN-tau formulation may comprise an N-blocked species, wherein the N-terminal amino acid is acylated with an acyl group, such as a formyl group, an acetyl group, a malonyl group, and the like. Also suitable for use is a consensus IFN-tau.

IFN-tau polypeptides can be produced by any known method. DNA sequences encoding IFN-tau may be synthesized using standard methods. In many embodiments, IFN-tau polypeptides are the products of expression of manufactured DNA sequences transformed or transfected into bacterial hosts, e.g., E. coli or in eukaryotic host cells (e.g., yeast; mammalian cells, such as CHO cells; and the like). In these embodiments, the IFN-tau is “recombinant IFN-α.” Where the host cell is a bacterial host cell, the IFN-tau is modified to comprise an N-terminal methionine.

It is to be understood that IFN-tau as described herein may comprise one or more modified amino acid residues, e.g., glycosylations, chemical modifications, and the like.

### IFN-ω

The term interferon-omega (“IFN-ω”) includes IFN-ω polypeptides that are naturally occurring; non-naturally-occurring IFN-ω polypeptides; and analogs of naturally occurring or non-naturally occurring IFN-ω that retain antiviral activity of a parent naturally-occurring or non-naturally occurring IFN-ω.

Any known omega interferon can be delivered by the continuous delivery method of the present invention. Suitable IFN-ω) include, but are not limited to, naturally-occurring IFN-ω; recombinant IFN-ω, e.g., Biomed 510 (BioMedicines); and the like.

IFN-ω may comprise an amino acid sequence as set forth in GenBank Accession No. NP_{—}002168; or AAA70091. The sequence of any known IFN-ω polypeptide may be altered in various ways known in the art to generate targeted changes in sequence. A variant polypeptide will usually be substantially similar to the sequences provided herein, i.e. will differ by at least one amino acid, and may differ by at least two but not more than about ten amino acids. The sequence changes may be substitutions, insertions or deletions. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation; and the like. Also included are modifications of glycosylation, e.g. those made by modifying the glycosylation patterns of a polypeptide during its synthesis and processing or in further processing steps; e.g. by exposing the polypeptide to enzymes that affect glycosylation, such as mammalian glycosylating or deglycosylating enzymes. Also embraced are sequences that have phosphorylated amino acid residues, e.g. phosphotyrosine, phosphoserine, or phosphothreonine.

The IFN-ω formulation may comprise an N-blocked species, wherein the N-terminal amino acid is acylated with an acyl group, such as a formyl group, an acetyl group, a malonyl group, and the like. Also suitable for use is a consensus IFN-ω).

IFN-ω polypeptides can be produced by any known method. DNA sequences encoding IFN-ω may be synthesized using standard methods. In many embodiments, IFN-ω polypeptides are the products of expression of manufactured DNA sequences transformed or transfected into bacterial hosts, e.g., E. coli, or in eukaryotic host cells (e.g., yeast; mammalian cells, such as CHO cells; and the like). In these embodiments, the IFN-ω is “recombinant IFN-ω.” Where the host cell is a bacterial host cell, the IFN-ω is modified to comprise an N-terminal methionine.

It is to be understood that IFN-ω as described herein may comprise one or more modified amino acid residues, e.g., glycosylations, chemical modifications, and the like.

### Agonists of Type II Interferon Receptors

In any of the above-described methods or apparatus, the interferon receptor agonist is in some embodiments an agonist of a Type II interferon receptor (e.g., “a Type II interferon agonist”). As used herein, a Type II interferon receptor agonist is any naturally occurring or non-naturally occurring ligand of the human Type II interferon receptor that binds to and causes signal transduction via the receptor. Type I interferon receptor agonists include an IFN-γ; antibody agonists specific for Type II interferon receptor; and any other agonist of Type II interferon receptor, including non-polypeptide agonists.

### IFN-γ

The nucleic acid sequences encoding IFN-γ polypeptides may be accessed from public databases, e.g. Genbank, journal publications, etc. While various mammalian IFN-γ polypeptides are of interest, for the treatment of human disease, generally the human protein will be used. Human IFN-γ coding sequence may be found in Genbank, accession numbers X13274; V00543; and NM_{—}000619. The corresponding genomic sequence may be found in Genbank, accession numbers J00219; M37265; and V00536. See, for example. Gray et al. (1982) Nature 295:501 (Genbank X13274); and Rinderknecht et al. (1984) J.B.C. 259:6790.

IFN-γ1b (Actimmune®); human interferon) is a single-chain polypeptide of 140 amino acids. It is made recombinantly in E. coli and is unglycosylated. Rinderknecht et al. (1984) J. Biol. Chem. 259:6790-6797.

The IFN-γ to be used in the compositions of the present invention may be any of natural IFN-γs, recombinant IFN-γs and the derivatives thereof so far as they have a IFN-γ activity, particularly human IFN-γ activity. Human IFN-γ exhibits the antiviral and anti-proliferative properties characteristic of the interferons, as well as a number of other immunomodulatory activities, as is known in the art. Although IFN-γ is based on the sequences as provided above, the production of the protein and proteolytic processing can result in processing variants thereof. The unprocessed sequence provided by Gray et al., supra. consists of 166 amino acids (aa). Although the recombinant IFN-γ produced in E. coli was originally believed to be 146 amino acids, (commencing at amino acid 20) it was subsequently found that native human IFN-γ is cleaved after residue 23, to produce a 143 aa protein, or 144 aa if the terminal methionine is present, as required for expression in bacteria. During purification, the mature protein can additionally be cleaved at the C terminus after reside 162 (referring to the Gray et al. sequence), resulting in a protein of 139 amino acids, or 140 amino acids if the initial methionine is present, e.g. if required for bacterial expression. The N-terminal methionine is an artifact encoded by the mRNA translational “start” signal AUG which, in the particular case of E. coli expression is not processed away. In other microbial systems or eukaryotic expression systems, methionine may be removed.

For use in the subject methods, any of the native IFN-γ peptides, modifications and variants thereof, or a combination of one or more peptides may be used. IFN-γ peptides of interest include fragments, and can be variously truncated at the carboxy terminal end relative to the full sequence. Such fragments continue to exhibit the characteristic properties of human gamma interferon, so long as amino acids 24 to about 149 (numbering from the residues of the unprocessed polypeptide) are present. Extraneous sequences can be substituted for the amino acid sequence following amino acid 155 without loss of activity. See, for example, U.S. Pat. No. 5,690,925, herein incorporated by reference. Native IFN-γ moieties include molecules variously extending from amino acid residues 24-150; 24-151, 24-152; 24-153, 24-155; and 24-157. Any of these variants, and other variants known in the art and having IFN-γ activity, may be used in the present methods.

The sequence of the IFN-γ polypeptide may be altered in various ways known in the art to generate targeted changes in sequence. A variant polypeptide will usually be substantially similar to the sequences provided herein, i.e. will differ by at least one amino acid, and may differ by at least two but not more than about ten amino acids. The sequence changes may be substitutions, insertions or deletions. Scanning mutations that systematically introduce alanine, or other residues, may be used to determine key amino acids. Specific amino acid substitutions of interest include conservative and non-conservative changes. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation; and the like. Also included are modifications of glycosylation, e.g. those made by modifying the glycosylation patterns of a polypeptide during its synthesis and processing or in further processing steps; e.g. by exposing the polypeptide to enzymes that affect glycosylation, such as mammalian glycosylating or deglycosylating enzymes. Also embraced are sequences that have phosphorylated amino acid residues, e.g. phosphotyrosine, phosphoserine, or phosphothreonine.

Included in the subject invention are polypeptides that have been modified using ordinary chemical techniques so as to improve their resistance to proteolytic degradation, to optimize solubility properties, or to render them more suitable as a therapeutic agent. For examples, the backbone of the peptide may be cyclized to enhance stability (see Friedler et al. (2000) J. Biol. Chem. 275:23783-23789). Analogs may be used that include residues other than naturally occurring L-amino acids, e.g. D-amino acids or non-naturally occurring synthetic amino acids. The protein may be pegylated to enhance stability.

The polypeptides may be prepared by in vitro synthesis, using conventional methods as known in the art, by recombinant methods, or may be isolated from cells induced or naturally, producing the protein. The particular sequence and the manner of preparation will be determined by convenience, economics, purity required, and the like. If desired, various groups may be introduced into the polypeptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

### Agonists of Type III Interferon Receptors

In any of the above-described methods or apparatus, the interferon receptor agonist is in some embodiments an agonist of a Type III interferon receptor (e.g., “a Type III interferon receptor agonist”). As used herein, a Type III interferon receptor agonist is defined as any ligand of the human IL-28 receptor α (“IL-28R”; the amino acid sequence of which was reported by Sheppard, et al., Nat. Immunol., 4: 63-68 (2003)) that binds to and causes signal transduction via the receptor. Type III interferon agonists include an IL-28b polypeptide; and IL-28a polypeptide; and IL-29 polypeptide; antibody specific for a Type III interferon receptor; and any other agonist of Type III interferon receptor, including non-polypeptide agonists.

IL-28A, IL-28B, and IL-29 (referred to herein collectively as “Type III interferons” or “Type III IFNs”) are described in Sheppard et al. (2003) Nature 4:63-68. Each polypeptide binds a heterodimeric receptor consisting of IL-10 receptor P chain and an IL-28 receptor α. Sheppard et al. (2003), supra. The amino acid sequences of IL-28A, IL-28B, and IL-29 are found under GenBank Accession Nos. NP_{—}742150, NP_{—}742151, and NP_{—}742152, respectively.

The amino acid sequence of a Type III IFN polypeptide may be altered in various ways known in the art to generate targeted changes in sequence. A variant polypeptide will usually be substantially similar to the sequences provided herein, i.e. will differ by at least one amino acid, and may differ by at least two but not more than about ten amino acids. The sequence changes may be substitutions, insertions or deletions. Scanning mutations that systematically introduce alanine, or other residues, may be used to determine key amino acids. Specific amino acid substitutions of interest include conservative and non-conservative changes. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation; and the like. Also included are modifications of glycosylation, e.g. those made by modifying the glycosylation patterns of a polypeptide during its synthesis and processing or in further processing steps; e.g. by exposing the polypeptide to enzymes that affect glycosylation, such as mammalian glycosylating or deglycosylating enzymes. Also embraced are sequences that have phosphorylated amino acid residues, e.g. phosphotyrosine, phosphoserine, or phosphothreonine.

Included in the subject invention are polypeptides that have been modified using ordinary chemical techniques so as to improve their resistance to proteolytic degradation, to optimize solubility properties, or to render them more suitable as a therapeutic agent. For examples, the backbone of the peptide may be cyclized to enhance stability (see Friedler et al. (2000) J. Biol. Chem. 275:23783-23789). Analogs may be used that include residues other than naturally occurring L-amino acids, e.g. D-amino acids or non-naturally occurring synthetic amino acids. The protein may be pegylated to enhance stability. The polypeptides may be fused to albumin.

The polypeptides may be prepared by in vitro synthesis, using conventional methods as known in the art, by recombinant methods, or may be isolated from cells induced or naturally producing the protein. The particular sequence and the manner of preparation will be determined by convenience, economics, purity required, and the like. If desired, various groups may be introduced into the polypeptide during synthesis or during expression, which allow for lining to other molecules or to a surface. Thus cysteines can be used to make thioethers, histidines for linking to a metal ion complex, carboxyl groups for forming amides or esters, amino groups for forming amides, and the like.

### Formulations

The above-discussed compositions can be formulated using well-known reagents and methods. Compositions are provided in formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, an alpha-interferon is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

### Dosages

Appropriate dosages of an interferon receptor agonist are readily determined by those skilled in the art.

Effective dosages of an IFN-α can range from 0.5 μg to about 30 μg, e.g., exemplary effective dosages of an IFN-α are at least about 0.5 μg, or at least about 1.0 μg, or at least about 1.5 μg, or at least about 2.0 μg, or at least about 2.5 μg, or at least about 3 μg, or at least about 6 μg, or at least about 9 μg, or at least about 12 μg, or at least about 15 μg, or at least about 18 μg, or at least about 21 μg, or at least about 24 μg, or at least about 27 μg, or at least about 30 μg.

Effective dosages of an IFN-β can range from 3 μg to about 50 μg. Exemplary effective dosages of an IFN-β are 30 μg, and 44 μg.

Effective dosages of IFN-γ can range from about 25 μg/dose to about 300 μg/dose.

### Combination Therapies

In some aspects, the invention features methods for combination therapy comprising administering an interferon receptor agonist and an additional therapeutic agent such as ribavirin and/or pirfenidone or pirfenidone analog. For example, the invention provides any of the above-described methods in which the interferon receptor agonist is a Type I interferon receptor agonist and the subject method comprises co-administration of an effective amount of pirfenidone or pirfenidone analog for the duration of the Type I interferon receptor agonist therapy in the subject method.

In another example, the invention provides any of the above-described methods in which the interferon receptor agonist is a Type I interferon receptor agonist and the subject method comprises co-administration of pirfenidone or pirfenidone analog for the duration of the Type I interferon receptor agonist therapy in an amount that is synergistically effective with the Type I interferon receptor agonist therapy in the subject method.

In another example, the invention provides any of the above-described methods in which the interferon receptor agonist is a Type II interferon receptor agonist and the subject method comprises co-administration of an effective amount of pirfenidone or pirfenidone analog for the duration of the Type II interferon receptor agonist therapy in the subject method.

In another example, the invention provides any of the above-described methods in which the interferon receptor agonist is a Type II interferon receptor agonist and the subject method comprises co-administration of pirfenidone or pirfenidone analog for the duration of the Type II interferon receptor agonist therapy in an amount that is synergistically effective with the Type II interferon receptor agonist therapy in the subject method.

In another example, the invention provides any of the above-described methods in which the interferon receptor agonist is a Type III interferon receptor agonist and the subject method comprises co-administration of an effective amount of pirfenidone or pirfenidone analog for the duration of the Type III interferon receptor agonist therapy in the subject method.

In another example, the invention provides any of the above-described methods in which the interferon receptor agonist is a Type III interferon receptor agonist and the subject method comprises co-administration of pirfenidone or pirfenidone analog for the duration of the Type III interferon receptor agonist therapy in an amount that is synergistically effective with the Type III interferon receptor agonist therapy in the subject method.

In another example, the invention provides any of the above-described methods in which the interferon receptor agonist is an IFN-α and the subject method comprises co-administration of an effective amount of pirfenidone or pirfenidone analog for the duration of the IFN-α therapy in the subject method.

In another example, the invention provides any of the above-described methods in which the interferon receptor agonist is an IFN-α and the subject method comprises co-administration of pirfenidone or pirfenidone analog for the duration of the IFN-α therapy in an amount that is synergistically effective with the IFN-M therapy in the subject method.

In one embodiment, the IFN-α is a consensus interferon (CIFN) and the subject method co-administers to the patient a synergistically effective amount of about 0.5 μg to about 30 μg of CIFN per day and about 50 mg to about 5,000 mg pirfenidone or specific pirfenidone analog orally per day.

In another embodiment, the IFN-α is a consensus interferon (CIFN) and the subject method co-administers to the patient a synergistically effective amount of about 1 μg to about 10 μg of CIFN per day and about 100 mg to about 1,000 mg pirfenidone or specific pirfenidone analog orally per day.

In another embodiment, the IFN-α is consensus interferon (CIFN) and the subject method co-administers to the patient a synergistically effective amount of about 9 μg of CIFN per day and about 500 mg pirfenidone or specific pirfenidone analog orally per day.

In another aspect, the invention provides any of the above-described methods for co-administration of an interferon receptor agonist and pirfenidone or pirfenidone analog in which the subject method further comprises co-administration of an effective amount of ribavirin. For example, the methods of the invention encompass co-administering to the patient 800 mg to about 1200 mg ribavirin orally per day. In another example the methods of the invention encompass co-administering to the patient (a) 1000 mg ribavirin orally per day if the patient has a body weight less than 75 kg or (b) 1200 mg ribavirin orally per day if the patient has a body weight of greater than or equal to 75 kg.

In other aspects, the invention features methods for combination therapy comprising administering a Type I interferon receptor agonist and a Type II interferon receptor agonist. For example, the invention provides any of the above-described methods in which the interferon receptor agonist is an IFN-α and the subject method comprises co-administration of an effective amount of IFN-γ for the duration of the IFN-α therapy in the subject method.

In another example, the invention provides any of the above-described methods in which the interferon receptor agonist is an IFN-α and the subject method comprises co-administration of IFN-γ for the duration of the IFN-α therapy in the subject method in an amount that is synergistically effective with the IFN-α therapy provided in the subject method.

In one embodiment, the IFN-α is consensus interferon (CIFN) and the subject method co-administers to the patient a synergistically effective amount of about 0.5 μg to about 30 μg of CIFN per day and about 5 μg to about 300 μg of IFN-γ per day.

In another embodiment, the IFN-α is consensus interferon (CIFN) and the subject method co-administers to the patient a synergistically effective amount of about 1 μg of CIFN per day and about 10 μg to about 50 μg of IFN-γ per day.

In another embodiment, the IFN-γ is consensus interferon (CIFN) and the subject method co-administers to the patient a synergistically effective amount of about 9 μg of CIFN per day and about 90 μg to about 100 μg of IFN-γ per day.

In another embodiment, the IFN-α is consensus interferon (CIFN) and the subject method co-administers to the patient a synergistically effective amount of about 30 μg of CIFN per day and about 200 μg to about 300 μg of IFN-γ per day.

In another example, the IFN-α is IFN-α 2a or 2b or 2c and the subject method co-administers to the patient a synergistically effective amount of about 0.5 million units (MU) to about 20 MU of IFN-α 2a or 2b or 2c per day and about 15 μg to about 600 μg of IFN-γ per day.

In another example, the IFN-A is IFN-α 2a or 2b or 2c and the subject method co-administers to the patient a synergistically effective amount of about 1 million units (MU) to about 20 MU of IFN-α 2a or 2b or 2c per day and about 30 μg to about 600 μg of IFN-γ per day.

In another example, the IFN-α is IFN-α 2a or 2b or 2c and the subject method co-administers to the patient a synergistically effective amount of about 3 million units (MU) of IFN-α 2a or 2b or 2c per day and about 100 μg of IFN-γ per day.

In another example, the IFN-α is IFN-α 2a or 2b or 2c and the subject method co-administers to the patient a synergistically effective amount of about 10 million units (MU) of IFN-α 2a or 2b or 2c per day and about 300 μg of IFN-γ per day.

In some embodiments, the methods provide for combination therapy comprising administering a Type I interferon receptor agonist and a Type III interferon receptor agonist. In some embodiments, the methods provide for combination therapy comprising administering a Type II interferon receptor agonist and a Type III interferon receptor agonist.

In another aspect, the invention provides any of the above-described methods for co-administration of two or more different interferon receptor agonists in which the subject method further comprises co-administration of an effective amount of pirfenidone or pirfenidone analog for the duration of the interferon receptor agonist combination therapy. For example, the invention provides any of the above-described methods comprising co-administration of IFN-α and IFN-γ and further comprising co-administration of an effective amount of pirfenidone or a pirfenidone analog for the duration of the IFN-α and IFN-γ combination therapy in the subject method.

In another example, the invention provides any of the above-described methods in which the subject method comprises co-administration of two or more interferon receptor agonists and further comprises co-administration of pirfenidone or pirfenidone analog for the duration of the interferon receptor agonist combination therapy in an amount that is synergistically effective with the interferon receptor agonist combination therapy in the subject method.

In another example, the invention provides any of the above-described methods comprising co-administration of IFN-α and IFN-γ and further comprising co-administration of pirfenidone or a pirfenidone analog for the duration of the IFN-α and IFN-γ combination therapy in an amount that is synergistically effective with the IFN-α and IFN-γ combination therapy in the subject method. In some embodiments, the subject method provides for co-administering to the patient about 50 mg to about 5,000 mg pirfenidone or specific pirfenidone analog orally per day. In other embodiments, the subject method provides for co-administering to the patient about 100 mg to about 1,000 mg pirfenidone or specific pirfenidone analog orally per day. In further embodiments, the subject method provides for co-administering to the patient about 500 mg pirfenidone or specific pirfenidone analog orally per day.

In another example, the invention provides any of the above-described methods comprising co-administration of IFN-t and IFN-γ and further comprising co-administration of pirfenidone or a pirfenidone analog for the duration of the IFN-α and IFN-γ combination therapy in an amount that reduces side effects induced by the IFN-α and IFN-γ combination therapy in the subject method. In some embodiments, the subject method provides for co-administering to the patient about 1,000 mg to about 10,000 mg pirfenidone or specific pirfenidone analog orally per day. In other embodiments, the subject method provides for co-administering to the patient about 1,000 mg to about 3,000 mg pirfenidone or specific pirfenidone analog orally per day. In further embodiments, the subject method provides for co-administering to the patient about 1,000 mg to about 2,000 mg pirfenidone or specific pirfenidone analog orally per day.

In another aspect, the invention provides any of the above-described methods for co-administration of two or more different interferon receptor agonists and pirfenidone in which the subject method further comprises co-administration of an effective amount of ribavirin for the duration of the interferon receptor agonist combination therapy. For example, the invention provides any of the above-described methods comprising co-administration of IFN-α and IFN-γ and pirfenidone or pirfenidone analog and further comprising co-administration of an effective amount of ribavirin for the duration of the IFN-α and IFN-γ combination therapy in the subject method. In some embodiments, the subject method provides for co-administering to the patient 800 mg to about 1200 mg ribavirin orally per day. In other embodiments, the subject method provides for co-administering to the patient (a) 1000 mg ribavirin orally per day if the patient has a body weight less than 75 kg or (b) 1200 mg ribavirin orally per day if the patient has a body weight of greater than or equal to 75 kg.

### Combination Therapy: Interferon Receptor Agonist and an Additional Therapeutic Agent

In some embodiments, the additional therapeutic agent(s) is administered during the entire course of interferon receptor agonist treatment, and the beginning and end of the treatment periods coincide. In other embodiments, the additional therapeutic agent(s) is administered for a period of time that is overlapping with that of the interferon receptor agonist treatment, e.g., treatment with the additional therapeutic agent(s) begins before the interferon receptor agonist treatment begins and ends before the interferon receptor agonist treatment ends; treatment with the additional therapeutic agent(s) begins after the interferon receptor agonist treatment begins and ends after the interferon receptor agonist treatment ends; treatment with the additional therapeutic agent(s) begins after the interferon receptor agonist treatment begins and ends before the interferon receptor agonist treatment ends; or treatment with the additional therapeutic agent(s) begins before the interferon receptor agonist treatment begins and ends after the interferon receptor agonist treatment ends.

An interferon receptor agonist can be administered together with (i.e., simultaneously in separate formulations; simultaneously in the same formulation; administered in separate formulations and within about 48 hours, within about 36 hours, within about 24 hours, within about 16 hours, within about 12 hours, within about 8 hours, within about 4 hours, within about 2 hours, within about 1 hour, within about 30 minutes, or within about 15 minutes or less) one or more additional therapeutic agents.

### Ribavirin and Other Antiviral Agents

Ribavirin, 1-β-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. The invention also contemplates use of derivatives of ribavirin (see, e.g., U.S. Pat. No. 6,277,830). Ribavirin is administered in dosages of about 400, about 800, or about 1200 mg per day.

In one embodiment, the invention provides any of the above-described methods modified to include co-administering to the patient a therapeutically effective amount of ribavirin for the duration of the desired course of interferon receptor agonist treatment.

In another embodiment, the invention provides any of the above-described methods modified to include co-administering to the patient about 800 mg to about 1200 mg ribavirin orally per day for the duration of the desired course of interferon receptor agonist treatment.

In another embodiment, the invention provides any of the above-described methods modified to include co-administering to the patient (a) 1000 mg ribavirin orally per day if the patient has a body weight less than 75 kg or (b) 1200 mg ribavirin orally per day if the patient has a body weight greater than or equal to 75 kg, where the daily dosage of ribavirin is optionally divided into to 2 doses for the duration of the desired course of interferon receptor agonist treatment.

Other antiviral agents can be delivered in the treatment methods of the invention. For example, compounds that inhibit inosine monophosphate dehydrogenase (IMPDH) may have the potential to exert direct anti viral activity, and such compounds can be administered in combination with an interferon receptor agonist composition, as described herein. Drugs that are effective inhibitors of hepatitis C NS3 protease may be administered in combination with an interferon receptor agonist a composition, as described herein. Hepatitis C NS3 protease inhibitors inhibit viral replication. Other agents such as inhibitors of HCV NS3 helicase are also attractive drugs for combinational therapy, and are contemplated for use in combination therapies described herein. Ribozymes such as Heptazyme™ and phosphorothioate oligonucleotides which are complementary to HCV protein sequences and which inhibit the expression of viral core proteins are also suitable for use in combination therapies described herein.

### Pirfenidone and Analogs Thereof

Pirfenidone (5-methyl-1-phenyl-2-(1H)pyridone) and specific pirfenidone analogs are disclosed for the treatment of fibrotic conditions. A “fibrotic condition” is one that is amenable to treatment by administration of a compound having anti-fibrotic activity.

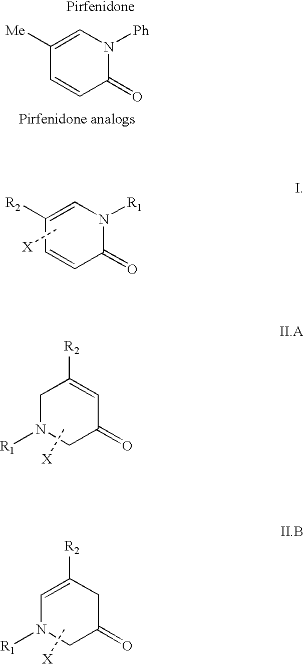

### Descriptions for Substituents R₁, R₂, X

R₁: carbocyclic (saturated and unsaturated), heterocyclic (saturated or unsaturated), alkyls (saturated and unsaturated). Examples include phenyl, benzyl, pyrimidyl, naphthyl, indolyl, pyrrolyl, furyl, thienyl, imidazolyl, cyclohexyl, piperidyl, pyrrolidyl, morpholinyl, cyclohexenyl, butadienyl, and the like.

R₁ can further include substitutions on the carbocyclic or heterocyclic moieties with substituents such as halogen, nitro, amino, hydroxyl, alkoxy, carboxyl, cyano, thio, alkyl, aryl, heteroalkyl, heteroaryl and combinations thereof, for example, 4-nitrophenyl, 3-chlorophenyl, 2,5-dinitrophenyl, 4-methoxyphenyl, 5-methyl-pyrrolyl, 2,5-dichlorocyclohexyl, guanidinyl-cyclohexenyl and the like.

R₂: alkyl, carbocylic, aryl, heterocyclic. Examples include: methyl, ethyl, propyl, isopropyl, phenyl, 4-nitrophenyl, thienyl and the like.

X: may be any number (from 1 to 3) of substituents on the carbocyclic or heterocyclic ring. The substituents can be the same or different. Substituents can include hydrogen, alkyl, heteroalkyl, aryl, heteroaryl, halo, nitro, carboxyl, hydroxyl, cyano, amino, thio, alkylamino, haloaryl and the like.

The substituents may be optionally further substituted with 1-3 substituents from the group consisting of alkyl, aryl, nitro, alkoxy, hydroxyl and halo groups. Examples include: methyl, 2,3-dimethyl, phenyl, p-tolyl, 4-chlorophenyl, 4-nitrophenyl, 2,5-dichlorophenyl, furyl, thienyl and the like.

Specific Examples include:

TABLE 2

| IA | IIB |
|---|---|
| 5-Methyl-1-(2′-pyridyl)-2-(1H) pyridine, | 6-Methyl-1-phenyl-3-(1H) pyridone, |
| 6-Methyl-1-phenyl-2-(1H) pyridone, | 5-Methyl-1-p-tolyl-3-(1H) pyridone, |
| 5-Methyl-3-phenyl-1-(2′-thienyl)-2-(1H) pyridone, | 5-Methyl-1-(2′-naphthyl)-3-(1H) pyridone, |
| 5-Methyl-1-(2′-naphthyl)-2-(1H) pyridone, | 5-Methyl-1-phenyl-3-(1H) pyridone, |
| 5-Methyl-1-p-tolyl-2-(1H) pyridone, | 5-Methyl-1-(5′-quinolyl)-3-(1H) pyridone, |
| 5-Methyl-1-(1′naphthyl)-2-(1H) pyridone, | 5-Ethyl-1-phenyl-3-(1H) pyridone, |
| 5-Ethyl-1-phenyl-2-(1H) pyridone, | 5-Methyl-1-(4′-methoxyphenyl)-3-(1H) pyridone, |
| 5-Methyl-1-(5′-quinolyl)-2-(1H) pyridone, | 4-Methyl-1-phenyl-3-(1H) pyridone, |
| 5-Methyl-1-(4′-quinolyl)-2-(1H) pyridone, | 5-Methyl-1-(3′-pyridyl)-3-(1H) pyridone, |
| 5-Methyl-1-(4′-pyridyl)-2-(1H) pyridone, | 5-Methyl-1-(2′-Thienyl)-3-(1H) pyridone, |
| 3-Methyl-1-phenyl-2-(1H) pyridone, | 5-Methyl-1-(2′-pyridyl)-3-(1H) pyridone, |
| 5-Methyl-1-(4′-methoxyphenyl)-2-(1H)pyridone, | 5-Methyl-1-(2′-quinolyl)-3-(1H) pyridone, |
| 1-Phenyl-2-(1H) pyridone, | 1-Phenyl-3-(1H) pyridine, |
| 1,3-Diphenyl-2-(1H) pyridone, | 1-(2′-Furyl)-5-methyl-3-(1H) pyridone, |
| 1,3-Diphenyl-5-methyl-2-(1H) pyridone, | 1-(4′-Chlorophenyl)-5-methyl-3-(1H) pyridine. |
| 5-Methyl-1-(3′-trifluoromethylphenyl)-2- | |
| (1H)-pyridone, | |
| 3-Ethyl-1-phenyl-2-(1H) pyridone, | |
| 5-Methyl-1-(3′-pyridyl)-2-(1H) pyridone, | |
| 5-Methyl-1-(3-nitrophenyl)-2-(1H) pyridone, | |
| 3-(4′-Chlorophenyl)-5-Methyl-1-phenyl-2- | |
| (1H) pyridone, | |
| 5-Methyl-1-(2′-Thienyl)-2-(1H) pyridone, | |
| 5-Methyl-1-(2′-thiazolyl)-2-(1H) pyridone, | |
| 3,6-Dimethyl-1-phenyl-2-(1H) pyridone, | |
| 1-(4′Chlorophenyl)-5-Methyl-2-(1H) pyridone, | |
| 1-(2′-Imidazolyl)-5-Methyl-2-(1H) pyridone, | |
| 1-(4′-Nitrophenyl)-2-(1H) pyridone, | |
| 1-(2′-Furyl)-5-Methyl-2-(1H) pyridone, | |
| 1-Phenyl-3-(4′-chlorophenyl)-2-(1H) pyridine. | |

U.S. Pat. Nos. 3,974,281; 3,839,346; 4,042,699; 4,052,509; 5,310,562; 5,518,729; 5,716,632; and 6,090,822 describe methods for the synthesis and formulation of pirfenidone and specific pirfenidone analogs in pharmaceutical compositions suitable for use in the methods of the present invention.

In one aspect, the invention provides any of the methods described herein in which the subject method is modified to include co-administering to the patient a therapeutically effective amount of pirfenidone or a pirfenidone analog for the duration of the desired course of interferon receptor agonist treatment.

In another aspect, the invention provides any of the methods described herein in which the subject method is modified to include co-administering to the patient about 400 mg to about 3600 mg of pirfenidone, or a specific pirfenidone analog, orally qd for the duration of the desired course of the interferon receptor agonist treatment.

In another aspect, the invention provides any of the methods described herein in which the subject method is modified to include co-administering to the patient about 25 mg to about 125 mg pirfenidone, or a specific pirfenidone analog, per kg of the patient's body weight orally qd for the duration of the desired course of interferon receptor agonist treatment.

### Liver Targeting Systems

Antiviral agents described herein can be targeted to the liver, using any known targeting means. Those skilled in the art are aware of a wide variety of compounds that have been demonstrated to target compounds to hepatocytes. Such liver targeting compounds include, but are not limited to, asialoglycopeptides; basic polyamino acids conjugated with galactose or lactose residues; galactosylated albumin; asialoglycoprotein-poly-L-lysine) conjugates; lactosaminated albumin; lactosylated albumin-poly-L-lysine conjugates; galactosylated poly-L-lysine; galactose-PEG-poly-L-lysine conjugates; lactose-PEG-poly-L-lysine conjugates; asialofetuin; and lactosylated albumin.

In some embodiments, a liver targeting compound is conjugated directly to the antiviral agent. In other embodiments, a liver targeting compound is conjugated indirectly to the antiviral agent, e.g., via a linker. In still other embodiments, a liver targeting compound is associated with a delivery vehicle, e.g., a liposome or a microsphere, forming a hepatocyte targeted delivery vehicle, and the antiviral agent is delivered using the hepatocyte targeted delivery vehicle.

The terms “targeting to the liver” and “hepatocyte targeted” refer to targeting of an antiviral agent to a hepatocyte, such that at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, of the antiviral agent administered to the subject enters the liver via the hepatic portal and becomes associated with (e.g., is taken up by) a hepatocyte.

### Combination Therapy: Type I, Type II, Type III Interferons

As discussed above, the methods of the invention can be carried out using combinations of a Type I IFN and a Type II IFN; a Type III IFN and a Type III IFN; and a Type II IFN and a Type III IFN.

Type I and Type II Combination Therapy

In some embodiments, the methods of the invention are carried out by administering a combination of a Type I IFN and IFN-γ (a Type II IFN). In many of these embodiments, the Type I IFN is an IFN-α. Effective dosages of an IFN-α are described above. Effective dosages of IFN-γ can range from about 25 μg to about 300 μg, or about 100 μg to about 200 μg.

In some embodiments, the Type I IFN is IFN-ω, and the combination therapy involves administering IFN-α and IFN-γ. Effective dosages of IFN-ω can range from 3 μg to about 320 μg. Effective dosages of IFN-γ can range from about 25 μg to about 300 μg, or about 100 μg to about 200 μg.

In some embodiments, the Type I IFN is IFN-tau, and the combination therapy involves administering IFN-tau and IFN-γ. Effective dosages of IFN-tau range from 3 μg to about 320 μg. Effective dosages of IFN-γ range from about 25 μg to about 300 μg, or about 100 tag to about 200 μg.

In some embodiments, the Type I IFN is IFN-β, and the combination therapy involves administering IFN-β and IFN-γ. Effective dosages of IFN-β can range from 3 μg to about 320 μg, e.g., 30 μg, 40-45 μg, etc. Effective dosages of IFN-γ can range from about 25 μg to about 300 μg, or about 100 μg to about 200 μg.

Type II and Type III Combination Therapy

In some embodiments, the methods provide for administration of IFN-γ in combination therapy with a Type III IFN. For example, IL-28A, IL-28B, or IL29 is administered in combination therapy with IFN-γ. Effective dosages of a Type III IFN can range from 3 μg to about 320 μg. Effective dosages of IFN-γ can range from about 25 μg to about 300 μg, or about 100 μg to about 200 μg.

Type I and Type II Combination Therapy

In some embodiments, the methods provide for administration of a Type I IFN in combination therapy with a Type III IFN. In many of these embodiments, the Type I IFN is an IFN-α. Effective dosages of an IFN-α are described above. Effective dosages of a Type III IFN can range from about 3 μg to about 320 μg.

### Determining Effectiveness of Treatment

Whether a subject method is effective in treating a hepatitis virus infection, particularly an HCV infection, can be determined by measuring viral load, or by measuring a parameter associated with HCV infection, including, but not limited to, liver fibrosis.

Viral load can be measured by measuring the titer or level of virus in serum. These methods include, but are not limited to, a quantitative polymerase chain reaction (PCR) and a branched DNA (bDNA) test. For example, quantitative assays for measuring the viral load (titer) of HCV RNA have been developed. Many such assays are available commercially, including a quantitative reverse transcription PCR (RT-PCR) (Amplicor HCV Monitor™, Roche Molecular Systems, New Jersey); and a branched DNA (deoxyribonucleic acid) signal amplification assay (Quantiplex™: HCV RNA Assay (bDNA), Chiron Corp., Emeryville, Calif.). See, e.g., Gretch et al. (1995) Ann. Intern Med 123:321-329.

As noted above, whether a subject method is effective in treating a hepatitis virus infection, e.g., an HCV infection, can be determined by measuring a parameter associated with hepatitis virus infection, such as liver fibrosis. Liver fibrosis reduction is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

As one non-limiting example, levels of serum alanine aminotransferase (ALT) are measured, using standard assays. In general, an ALT level of less than about 45 international units per milliliter serum is considered normal. In some embodiments, an effective amount of an interferon receptor agonist is an amount effective to reduce ALT levels to less than about 45 IU/ml serum.

### Subjects Suitable for Treatment

Individuals who have been clinically diagnosed as infected with a hepatitis virus (e.g., HAV, HBV, HCV, delta, etc.), particularly HCV, are suitable for treatment with the methods of the instant invention. Individuals who are infected with HCV are identified as having HCV RNA in their blood, and/or having anti-HCV antibody in their serum. Such individuals include naïve individuals (e.g., individuals not previously treated for HCV, particularly those who have not previously received IFN-α-based or ribavirin-based therapy) and individuals who have failed prior treatment for HCV (“treatment failure” patients). Treatment failure patients include non-responders (e.g., individuals in whom the HCV titer was not significantly or sufficiently reduced by a previous treatment for HCV, particularly a previous IFN-α monotherapy using a single form of IFN-α); and relapsers (e.g., individuals who were previously treated for HCV (particularly a previous IFN-α monotherapy using a single form of IFN-α), whose HCV titer decreased significantly, and subsequently increased). In particular embodiments of interest, individuals have an HCV titer of at least about 10⁵, at least about 5×10⁵, or at least about 10⁶, genome copies of HCV per milliliter of serum. The patient may be infected with any HCV genotype (genotype 1, including 1a and 1b, 2, 3, 4, 5, 6, etc. and subtypes (e.g., 2a, 2b, 3a, etc.)), particularly a difficult to treat genotype such as HCV genotype 1 and particular HCV subtypes and quasispecies.

In certain embodiments, the specific regimen of drug therapy used in the treatment of the HCV patient is selected according to certain disease parameters or clinical characteristics exhibited or presented by the patient, such as the initial viral load, genotype of the HCV infection, liver histology, stage of liver fibrosis, and/or antiviral therapeutic history of the patient. The invention contemplates any of the above-described methods for treatment of HCV infection in which the subject method is modified to include performing before the sustained dosage phase, or before the initial dosage phase, or before the initial administration of interferon receptor agonist to the patient, the step or steps of determining patient disease parameter(s) and/or clinical characteristic(s) and using such determination(s) to select the duration of interferon receptor agonist therapy.

In one embodiment, the invention provides any of the above-described methods in which the subject method is modified to include performing before the sustained dosage phase, or before the initial administration of interferon receptor agonist to the patient, or before the initial dosage phase, the steps of (i) identifying the patient as an antiviral treatment naïve patient having a genotype 1 HCV infection and an initial viral load greater than 2 million HCV RNA genome copies/ml of serum, and (ii) selecting a duration of interferon receptor agonist therapy of about 48 weeks.

In another embodiment, the invention provides any of the above-described methods in which the subject method is modified to include performing before the sustained dosage phase, or before the initial administration of interferon receptor agonist to the patient, or before the initial dosage phase, the steps of (i) identifying the patient as an antiviral treatment naïve patient having a genotype 1 HCV infection and an initial viral load less than or equal to 2 million HCV RNA genome copies/ml of serum, and (ii) selecting a duration of interferon receptor agonist therapy of about 24 weeks to about 48 weeks.

In another embodiment, the invention provides any of the above-described methods in which the subject method is modified to include performing before the sustained dosage phase, or before the initial administration of interferon receptor agonist to the patient, or before the initial dosage phase, the steps of (i) identifying the patient as an antiviral treatment naïve patient having a genotype 2 or 3 HCV infection, and (ii) selecting a duration of interferon receptor agonist therapy of about 6 weeks to about 24 weeks.

In another embodiment, the invention provides any of the above-described methods in which the subject method is modified to include performing before the sustained dosage phase, or before the initial administration of interferon receptor agonist to the patient, or before the initial dosage phase, the steps of (i) identifying the patient as an antiviral treatment naïve patient having a genotype 4 HCV infection, and (ii) selecting a duration of interferon receptor agonist therapy of about 48 weeks.

In another embodiment, the invention provides any of the above-described methods in which the subject method is modified to include performing before the sustained dosage phase, or before the initial administration of interferon receptor agonist to the patient, or before the initial dosage phase, the steps of (i) identifying the patient as an antiviral treatment failure patient, and (ii) selecting a duration of interferon receptor agonist therapy of about 24 weeks to about 60 weeks.

In another embodiment, the invention provides any of the above-described methods in which the subject method is modified to include performing before the sustained dosage phase, or before the initial administration of interferon receptor agonist to the patient, or before the initial dosage phase, the steps of (i) identifying the patient as having failed an earlier course of interferon receptor agonist therapy, and (ii) selecting a duration of interferon receptor agonist therapy of about 24 weeks to about 60 weeks.

In another embodiment, the invention provides any of the above-described methods in which the subject method is modified to include performing before the sustained dosage phase, or before the initial administration of interferon receptor agonist to the patient, or before the initial dosage phase, the steps of (i) identifying the patient as having failed an earlier course of IFN-α 2a or 2b therapy, and (ii) selecting a duration of interferon receptor agonist therapy of about 24 weeks to about 60 weeks.

In another embodiment, the invention provides any of the above-described methods in which the subject method is modified to include performing before the sustained dosage phase, or before the initial administration of interferon receptor agonist to the patient, or before the initial dosage phase, the steps of (i) identifying the patient as having failed an earlier course of PEGASYS® peginterferon alfa-2a or PEG-INTRON® peginterferon alfa-2b therapy, and (ii) selecting a duration of interferon receptor agonist therapy of about 24 weeks to about 60 weeks.

In another embodiment, the invention provides any of the above-described methods in which the subject method is modified to include performing before the sustained dosage phase, or before the initial administration of interferon receptor agonist to the patient, or before the initial dosage phase, the steps of (i) identifying the patient as having failed an earlier course of consensus interferon therapy, and (ii) selecting a duration of interferon receptor agonist therapy of about 24 weeks to about 60 weeks.

In another embodiment, the invention provides any of the above-described methods in which the subject method is modified to include performing before the sustained dosage phase, or before the initial administration of interferon receptor agonist to the patient, or before the initial dosage phase, the steps of (i) identifying the patient as having relapsed after responding to an earlier course of IFN-α therapy and as having a genotype 2 or 3 HCV infection, and (ii) selecting a duration of interferon receptor agonist therapy of about 24 weeks to about 48 weeks.

In another embodiment, the invention provides any of the above-described methods in which the subject method is modified to include performing before the sustained dosage phase, or before the initial administration of interferon receptor agonist to the patient, or before the initial dosage phase, the steps of (i) identifying the patient as having relapsed after responding to an earlier course of IFN-α therapy and as having a genotype 1 or 4 HCV infection, and (ii) selecting a duration of interferon receptor agonist therapy of about 48 weeks.

In another embodiment, the invention provides any of the above-described methods in which the subject method is modified to include performing before the sustained dosage phase, or before the initial administration of interferon receptor agonist to the patient, or before the initial dosage phase, the steps of (i) identifying the patient as having not responded to an earlier course of IFN-α therapy, and (ii) selecting a duration of interferon receptor agonist therapy of about 48 weeks to about 60 weeks.

In connection with each of the methods tailored to the disease parameter(s) and/or other characteristics of the patient described above, the invention also contemplates co-administering to the patient a therapeutically effective amount of ribavirin for the duration of the desired course of interferon receptor agonist therapy. In one embodiment, the subject method includes co-administering to the patient about 800 mg to about 1200 mg ribavirin orally per day, the daily dosage optionally being divided into two doses per day, for the duration of the desired course of interferon receptor agonist therapy. In another embodiment, the subject method includes co-administering to the patient for the duration of the desired course of interferon receptor agonist therapy (a) 1000 mg ribavirin orally per day if the patient has a body weight less than 75 kg or (b) 1200 mg ribavirin orally per day if the patient has a body weight greater than or equal to 75 kg, where the daily dosage is optionally divided into two doses per day.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Example 1

### Study Evaluating the Safety and Efficacy of Infergen Administration by Continuous Delivery Using a Subcutaneous Pump in Combination with Oral Ribavirin in Patients Infected with Hepatitis C Virus who are Treatment Naïve or Peginterferon-Alfa Plus Ribavirin Non-Responders

### Study Drugs/Device

Infergen (interferon alfacon-1 [also known as consensus interferon or CIFN]); ribavirin 200 mg capsules; and Medtronic, Inc. MiniMed Pump—Model 508.

### Objectives

Primary Objective: Evaluate the safety and tolerability of Infergen administered by continuous infusion at 2 doses (12 μg/day or 18 μg/day) using a subcutaneous pump in combination with daily oral ribavirin in HCV-infected patients who have failed pegylated interferon therapy, or who are therapy naïve.

Secondary Objectives Assess the early viral response at (Weeks 1, 4, 12, and 24); assess the sustained viral response (Week 72); and assess the pharmacokinetic profile of Infergen delivered by continuous infusion.

### Study Design

Open-label, single-arm, dose escalation study evaluating Infergen inked continuously using the MiniMed pump, at doses of 12 μg/day and 18 μg/day. Daily oral ribavirin based on weight (subjects ≦75 kg receive 1000 mg daily; subjects >75 kg receive 1200 mg daily).

### Methods

Patients will be dosed in three groups. The first group of 6 patients will be peginterferon-alfa/ribavirin nonresponders who will begin continuous infusion of Infergen at 12 μg/day for 4 weeks. If more than 2 patients require dose reduction or discontinuation by week 4 due to tolerability, drug-related SAE, or drug-related grade 3 or higher abnormal laboratory value, the remaining patients will continue with the 12 μg/day regimen rather than escalating to 18 μg/day. All subjects will be evaluated at screening, days 1, 3, 7, 10, 14, 21, 28 and then at weeks 6 and 12 for HCV RNA.

If there is no dose limiting toxicity in the first group of nonresponders these patients will be permitted to increase the Infergen dose to 18 μg/day at week 6, if the following criteria are met: HCV RNA positive with <2 log reduction in viral load at week 4. If the virus persists after 24 weeks of treatment, the patient will be discontinued from study treatment and will return for a follow-up visit at week 28. All other subjects will continue on the assigned treatment regimen until week 48 and will be evaluated at the study site at weeks 16, 20, 24, 32, 40, 48, 60 and 72.

At approximately week 6 of the study, the second group (6 patients) of nonresponders and the treatment naïve patient group (10 patients) will begin enrolling concurrently if there is not a dose limiting toxicity (DLT) in the first group of patients as defined by protocol. The second group of nonresponders will start Infergen treatment at 18 μg/day, while treatment naïve patients will receive 12 μg/day.

Treatment naïve patients who do not have a >2 log reduction in viral load and are HCV RNA positive at week 12 will discontinue treatment and return for a follow-up evaluation at week 16. All other subjects will continue on the assigned treatment regimen until week 48 and will be evaluated at the study site at weeks 16, 20, 24, 32, 40, 48, 60 and 72.

Subjects will be contacted by phone at weeks 28, 36, 44, and 52 to evaluate adverse events and pregnancy status in females of child bearing potential.

Blood will be collected at the following time points: screening visit within 4 weeks of entry, days 1, 3, 7, 10, 14, 21, and 28; weeks 6, 9, 12, 16, 20, 24, 32, 40, 48, 60, and 72 for laboratory and safety evaluation and pharmacokinetic parameters.

### Sample Size

Total of 10 therapy naïve patients and 12 peginterferon-alfa/ribavirin nonresponders.

### Subject Characteristics

HCV infected patients 18-50 years old who have documented chronic HCV infection and were previously treated with peginterferon-alfa and ribavirin for a minimum of 12 weeks and terminated treatment because of viral non-response, or are therapy naïve.

### Summary of Eligibility Criteria

Men and women 18-50 years old with who have documented chronic HCV infection and were previously treated with peginterferon-alfa and ribavirin for a minimum of 12 weeks who terminated treatment because of viral non-response, as well as therapy naïve patients. Viral non-response is defined by the presence of HCV RNA in blood at the end of a minimum of 24 weeks of therapy, or lack of >2 log reduction in HCV RNA after 12 weeks of therapy. In addition, the patients should have a serum ALT above the upper limit of normal, clinically compensated liver disease, the capability to understand and execute a signed informed consent, and demonstrate the ability to operate an external infusion pump.

Patients who have received non pegylated interferon therapy in combination with ribavirin and were nonresponders are to be excluded. Patients who were treated with peginterferon-alfa who relapsed (i.e. initially cleared virus but were found later to be positive for HCV RNA) during the follow-up period are to be excluded.

Patients with other forms of liver disease, clinically significant anemia, hepatocellular carcinoma, hepatitis A or hepatitis B infection, significant cardiac disease, renal disease, seizure disorder, autoimmune disease, retinopathy, presence of severe mental depression or other psychiatric disease, abnormal thyroid function which cannot be maintained by medication, HIV positive, total bilirubin >2.0 mg/dL (unless due to Gilbert's syndrome), platelet count ≦75×10⁹/L, absolute neutrophil count ≦1.5×10⁹/L, hemoglobin <12 g/dL in women or <13 g/dL in men, or serum creatinine 1.5 times ULN, are to be excluded.

Patients with cirrhosis may not be enrolled into the PK portion of the study.

Female patients who are pregnant or lactating, and male partners of women who are pregnant are excluded.

### Drug Formulation and Route of Administration

Interferon-alfacon (Infergen®), InterMune Inc.: 12 μg/day or 18 μg/day administered by continuous infusion via subcutaneous pump (prescription to be written). Infergen is available as a sterile, preservative-free liquid in single entry vials containing 9 μg or 15 μg at a fill volume of 0.3 mL and 0.5 mL, respectively.

Ribavirin (Rebetol®), Schering-Plough: 1000 mg/day po or 1200 mg/day po depending on weight (prescription to be written). Each capsule contains 200 mg of ribavirin produced by Schering Plough. The capsules are taken orally in two divided doses.

MiniMed Pump—Model 407C, manufactured by Medtronic, Inc.

### Measures of Safety and Efficacy

Safety: Local and systemic tolerability, physical examination including vital signs, adverse events, laboratory safety tests, and assessment of study withdrawals due to adverse events.

Efficacy: Sustained viral response defined as the absence of detectable HCV RNA in plasma samples at 24 weeks or longer after the completion of study therapy, as assessed by HCV RNA PCR detection methods performed at a central laboratory. Patients who withdraw at Week 12 with a <2 log reduction in HCV RNA will be considered failures to attain sustained viral response.

Pharmacokinetics (PK): It is expected that constant infusion of Infergen will result in zero-order kinetics. Blood levels will be assessed periodically to confirm steady state drug concentration and elimination kinetics when therapy is discontinued.

### Statistical Analyses

Statistical analyses of data will be performed using observational study reporting rates of response and rates of adverse events. There is no comparison group.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of treating a hepatitis C virus (HCV) infection in an individual, the method comprising administering a therapeutically effective amount of an IFN-α to the individual in an initial dosage phase followed by a sustained dosage phase consisting of at least one sustained dosage interval, wherein during the initial dosage phase an initial serum concentration of the IFN-α is achieved within a first period of time of about 12 hours to about 48 hours, wherein during the first sustained dosage interval a first sustained serum concentration of the IFN-α of at least about 80% and up to about 200% of the initial serum concentration is achieved and maintained at a substantially steady state for a period of time of at least about 5 days, and for any following sustained dosage interval a following sustained serum concentration of the IFN-α of at least about 20% of the first sustained serum concentration and at least about 50% and up to about 200% of the sustained serum concentration in the preceding sustained dosage interval is achieved and maintained at a substantially steady state for a period of at least about 5 days, and the duration of the IFN-α therapy is at least about 6 weeks.

2. The method of claim 1, wherein the IFN-α is administered to the individual during at least the sustained dosage phase in a substantially continuous manner.

3. The method of claim 2, wherein the IFN-α is administered to the individual during at least the sustained dosage phase in a substantially continuous manner by an implantable infusion pump.

4. The method of claim 3, wherein the pump administers to the individual a single bolus dose of the IFN-α to achieve the initial serum concentration during the initial dosage phase and then administers to the individual a pre -selected amount of the IFN-α per day by continuous infusion to achieve and maintain the sustained serum concentration for each sustained dosage interval.

5. The method of claim 4, wherein the infusion pump is implanted for subcutaneous delivery and the bolus dose is at least about 3 million International Units (IU) of the IFN-α.

6. The method of claim 5, wherein the sustained dosage phase consists of a single: sustained dosage interval and the pre-selected amount of the IFN-α in the sustained dosage interval is at least about 3 million IU of the IFN-α per day.

7. The method of claim 1, wherein a single bolus dose of the IFN-α is administered to the individual by subcutaneous injection to achieve the initial serum concentration during the initial dosage phase.

8. The method of claim 7, wherein the IFN-α is administered to the individual in a substantially continuous manner by an implantable infusion pump that delivers a preselected amount of the IFN-α per day to achieve and maintain the sustained serum concentration for each sustained dosage interval.

9. The method of claim 1, wherein the IFN-α is administered to the individual during the initial and sustained dosage phases in a substantially continuous manner.

10. The method of claim 9, wherein IFN-α is administered to the individual during the initial and sustained dosage phases in a substantially continuous manner by an implantable infusion pump.

11. The method of claim 10, wherein the implantable infusion pump is controlled to deliver a pre-selected amount of the IFN-α per day to achieve the initial serum concentration during the initial dosage phase and to achieve and maintain the sustained serum concentration for each sustained dosage interval.

12. The method of claim 8, wherein the pre-selected amount of the IFN-α is at least about 9 million International Units (IU) of the IFN-α per day and is administered to the individual by subcutaneous infusion.

13. The method of claim 1, wherein each sustained serum concentration is at least about 95% of the initial serum concentration.

14. The method of claim 1, wherein the sustained dosage phase consists of a single sustained dosage interval and the initial- and sustained serum concentrations are substantially the same.

15. A method of treating hepatitis C virus (HCV) infection in an individual, the method comprising administering a therapeutically effective amount of an IFN-α to the individual for a treatment period of at least about 6 weeks, wherein a sustained serum concentration of the IFN-α is achieved and maintained at a substantially steady state during the treatment period, and wherein the therapeutically effective amount of the IFN-α administered during the treatment period is at least about 3 million International Units (IU) per day.

16. The method of claim 1, wherein the sustained serum concentration of the IFN-α in the last sustained dosage interval of the sustained dosage phase or in the treatment period is at least about 90% of the maximum tolerated dose (MTD) of the individual.

17. The method of claim 1, wherein the sustained serum concentration of the IFN-α in the last sustained dosage interval of the sustained dosage phase or in the treatment period is at least about 95% of the maximum tolerated dose (MTD) of the individual.

18. The method of claim 1, wherein for the treatment period or for each sustained dosage interval the area under the curve defined by the serum concentration of the IFN-α as a function of time for any 8 hour period in the treatment period or sustained dosage interval (AUC₈ₕᵣ) is no more than about 20% above or about 20% below an average serum concentration (AUC_{8hr Average}), wherein the AUC AUC_{8hr Average} is equal to the quotient of the area under the curve defined by serum concentration of the IFN-α as a function of time for the entirety of the treatment period or sustained dosage interval (AUCₜₒₜₐₗ) divided the number of 8 hour segments in the treatment period or sustained dosage interval (t_{total1/3 days}).

19. The method of claim 1, wherein the IFN-α is a consensus interferon.

20. The method of claim 19, wherein the consensus interferon is INFERGEN® interferon alfacon 1.

21. The method of claim 1, wherein the IFN-α is IFN-α2a or IFN-α2b.

22. The method of claim 1, further comprising administering to the individual a therapeutically effective amount of ribavirin for the duration of the IFN-α therapy.

23. The method of claim 1, further comprising administering to the individual about 800 mg to about 1200 mg ribavirin orally per day for the duration of the IFN-α therapy.

24. The method of claim 1, further comprising administering to the individual (a) 1000 mg ribavirin orally per day if the individual has a body weight less than 75 kg or (b) 1200 mg ribavirin orally per day if the individual has a body weight greater than or equal to 75 kg, wherein the daily dosage of ribavirin is administered to the individual in 2 divided doses per day for the duration of the IFN-α therapy.

25. A method of treating a hepatitis C virus (HCV) infection in an individual, comprising administering a therapeutically effective amount of an IFN-α to the individual in an initial dosage phase followed by a sustained dosage phase consisting of at least one sustained dosage interval, wherein the initial dosage phase extends for a period of time of about 12 hours to about 48 hours and an initial pre-selected amount of the IFN-α is administered to the individual by a selected route of administration during the initial dosage phase, wherein during the first sustained dosage interval a first sustained pre-selected amount of the IFN-α is administered to the individual each day by the selected route of administration in a substantially continuous manner for a period of time of at least about 5 days, wherein the first sustained pre-selected amount of the IFN-α is at least about 80% and up to about 200% of the initial pre-selected amount of the IFN-α, and for any following sustained dosage interval a following sustained pre-selected amount of the IFN-α is administered to the individual each day by the selected route of administration in a substantially continuous manner for a period of time of at least about 5 days, wherein the following sustained pre-selected amount of the IFN-α is at least about 20% of the first sustained pre-selected amount and at least about 50% and up to about 200% of the sustained preselected amount in the preceding sustained dosage interval, and wherein the duration of the IFN-α therapy is at least about 6 weeks.

26. A method of treating hepatitis C virus (HCV) infection in an individual, the method comprising administering to the individual a therapeutically effective amount of an IFN-α for a treatment period of at least about 6 weeks, wherein a sustained pre-selected amount of the IFN-α is administered to the individual each day by substantially continuous delivery during the treatment period, and wherein the therapeutically effective amount of the IFN-α administered during the treatment period is at least about 3 million International Units (IU) per day.

27. A method for treating hepatitis C virus (HCV) infection in an individual, the method comprising administering to the individual a therapeutically effective amount of an IFN-α for a treatment period of at least about 6 weeks, wherein each day of the treatment period the individual receives an amount of the IFN-α that is no more than about 20% above or about 20% below an average daily dosage of the IFN-α (ADD_{IFN-α}), and wherein the ADD_{IFN-α} is equal to the aggregate amount of the IFN-α administered to the individual in the treatment period divided by the number of days in the treatment period, and wherein the ADD_{IFN-α} administered during the treatment period is at least about 3 million International Units (IU) per day.

28. The method of claim 25, wherein in the sustained dosage phase the IFN-α is administered to the individual in a substantially continuous manner by an implantable infusion pump.

29. The method of claim 28, wherein in the initial dosage phase the pump is implanted and used to administer the initial pre-selected amount of the IFN-α as a bolus at the beginning of the initial dosage phase.

30. The method of claim 25, wherein the initial pre-selected amount of the IFN-α is administered by bolus injection at the beginning of the initial dosage phase.

31. The method of claim 25, wherein in the treatment period or in the initial and sustained dosage phases the IFN-α is administered to the individual in a substantially continuous manner by an implantable infusion pump.

32. The method of claim 25, wherein the IFN-α is administered to the individual subcutaneously during the treatment period or the initial and sustained dosage phases.

33. The method of claim 25, wherein the IFN-α is a consensus interferon.

34. The method of claim 33, wherein the consensus interferon is Infergen® interferon alfacon-1.

35. The method of claim 25, wherein the IFN-α is IFN-α2a or IFN-α2b.

36. The method of claim 25, wherein the ADD_{IFN-α} or the sustained pre-selected amount of the IFN-α in the treatment period or in the last sustained dosage interval of the sustained dosage phase is at least about 3 million International Units (IU) administered subcutaneously.

37. The method of claim 33, wherein the ADD_{IFN-α} or the sustained pre-selected amount of the consensus interferon in the treatment period or in the last sustained dosage interval of the sustained dosage phase is selected from the group consisting of at least about 9 μg, 15 μg, 18 μg, 21 μg, 27 μg, and 30 μg of the consensus interferon administered subcutaneously.

38. The method of claim 25, further comprising to the individual a therapeutically effective amount of ribavirin for the duration of the IFN-α therapy.

39. The method of claim 25, further comprising administering to the individual about 800 mg to about 1200 mg ribavirin orally per day for the duration of the IFN-α therapy.

40. The method of claim 25, further comprising administering to the individual (a) 1000 mg ribavirin orally per day if the individual has a body weight less than 75 kg or (b) 1200 mg ribavirin orally per day if the individual has a body weight greater than or equal to 75 kg, wherein the daily dosage of ribavirin is administered to the individual in 2 divided doses per day for the duration of the IFN-α therapy.

41. A method for treating an individual having a hepatitis C virus (HCV) infection, the method comprising administering to the individual an effective amount of an IFN-α for a treatment period of at least about 6 weeks, wherein the area under the curve of serum concentration of the IFN-α over time for any 8 hour interval in the treatment period is no more than about 20% above or about 20% below the average area under the curve of serum concentration of the IFN-α over time for an 8 hour interval in the treatment period (AUC_{8hr average}), and wherein the AUC_{8hr average} is equal to the area under the curve of serum concentration of the IFN-α over the entirety of the treatment period (AUCₜₒₜₐₗ) divided by the number of 8 hour intervals in the treatment period.

42. The method of claim 1, wherein the duration of the IFN-α therapy is at least about 24 weeks.

43. The method of claim 1, wherein the duration of the IFN-α therapy is at least about 48 weeks.

44. The method of claim 1, wherein the individual is an antiviral treatment naïve patient having a genotype 1 HCV infection and an initial viral load of greater than 2 million HCV RNA genome copies/ml of serum, and wherein the duration of the IFN-α therapy is about 48 weeks.

45. The method of claim 1, wherein the individual is an antiviral treatment naïve patient having a genotype 1 HCV infection and an initial viral load of less than or equal to 2 million HCV RNA genome copies/ml of serum, and wherein the duration of the IFN-α therapy is about 24 weeks to about 48 weeks.

46. The method of claim 1, wherein the individual is an antiviral treatment naïve patient having a genotype 4 HCV infection, and wherein the duration of the IFN-α therapy is about 48 weeks.

47. The method of claim 1, wherein the individual is an antiviral treatment naïve patient having a genotype 2 or 3 HCV infection, and wherein the duration of the IFN-α therapy is about 6 weeks to about 24 weeks.

48. The method of claim 1, wherein the individual is an antiviral treatment failure patient and the duration of the IFN-α therapy is about 24 weeks to about 60 weeks.

49. The method of claim 48, wherein the antiviral treatment failure patient failed at least one earlier course of IFN-α monotherapy for HCV infection.

50. The method of claim 48, wherein the antiviral treatment failure patient failed at least one earlier course of IFN-α and ribavirin combination therapy for HCV infection.

51. The method of claim 49, wherein the earlier course of IFN-α therapy was either IFN-α2a or IFN-α2b therapy.

52. The method of claim 49, wherein the earlier course of IFN-α therapy was either peginterferon alfa-2a or peginterferon alfa-2b therapy.

53. The method of claim 51, wherein the IFN-α is a consensus interferon.

54. The method of claim 49, wherein the individual relapsed after responding to the earlier course of IFN-α therapy and has a genotype 2 or 3 HCV infection, and wherein the duration of the IFN-α therapy is about 24 weeks to about 48 weeks.

55. The method of claim 49, wherein the individual relapsed after responding to the earlier course of IFN-α therapy and has a genotype 1 or 4 HCV infection, and wherein the duration of the IFN-α therapy is about 48 weeks.

56. The method of claim 49, wherein the individual did not respond to the earlier course of IFN-α therapy, and wherein the duration of the IFN-α therapy is about 48 weeks to about 60 weeks.

57. The method of claim 1, wherein before the initial administration of IFN-α to the individual (a) the individual is identified as an antiviral treatment naïve patient having a genotype 1 HCV infection and an initial viral load of greater than 2 million HCV RNA genome copies/ml of serum and the duration of the IFN-α therapy is set at about 48 weeks (b) the individual is identified as an antiviral treatment naïve patient having a genotype 1 HCV infection and an initial viral load of less than or equal to 2 million HCV RNA genome copies/ml of serum and the duration of the IFN-α therapy is set at about 24 weeks to about 48 weeks (c) the individual is identified as an antiviral treatment naïve patient having a genotype 2 or 3 HCV infection and the duration of the IFN-α therapy is set at about 6 weeks to about 24 weeks (d) the individual is identified as an antiviral treatment naïve patient having a genotype 4 HCV infection and the duration of the IFN-α therapy is set at about 48 weeks (e) the individual is identified as having relapsed after responding to an earlier course of IFN-α therapy and as having a genotype 1 or 4 HCV infection and the duration of the IFN-α therapy is set at about 48 weeks (f) the individual is identified as having relapsed after responding to an earlier course of IFN-α therapy and as having a genotype 2 infection and the duration of the IFN-α therapy is set at about 24 weeks to about 48 weeks or (g) the individual is identified as having failed to respond to an earlier course of IFN-α therapy and the duration of the IFN-α therapy is set at about 48 weeks to about 60 weeks.

58. A method of treating hepatitis C infection (HCV) in an individual, comprising administering to the individual a therapeutically effective amount of an IFN-α for a treatment period of at least about 6 weeks, wherein a pre-selected amount of the IFN-α is administered to the individual each day, wherein at least about 50% of the pre-selected amount of the IFN-α is delivered as a bolus at the beginning or within the first hour of a sleeping period of about 8 hours to about 12 hours and the undelivered remainder of the pre-selected amount is delivered continuously during the balance of time remaining after the sleeping period in each 24 hour interval in the treatment period.

59. The method of claim 58, wherein an implantable infusion pump is used to perform the bolus and continuous delivery of the IFN-α to the individual, and wherein the pump is controlled to deliver the bolus at the beginning or within the first hour of a sleeping period of about 10 hours and the remainder during the balance of time remaining after the sleeping period in each 24 hour interval in the treatment period.

60. A method of treating hepatitis C virus (HCV) infection in an individual, comprising administering to the individual a therapeutically effective amount of an IFN-α for a treatment period of at least about 6 weeks, wherein a pre-selected amount of the IFN-α per day is administered to the individual in a bolus pulse delivery cycle that is repeated each day for the duration of the treatment period, wherein the bolus pulse delivery cycle consists of all least three bolus dose administrations of the IFN-α separated by evenly spaced intervals of time in a 24 hour cycle, and wherein the aggregate of the bolus dose administrations in each 24 hour cycle equals the pre-selected amount of the IFN-α per day.

61. The method of claim 58, wherein the duration of the IFN-α therapy and the characteristics of the individual are as provided in claim 44.

62. The method of claim 58, wherein the duration of the IFN-α therapy is set according to clauses (a)-(g) in claim 57.

63. The method of claim 58, further comprising administering to the individual a therapeutically effective amount of ribavirin for the duration of the IFN-α therapy.

64. The method of claim 58, further comprising administering to the individual about 800 mg to about 1200 mg ribavirin orally per day for the duration of the IFN-α therapy.

65. The method of claim 58, further comprising administering to the individual (a) 1000 mg ribavirin orally per day if the individual has a body weight less than 75 kg or (b) 1200 mg ribavirin orally per day if the individual has a body weight greater than or equal to 75 kg, wherein the daily dosage of ribavirin is administered to the individual in 2 divided doses per day for the duration of the IFN-α therapy.

66. The method of claim 1, wherein the individual is a human.

67. The method of claim 1, wherein the IFN-α is an unPEGylated IFN-α.

68. An apparatus for the administration of an IFN-α to an individual having a hepatitis C virus (HCV) infection, comprising:
(a) a device for the delivery of the IFN-α to the individual by a selected route of administration; and
(b) a control unit operated by a series of commands comprising a set of instructions that causes the device to administer to the individual the therapeutically effective amount of the IFN-α by the selected route of administration according to the method of claim 1, wherein the control unit executes the set of instructions in the series of commands after the apparatus is installed on the individual, armed for operation, and activated to administer the IFN-α to the individual.


SEQUENCE LISTING (1 sequence)

SEQ ID NO: 1 - protein, 167 aa, Artificial Sequence
  consensus sequence
MCDLPQTHSLGNRRALILLAQMRRISPFSCLKDRHDFGFPQEEFDGNQFQKAQAISVLHEMIQQTFNLFSTKDSSAAWDESLLEKFYTELYQQLNDLEACVIQEVGVEETPLMNVDSILAVKKYFQRITLYLTEKKYSPCAWEVVRAEIMRSFSLSTNLQERLRRKE